(19) United States
(12) Patent Application Publication
(10) Pub. No.: US 2024/0226295 A9
(43) Pub. Date: Jul. 11, 2024

(54) CELL THERAPY COMPOSITIONS AND METHODS FOR MODULATING TGF-B SIGNALING

(71) Applicant: TAKEDA PHARMACEUTICAL COMPANY LIMITED, Osaka (JP)

(72) Inventors: Chantal KUHN, Acton, MA (US); Gary SHAPIRO, Arlington, MA (US)

(21) Appl. No.: 18/546,384

(22) PCT Filed: Feb. 15, 2022
(86) PCT No.: PCT/IB2022/000063

§ 371 Date: Aug. 14, 2023

Related U.S. Application Data
(60) Provisional application No. 63/149,628, filed on Feb. 15, 2021.
(60) Provisional application No. 63/306,836, filed on Feb. 4, 2022.

(51) Int. Cl. A61K 39/00; A61K 35/17; A61P 35/00; A61P 37/02; C12N 5/0783; C12N 15/63

(52) U.S. Cl. CPC A61K 39/4631; A61K 35/17; A61P 35/00; A61P 37/02; C12N 5/0636; C12N 15/63; A61K 2239/11; A61K 2239/21; A61K 2239/22; C12N 2510/00

(57) ABSTRACT

Methods of using polypeptides to modulate transforming growth factor-β (TGFβ) signaling (e.g., TGFβ receptors, antibodies or antigen-binding fragments thereof that specifically bind TGFβ or a TGFβ receptor) are provided. Compositions comprising the antibodies or fragments thereof and methods of using the same for treatment of diseases involving TGFβ activity are provided. Nucleic acids, recombinant expression vectors, host cells, antigen binding fragments, and pharmaceutical compositions comprising these antigen binding agents and fragments thereof are also disclosed. The invention also provides therapeutic methods for utilizing the TGFβ signaling modulators are provided herein.

### CROSS-REFERENCE TO RELATED APPLICATIONS

This application claims priority to U.S. Provisional Application No. 63/149,628, filed Feb. 15, 2021, and U.S. Provisional Application No. 63/306,836, filed Feb. 4, 2022, the disclosure of each of which is hereby incorporated by reference in its entirety.

### BACKGROUND

Immunotherapy using engineered cells that target cancer specific antigens has shown efficacy in treatment for some cancers. However, malignant cells adapt to generate an immunosuppressive microenvironment to protect themselves from immune recognition and elimination. High levels of TGFβ in the tumor microenvironment can contribute to the maintenance and progression of some types of cancer cells. The tumor microenvironment poses significant challenges to methods of treatment involving stimulation of an immune response, such as in case of targeted cell therapies. Accordingly, novel therapeutic strategies for treating cancers are desirable.

### SUMMARY

The present invention provides, among other things, a novel system for modulating TGFβ signaling for use in treating cancers (e.g., solid tumors). The present invention is based in part on the discovery that modulating transforming growth factor-beta (TGF-β) signaling can enhance adoptive cell therapy methods such as targeted engineered chimeric antigen receptor (CAR) therapies. Modulating TGF-β signaling, such as that effected by the system of antibodies (e.g., anti-TGFβ or anti-TGFβR), antigen-binding fragments or recombinant extracellular domain of TGF-βR2, described herein, alleviates the immunosuppressive microenvironment in tumors and potentiates the efficacy of immunotherapy.

T-cell-based immunotherapy has become a new frontier in synthetic biology; multiple promoters and gene products are envisioned to steer these highly potent cells to the tumor microenvironment, where T cells can both evade negative regulatory signals and mediate effective tumor killing. The elimination of unwanted T cells through the drug-induced dimerization of inducible caspase 9 constructs with AP1903 demonstrates one way in which a powerful switch that can control T-cell populations can be initiated pharmacologically (Di Stasi A et al. N Engl J Med. 2011; 365(18):1673-83). Thus, while it appears that CARs can trigger T-cell activation in a manner similar to an endogenous T-cell receptor, a major impediment to the clinical application of this technology to date has been limited in vivo expansion of CAR+ T cells, rapid disappearance of the cells after infusion, and disappointing clinical activity. Accordingly, there is an urgent need in the art for discovering novel compositions and methods for treatment of cancer using an approach that can exhibit specific and efficacious anti-tumor effect without unwanted effects (e.g., high toxicity, insufficient efficacy).

The present invention addresses these needs by providing an immune modulating system comprising a CAR and a TGFβ signaling pathway modulator expressed in an immune cell (e.g., a T cell). Compositions and therapeutic methods comprising the immune modulating system can be used to treat cancers and other diseases and/or conditions. In particular, the present invention provides engineered immune cells expressing armored CARs that may be used for the treatment of diseases, disorders or conditions associated with dysregulated expression of TGFβ (e.g., cancers, solid tumors). Armored CAR T cells co-expressing a TGFβ modulator exhibit high surface expression of the CAR on transduced T cells, and enhanced cytolysis of cancer cells. Thus, the present invention provides methods and compositions for enhancing the immune response toward cancers and pathogens using an immune modulating system (e.g., engineered CAR T cells) comprising a polypeptide that modulates TGF-b signaling.

The present invention provides, in part, improved CAR polypeptides comprising a TGFβ signaling pathway modulator, nucleic acid molecules encoding for such polypeptides, cells (e.g. T cells) genetically modified to express the improved CARs and methods of using the modified cells in adoptive cell therapy for treatment of cancer (e.g., solid tumor cancers).

In some embodiments, the present invention provides CAR-T cells that have been modified to express a TGFβ signaling pathway modulator (also referred to herein as “TGFβ armored CAR-T cells”), such that the cells, when adminstered to a subject in need thereof, are capable of eliciting an immune response in the subject which, relative to a CAR-T cell that does not express a TGFβ signaling pathway modulator (also referred to herein as an “unarmored CAR-T cell”).

In some aspects, the present invention provides immunoresponsive cells (e.g., T cells) bearing antigen receptors, which can be chimeric antigen receptors (CARs), that include polypeptides that modulate TGF-b signaling. These engineered immunoresponsive cells (e.g., CAR-T cells) are antigen-directed and resist immunosuppression and/or have enhanced immune-activating properties.

In one aspect, the present invention provides, a population of genetically engineered T cells, comprising a chimeric antigen receptor (CAR) that recognizes a cancer associated antigen and a TGFβ signaling pathway modulator.

In some embodiments, the population of cells comprises a CAR that recognizes an antigen selected from the group consisting of ADGRE2, CLEC12, CAIX, CEA, CD5, CD7, CD10, CD19, CD20, CD22, CD30, CD33, CD34, CD38, CD41, CD44, CD49f, CD56, CD74, CD133, CD138, a cytomegalovirus (CMV) infected cell antigen, CEACAM 5, Claudin 18.2, EGP-2, EGP-40, EpCAM, erb-B2,3,4, FBP, Fetal acetylcholine receptor, folate receptor-a, GCC (also known as GUCY2C), GD2, GD3, HER-2, hTERT, IL-13R-a2, x-light chain, KDR, LeY, LI cell adhesion molecule, MAGE-AI, MUC1, MUC13, Mesothelin, NKG2D ligands, NY-ES0-1, oncofetal antigen (h5T4), PSCA, PSMA, PTK7, ROR1, TAG-72, TROP2, VEGF-R2, and WT-1.

In some embodiments, the population of cells comprises a is a CD19 CAR or a GCC CAR.

In some embodiments, the population of cells comprises a TGFβ signaling pathway modulator that binds TGFβ or a TGFβ receptor.

In some embodiments, the population of cells comprises a TGFβ signaling pathway modulator comprising an amino acid sequence selected from Table 1.

In some embodiments, the population of cells are autologous.

In some embodiments, the population of cells are allogeneic.

In some embodiments, the population of cells are primary cells. In some embodiments, the population of cells are derived from induced pluripotent stem cells (iPSCs).

In some embodiments, the population of cells are genetically modified using a vector comprising a first nucleic acid encoding a CAR polypeptide and a second nucleic acid encoding a TGFβ signaling pathway modulator.

In some embodiments, the population of cells are genetically modified using two vectors, first vector comprising a nucleic acid encoding a CAR polypeptide and a second vector comprising a nucleic acid encoding a TGFβ signaling pathway modulator.

In some embodiments, the population of cells are genetically modified using Crispr. In some embodiments, the population of cells are genetically modified using retroviral transduction (including g-retroviral), lentiviral transduction, transposon amd transposases (Sleeping Beauty and PiggyBac systems), messenger RNA transfer-mediated gene expression, gene editing (gene insertion or gene deletion/disruption), CRISPR-Cas9, ZFN (zinc finger nuclease), or TALEN (transcription activator like effector nuclease) systems.

In some embodiments, the population of cells comprises a CAR comprising an intracellular signaling domain selected from the group consisting of CD3ζ-chain, CD97, 2B4, GDI la-CD18, CD2, ICOS, CD27, CD154, CDS, OX40, 4-1BB, DAP10, DAP12, CD28 signaling domain, or combinations and variations thereof.

In some embodiments, the population of cells comprises a CAR comprising a transmembrane domain derived from a transmembrane domain selected from the group consisting of CD3, CD8, CD28, OX40, CD27, 4-1BB, DAP10, DAP12 or combinations thereof.

In one aspect, the present invention provides a vector comprising a first nucleic acid encoding a CAR polypeptide and a second nucleic acid encoding a TGFβ signaling pathway modulator.

In some embodiments, the vector comprising a first nucleic acid encoding a CAR polypeptide and a second nucleic acid encoding a TGFβ signaling pathway modulator comprises an internal ribosomal entry site.

In some embodiments, the vector further comprising a 2A ribosome sequence.

In one aspect the present invention provides an immune cell modified with a vector comprising a first nucleic acid encoding a CAR polypeptide and a second nucleic acid encoding a TGFβ signaling pathway modulator.

In some embodiments, the immune cell is a T-cell.

In one aspect, the present invention provides a method of modulating an immune response in a host, the method comprising administering to the host a population of genetically engineered T cells, comprising a chimeric antigen receptor (CAR) that recognizes a cancer associated antigen and a TGFβ signaling pathway modulator, wherein the modulation of immune response comprises one or more of the following by host immune cells: increase in IFNγ production; increase in IL-2 production; increase in antigen presentation; and increase in proliferation.

In one aspect, the present invention provides a pharmaceutical composition comprising a population of genetically engineered T cells, comprising a chimeric antigen receptor (CAR) that recognizes a cancer associated antigen and a TGFβ signaling pathway modulator.

In one aspect, the present invention provides a method of treating or preventing cancer in a subject in need thereof, the method comprising administering to the subject an effective amount of a population of genetically engineered T cells, comprising a chimeric antigen receptor (CAR) that recognizes a cancer associated antigen and a TGFβ signaling pathway modulator.

In some embodiments, the cancer is selected from the group consisting of leukemia, acute leukemia, acute lymphocytic leukemia, acute myelocytic leukemia, acute myeloblastic leukemia, acute promyelocyte leukemia, acute myelomonocytic leukemia, acute monocytic leukemia, acute erythroleukemia, chronic leukemia, chronic myelocytic leukemia, multiple myeloma, chronic lymphocytic leukemia, polycythemia vera, lymphoma, Hodgkin's lymphoma, non-Hodgkin's lymphoma, Waldenstrom's macroglobulinemia, heavy chain disease, solid tumors, sarcoma, carcinoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, hepatocellular carcinoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, colorectal carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, retinoblastoma, and metastasis thereof.

In one aspect, the present invention provides an immune modulating system comprising a nucleic acid sequence encoding a chimeric antigen receptor (CAR); and a nucleic acid sequence encoding a polypeptide that modulates TGF-b signaling (e.g., TGFβ signaling modulator).

In some embodiments, the polypeptide that modulates TGF-b signaling comprises a variable heavy chain (vH).

In some embodiments, the polypeptide that modulates TGF-b signaling comprises a variable heavy chain (vH) and variable light chain (vL).

In some embodiments, the polypeptide that modulates TGF-b signaling comprises an antigen binding molecule selected from the group consisting of an IgA antibody, IgG antibody, IgE antibody, IgM antibody, bi- or multi-specific antibody, Fab fragment, Fab′ fragment, F(ab′)2 fragment, Fd′ fragment, Fd fragment, isolated CDRs or sets thereof; single-chain variable fragment (scFv), polypeptide-Fc fusion, single domain antibody (sdAb), camelid antibody; masked antibody, Small Modular ImmunoPharmaceuticals (“SMIPsTM”), single chain, Tandem diabody, VHHs, Anticalin, Nanobody, humabody, minibodies, BiTE, ankyrin repeat protein, DARPIN, Avimer, DART, TCR-like antibody, Adnectin, Affilin, Trans-body; Affibody, TrimerX, MicroProtein, Fynomer, Centyrin; and KALBITOR, or fragments thereof.

In some embodiments, the polypeptide that modulates TGF-b signaling comprises a single-chain variable fragment (scFv). In some embodiments, the polypeptide that modulates TGF-b signaling comprises a single domain antibody (sdAb) In some embodiments, the polypeptide that modulates TGF-b signaling comprises a heavy chain only antibody.

In some embodiments, the polypeptide that modulates TGF-b signaling comprises an amino acid sequence selected from Table 1.

In some embodiments, the polypeptide that modulates TGF-b signaling comprises a dimeric antigen binding agent.

In some embodiments, the polypeptide that modulates TGF-b signaling binds to TGF-b.

In some embodiments, the polypeptide that modulates TGF-b signaling binds to a TGF-b receptor.

In some embodiments, the polypeptide that modulates TGF-b signaling binds to TGF-b receptor 2 (TGF-bR2).

In some embodiments, the polypeptide that modulates TGF-b signaling comprises TGF-b receptor 2 (TGF-bR2) or a fragment thereof.

In some embodiments, the polypeptide that modulates TGF-b signaling comprises an extracellular domain of TGF-bR2 (TGF-bR2).

In some embodiments, the CAR binds to an antigen selected from the group consisting of ADGRE2, CLEC12, CAIX, CEA, CD5, CD7, CD10, CD19, CD20, CD22, CD30, CD33, CD34, CD38, CD41, CD44, CD49f, CD56, CD74, CD133, CD138, a cytomegalovirus (CMV) infected cell antigen, CEACAM 5, Claudin, 18.2, EGP-2, EGP-40, EpCAM, erb-B2,3,4, FBP, Fetal acetylcholine receptor, folate receptor-a, GCC (also known as GUCY2C), GD2, GD3, HER-2, hTERT, IL-13R-a2, x-light chain, KDR, LeY, LI cell adhesion molecule, MAGE-AI, MUC1, MUC13, Mesothelin, NKG2D ligands, NY-ES0-1, oncofetal antigen (h5T4), PSCA, PSMA, PTK7 ROR1, TAG-72, TROP2, VEGF-R2, and WT-1.

In some embodiments, the CAR binds to CD19 or GCC.

In some embodiments, the CAR comprises an intracellular signaling domain selected from the group consisting of CD3ζ-chain, CD97, 2B4, GDI la-CD18, CD2, ICOS, CD27, CD154, CDS, OX40, 4-1BB, DAP10, DAP12, CD28 signaling domain, or combinations and variations thereof.

The immune modulating system of any one of the preceding claims, wherein the CAR comprises a transmembrane domain derived from a transmembrane domain selected from the group consisting of CD3, CD8, CD28, OX40, CD27, 4-1BB, DAP10, DAP12 or combinations thereof.

In certain embodiments, the modified CD3z polypeptide lacks all or part of immunoreceptor tyrosine-based activation motifs (ITAMs), wherein the ITAMs are ITAM1, ITAM2, and ITAM3. In certain embodiments, the modified CD3z polypeptide further lacks all or part of basic-rich stretch (BRS) regions, wherein the BRS regions are BRS1, BRS2, and BRS3.

In one aspect the present invention provides a nucleic acid comprising the immune modulating system described herein, wherein the sequence encoding a chimeric antigen receptor (CAR); and the sequence encoding a polypeptide that modulates TGF-b signaling are present on a single construct.

In some embodiments, the sequence encoding a chimeric antigen receptor (CAR); and the sequence encoding a polypeptide that modulates TGF-b signaling a present on different constructs.

In one aspect, the present invention provides a vector comprising the nucleic acid encoding the immune modulating system described herein.

In some embodiments, the vector comprises an Internal Ribosomal Entry Site (IRES).

In some embodiments, the vector comprises a 2A ribosome sequence. In some embodiments, the 2A ribosome sequence is P2A or T2A.

In one aspect, the present invention provides an immunoresponsive cell comprising the immune modulating system described herein.

In one aspect, the present invention provides an immunoresponsive cell comprising: a targeting agent with specificity to a tumor associated antigen or a stress ligand and a nucleic acid encoding a recombinant polypeptide that modulates TGF-b signaling.

In some embodiments, the targeting agent specifically binds a stress ligand selected from the group consisting of MIC-A, MIC-B, ULBP1-6;

In one aspect, the present invention provides an immunoresponsive cell comprising: a chimeric antigen receptor (CAR); and a nucleic acid encoding a recombinant polypeptide that modulates TGF-b signaling.

In some embodiments, the CAR and the nucleic acid encoding a polypeptide that modulates TGF-b signaling are provided on the same polynucleotide.

In some embodiments, the CAR and the nucleic acid encoding a polypeptide that modulates TGF-b signaling are provided on separate polynucleotides.

In some embodiments, the recombinant polypeptide that modulates TGF-b signaling is secreted from the cell.

In some embodiments, the recombinant polypeptide that modulates TGF-b signaling comprises a variable heavy chain (vH).

In some embodiments, the recombinant polypeptide that modulates TGF-b signaling comprises a variable heavy chain (vH) and variable light chain (vL).

In some embodiments, the recombinant polypeptide that modulates TGF-b signaling comprises a single-chain variable fragment (scFv).

In some embodiments, the recombinant polypeptide that modulates TGF-b signaling comprises a dimeric antigen binding agent.

In some embodiments, the polypeptide that modulates TGF-b signaling binds to TGF-b.

In some embodiments, the recombinant polypeptide that modulates TGF-b signaling comprises TGF-b receptor 2 (TGF-bR2) or a fragment thereof.

In some embodiments, the recombinant polypeptide that modulates TGF-b signaling comprises an extracellular domain of TGF-bR2.

In some embodiments, the polypeptide that modulates TGF-b signaling binds to a TGF-b receptor.

In some embodiments, the polypeptide that modulates TGF-b signaling binds to TGF-b receptor 2 (TGF-bR2).

In some embodiments, the immunoresponsive cell comprises a CAR expressed from a vector, an engineered mRNA, or integrated into the host cell chromosome. In some embodiments, the sequence encoding the CAR is integrated into the host cell chromosome using an endonuclease. In some embodiments, the sequence encoding the CAR is integrated into the host cell chromosome using Crispr/Cas9, Cas12a, or Cas13.

In some embodiments, the immunoresponsive cell comprises a recombinant polypeptide that modulates TGF-b signaling is expressed from a vector, an engineered mRNA, or integrated into the host cell chromosome. In some embodiments, the sequence encoding the polypeptide that modulates TGF-b signaling is integrated into the host cell chromosome using Crispr/Cas9, Cas12a, or Cas13.

In some embodiments, the immunoresponsive cell is selected from the group consisting of a T cell, a Natural Killer (NK) cell, a Natural Killer (NK) T cell, a gamma delta T cell, a cytotoxic T lymphocyte (CTL), a regulatory T cell, a human embryonic stem cell, B cell, macrophage, and a pluripotent stem cell from which lymphoid cells may be differentiated (e.g., an NK or T cell derived from an iPSC).

In some embodiments, the immunoresponsive cell is an engineered autologous cell. In some embodiments, the immunoresponsive cell is an engineered allogeneic cell.

In some embodiments, the immunoresponsive cell comprises a CAR that binds to a tumor antigen selected from the group consisting of ADGRE2, CLEC12, CAIX, CEA, CD5, CD7, CD10, CD19, CD20, CD22, CD30, CD33, CD34, CD38, CD41, CD44, CD49f, CD56, CD74, CD133, CD138, a cytomegalovirus (CMV) infected cell antigen, CEACAM 5, Claudin, 18.2, EGP-2, EGP-40, EpCAM, erb-B2,3,4, FBP, Fetal acetylcholine receptor, folate receptor-a, GCC (also known as GUCY2C), GD2, GD3, HER-2, hTERT, IL-13R-a2, x-light chain, KDR, LeY, LI cell adhesion molecule, MAGE-AI, MUC1, MUC13, Mesothelin, NKG2D ligands, NY-ES0-1, oncofetal antigen (h5T4), PSCA, PSMA, PTK7, ROR1, TAG-72, TROP2, VEGF-R2, and WT-1.

In some embodiments, the CAR binds to CD19 or GCC. In some embodiments, the CAR binds to GCC.

In some embodiments, the CAR comprises an intracellular signaling domain derived from CD3ζ, CD97, 2B4, GDI la-CD18, CD2, ICOS, CD27, CD154, CDS, OX40, 4-1BB, DAP10, DAP12, CD28 signaling domain, or combinations and variations thereof.

In some embodiments, the CAR comprises a transmembrane domain derived from a transmembrane domain selected from the group consisting of CD3, CD8, CD28, OX40, CD27, 4-1BB, DAP10, DAP12 or combinations thereof.

In certain embodiments, the modified CD3z polypeptide lacks all or part of immunoreceptor tyrosine-based activation motifs (ITAMs), wherein the ITAMs are ITAM1, ITAM2, and ITAM3. In certain embodiments, the modified CD3z polypeptide further lacks all or part of basic-rich stretch (BRS) regions, wherein the BRS regions are BRS1, BRS2, and BRS3.

In some embodiments, immunoresponsive cell comprises a chimeric costimulatory receptor (CCR). In some embodiments, the CAR comprises a co-stimulatory domain. In some embodiments, the CAR does not comprise an intracellular signaling domain. In some embodiments, the CAR does not comprise a CD3z domain.

In some embodiments, the recombinant polypeptide that modulates TGF-b signaling enhances an immune response of the immunoresponsive cell.

In one aspect, the present invention provides a pharmaceutical composition comprising an effective amount of the immune modulating system described herein.

In one aspect, the present invention provides a pharmaceutical composition comprising an effective amount of the nucleic acid sequence encoding the immune modulating system described herein.

In one aspect, the present invention provides a pharmaceutical composition comprising an effective amount of the vector encoding the immune modulating system described herein.

In one aspect, the present invention provides a pharmaceutical composition comprising an effective amount of the immunoresponsive cell described herein.

In some embodiments, the pharmaceutical composition further comprises a pharmaceutically acceptable excipient.

In one aspect, the present invention provides a kit for treating a cancer, the kit comprising an immunoresponsive cell comprising a chimeric antigen receptor (CAR); and a nucleic acid encoding a recombinant polypeptide that modulates TGF-b signaling.

In some embodiments, the kit comprises the nucleic acid or vector encoding the immune modulating system described herein.

In one aspect, the present invention provides a method of treating or preventing cancer or metastasis thereof in a subject, the method comprising administering an effective amount of an immunoresponsive cell comprising a chimeric antigen receptor (CAR); and a nucleic acid encoding a recombinant polypeptide that modulates TGF-b signaling.

In some embodiments, the compostions described herein can be used for treating a hematopoietic cancer. In other embodiments, the compositions described herein can be used for treating a solid tumor cancer.

In some embodiments, the cancer is selected from the group consisting of leukemia, acute leukemia, acute lymphocytic leukemia, acute myelocytic leukemia, acute myeloblastic leukemia, acute promyelocyte leukemia, acute myelomonocytic leukemia, acute monocytic leukemia, acute erythroleukemia, chronic leukemia, chronic myelocytic leukemia, multiple myeloma, chronic lymphocytic leukemia, polycythemia vera, lymphoma, Hodgkin's lymphoma, non-Hodgkin's lymphoma, Waldenstrom's macroglobulinemia, heavy chain disease, solid tumors, sarcoma, carcinoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, hepatocellular carcinoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, colorectal carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, and retinoblastoma.

In some embodiments, the method further comprises administering a second therapeutic agent to the subject.

In some embodiments, the second therapeutic agent is administered systemically to the subject.

In some embodiments, the second therapeutic agent is administered separately from the CAR and the nucleic acid encoding a recombinant polypeptide that modulates TGF-b signaling

In some embodiments, the second therapeutic agent targets PD1/PD-L1, CXCR2, and/or IL-15.

In some embodiments, the second therapeutic agent is a PD1/PD-L1 inhibitor.

In one aspect, the present invention provides a method of modulating the activity of an immune cell, the method comprising administering a nucleic acid encoding a chimeric antigen receptor (CAR); and a nucleic acid encoding a recombinant polypeptide that modulates TGF-b signaling.

In one aspect, the present invention provides a method of modulating the activity of a chimeric antigen receptor (CAR), the method comprising administering a nucleic acid encoding a chimeric antigen receptor (CAR); and a nucleic acid encoding a recombinant polypeptide that modulates TGF-b signaling.

In one aspect, the present invention provides a method of reducing tumor burden in a subject, the method comprising administering an effective amount of the immune modulating system comprising the nucleic acid, the vector, or the immunoresponsive cell described herein.

In some embodiments, the method reduces the number of tumor cells. In some embodiments, the method reduces tumor size. In some embodiments, the method eradicates the tumor in the subject.

In one aspect, the present invention provides a method of increasing immune-activating cytokine production in response to a cancer cell in a subject, comprising administering to the subject, the immune modulating system comprising the nucleic acid, the vector, or the immunoresponsive cell described herein.

In one aspect, the present invention provides a method for producing an antigen-specific immunoresponsive cell, the method comprising introducing into the immunoresponsive cell a nucleic acid sequence that encodes a chimeric antigen receptor (CAR); and a nucleic acid that encodes a recombinant polypeptide that modulates TGF-b signaling.

It is to be understood that both the foregoing general description and the following detailed description are exemplary and explanatory only and are not restrictive of the invention, as claimed.

### BRIEF DESCRIPTION OF THE DRAWING

The Drawing included herein, which is comprised of the following Figures, is for illustration purposes only not for limitation.

FIG. 1A-1E demonstrates exemplary expression of CAR and TGF-β signaling modulators in immunoresponsive cells (e.g., transduced T cells). FIG. 1A illustrates the population of lymphocytes, FIG. 1B illustrates singlets, FIG. 1C depicts populations of live CD3+ cells and FIG. 1D shows exemplary flow cytometry results evaluating CAR expression in armored human CAR-T cells expressing TGF-β. FIG. 1E depicts a bar graph showing transduction efficiencies as % live cells positive for CAR staining using unarmored cells transduced with a CD19 CAR only, and CD19 CAR-T cells armored with TGFb scFv VH-VL1 (SEQ ID NO: 1), TGFb scFv VH-VL2 (SEQ ID NO: 2), TGFb scFv VL-VH (SEQ ID NO: 3), TGFbR2 scFv VH-VL (SEQ ID NO: 4), TGFbR2 scFv VL-VH (SEQ ID NO: 5), mTGFbR2 VH1 (SEQ ID NO: 6) and hTGFbR2 VH1 (SEQ ID NO: 8) or untransduced cells.

FIG. 2A-2B are graphs demonstrating exemplary in vitro killing assay results of immunoresponsive cells co-expressing an anti-CD19 CAR and a TGF-β signaling modulator against CD19+ Raji cells (FIG. 2A) and CD19ko Raji cells (FIG. 2B) using effector relative to target of unarmored cells transduced with a CD19 CAR only, and CD19 CAR-T cells armored with TGFb scFv VH-VL1 (SEQ ID NO: 1), TGFb scFv VH-VL2 (SEQ ID NO: 2), TGFb scFv VL-VH (SEQ ID NO: 3), TGFbR2 scFv VH-VL (SEQ ID NO: 4), TGFbR2 scFv VL-VH (SEQ ID NO: 5), mTGFbR2 VH1 (SEQ ID NO: 6) and hTGFbR2 VH1 (SEQ ID NO: 8) or untransduced cells.

FIG. 3A depicts a bar graph depicting exemplary ELISA results demonstrating secretion of TGF-β binders and FIG. 3B depicts a bar graph depicting exemplary ELISA results demonstrating secretion of TGFβR2 binders by human CAR-T cells and their ability to bind to their cognate antigen.

FIG. 4 depicts a bar graph depicting exemplary results of a luciferase assay evaluating the inhibition of TGF-β signaling by supernatant from CAR-T cells secreting constructs TGFb scFv VH-VL1 (SEQ ID NO: 1), TGFb scFv VH-VL2 (SEQ ID NO: 2), TGFb scFv VL-VH (SEQ ID NO: 3), TGFbR2 scFv VH-VL (SEQ ID NO: 4), TGFbR2 scFv VL-VH (SEQ ID NO: 5), mTGFbR2 VH1 (SEQ ID NO: 6) and hTGFbR2 VH1 (SEQ ID NO: 8).

FIG. 5A shows a bar graph depicting exemplary results of a luciferase assay evaluating the inhibition of TGF-β signaling by supernatant from CAR-T cells secreting TGFb-scFv VH-VL1 G4S dimer (SEQ ID NO: 17), TGFb-scFv VH-VL1 2×G4S dimer (SEQ ID NO: 18) TGFb-scFv VH-VL1 minibody (SEQ ID NO: 21), TGFb-scFv VH-VL1 minibody+hinge (SEQ ID NO: 19). FIG. 5B shows a schematic of exemplary TGF-β modulators designed and screened using a luciferase reporter assay for the secretion of multimeric binders against TGF-β. FIG. 5C shows a schematic of exemplary TGF-β modulators comprising VHH binding domains.

FIG. 6A shows a bar graph depicting exemplary results of the luciferase assay evaluating the relative blocking activity of armored CAR T cells co-expressing monomeric TGFb scFv VH-VL1 (SEQ ID NO: 1) and dimeric TGFb-scFv VH-VL1 G4S dimer (SEQ ID NO: 17) binders compared to unarmored cells expressing a CAR alone.

FIG. 6B shows a bar graph depicting exemplary results of a luciferase assay used to evaluate the relative blocking activity of TGFβR2 VHH and scFv monomer and dimer constructs. Unarmored CAR-T cells, mTGFbR2 VH2 monomer, mTGFbR2 VH2 G4S dimer, mTGFbR2 VH2 G4S trimer, hTGFbR2 VH2 monomer, hTGFbR2 VH2 G4S dimer, hTGFbR2 VH3 monomer, hTGFbR2 VH3 G4S dimer, hTGFbR2 scFv VH-VL monomer, hTGFbR2 scFv VH-VL G4S dimer.

FIG. 7A and FIG. 7B depict bar graphs depicting exemplary ELISA results demonstrating that exemplary TGFb modulators bind to human TGFbR2 (FIG. 7A) but not to mouse TGFbR2 (FIG. 7B). Unarmored CAR-T cells, mTGFbR2 VH2 monomer, mTGFbR2 VH2 G4S dimer, mTGFbR2 VH2 G4S trimer, hTGFbR2 VH2 monomer, hTGFbR2 VH2 G4S dimer, hTGFbR2 VH3 monomer, hTGFbR2 VH3 G4S dimer, hTGFbR2 scFv VH-VL monomer, hTGFbR2 scFv VH-VL G4S dimer

FIG. 8A shows an exemplary injection timeline to evaluate tumor growth of EMT6-hCD19-Fluc tumor cells as described in Example 6. FIG. 8B shows exemplary tumor volume over time in mice that received CAR-T cells secreting a TGF-β binder relative to unarmored CAR-T or untransduced CAR-T cells. FIG. 8C demonstrates exemplary liver metastasis in mice treated with CAR-T cells that secrete TGF-β binders relative to unarmored or untransduced CAR-T cells. FIG. 8D demonstrates exemplary lung metastasis in mice treated with CAR-T cells that secrete TGF-β binders relative to unarmored or untransduced CAR-T cells. FIG. 8E demonstrates exemplary imaging results of luciferase expressing tumor cells in liver and lung tissues.

FIG. 9A and FIG. 9B depict bar graphs depicting exemplary results of a SBE-Luc TGF-b reporter assay comparing supernatant from armored mouse CAR-T secreting different TGF-b ligand traps (TGF-b scFv VH-VL1 to TGFbR2 ECD monomers, homodimers (FIG. 9A) and heterodimers (FIG. 9B)) to unarmored CAR-T cells.

FIG. 10A shows an exemplary injection timeline to evaluate tumor growth of EMT6-hCD19-Fluc tumor cells. FIG. 10B shows exemplary tumor volume over time in mice that received untransduced T cells or unarmored CAR-T cells (CAR-T cells that do not co-express a TGFβ signaling modulator). FIG. 10C shows exemplary tumor volume over time in mice that received armored CAR-T cells co-expressing TGFbRT+2ECD dimer or unarmored CAR-T cells (CAR-T cells that do not co-express a TGFβ signaling modulator). FIG. 10D shows exemplary tumor volume over time in mice that received systemic anti-TGFb antibody (1D11) or unarmored CAR-T cells (CAR-T cells that do not co-express a TGFβ signaling modulator).

FIG. 11 depicts a graph demonstrating exemplary tumor volume in mice developed from MC38 cells expressing CD19 over time. Mice received untransduced T cells, unarmored antiCD19 CAR-T cells or CAR-T cells secreting an inhibitory binder against TGF-b (TGF-b scFv VH-VL1).

FIG. 12 depicts a graph showing exemplary RNA Seq analysis demonstrating enhanced activation of the host immune response by CAR-T cells secreting a binder against TGF-b (TGF-b scFv VH-VL1 (SEQ ID NO: 1)).

FIG. 13 shows exemplary biomarker scores for tumor infiltrating T cells (CD3d+, CD3e+, CD3g+), CD8+ T cells (CD8a+) and cytotoxic T cells (GzmB+) in tumors from mice that received TGF-b scFv VH-VL1 (SEQ ID NO: 1) secreting CAR-T cells.

FIG. 14 shows exemplary single-sample Gene Set Enrichment Analysis (GSEA), enrichment scores demonstrating increased T cell signatures and IFNg signatures in tumors from mice that received TGF-b scFv VH-VL1 (SEQ ID NO: 1) secreting CAR-T cells.

FIG. 15 shows exemplary surface marker analysis of including TCRa/b, CD8a, CD4, CD25, CD62L, CD11b, Gr1, CD11c, CD45.1 and CD45, in mice receiving untransduced control T cells, unarmored CD19 CAR-T cells, or anti-TGF-b scFv VH-VL1 (SEQ ID NO: 1) monomer secreting CAR-T cells.

FIG. 16A and FIG. 16B are graphs depicting exemplary in vivo analysis in a GSU xenograft model demonstrating improved function of human GCC-CAR-T cells armored with anti-TGF-b or anti-TGFbR2 blocking antibodies.

FIG. 17A-17D demonstrate tumor and/or plasma concentrations of TGFb modulators secreted by anti-GCC CAR-T cells co-expressing TGF-b scFv VH-VL1 and TGFbR2 VHH determined using an anti-Flag immune capture LC/MS assay.

FIG. 18A-18C are graphs depicting exemplary in vitro killing assay results in HT29-GCC positive cells using unarmored anti-GCC CAR-T cells, anti-TGFbR2 VHH monomer armored anti-GCC CAR-T cells and anti-TGFbR2 VHH dimer armored anti-GCC CAR-T cells in the absence of TGFb (FIG. 18A) and in the presence of TGFb (FIG. 18B). FIG. 18C shows CAR T cell proliferation in the presence and absence of TGFb.

FIG. 19 depicts exemplary flow cytometry results demonstrating PD-1/Lag3 expression on cells following repeat antigen stimulation.

FIG. 20A-20C depict (s.c.) xenograft models of GCC expressing cells GSU (FIG. 20A), HT55 (FIG. 20B), and MDA-MB-231-FP4 Luc (FIG. 20C) treated with armored and unarmored CAR-T cells and CAR-T cells expressing dominant negative TGFbR2 (dnTGFbR2).

FIG. 21A-21C shows exemplary results of an HT55 liver metastasis model treated with armored anti-GCC CAR T cells.

FIG. 22A shows CAR-T cells counted by flow cytometry and FACS phenotyping performed at indicated time-points. FIG. 22B shows percent cytotoxicity in anti-Msln CAR-T cells, co-expressing a CAR against Msln together with a TGFβ modulator (e.g., TGFβR2-VH or dnTGFbR2) or a control VH against GFP (Msln-control VH).

### DEFINITIONS

In order for the present invention to be more readily understood, certain terms are first defined below. Additional definitions for the following terms and other terms are set forth through the specification.

As used in this specification and the appended claims, the singular forms “a”, “an”, and “the” include plural references unless the context clearly dictates otherwise. Thus for example, a reference to “a method” includes one or more methods, and/or steps of the type described herein and/or which will become apparent to those persons skilled in the art upon reading this disclosure and so forth.

Administer: As used herein, “administering” a composition to a subject means to give, apply or bring the composition into contact with the subject. Administration can be accomplished by any of a number of routes, such as, for example, topical, oral, subcutaneous, intramuscular, intraperitoneal, intravenous, intrathecal, and intradermal.

Adoptive Cell Therapy: As used herein interchangeably, the terms “adoptive cell therapy” or “adoptive cell transfer” or “cell therapy” or “ACT” refer to the transfer of cells, for example, a population of genetically modified cells described herein, into a patient in need thereof. The cells can be derived and propagated from the patient in need thereof (i.e., autologous cells) or could have been obtained from a non-patient donor (i.e., allogeneic cells). In some embodiments, the cell is an immune cell, such as a lymphocyte, modified to express a CAR and a TGFβ signaling pathway modulator, as describe herein (e.g., a TGFβ armored CAR-T cell). Various cell types can be used for ACT including but not limited to, natural killer (NK) cells, T cells, CD8+ cells, CD4+ cells, gamma delta T-cells, regulatory T-cells, induced pluripotent stem cells (iPSCs), iPSC derived T cells, iPSC derived NK cells, hematopoietic stem cells (HSCs), mesenchymal stem cells (MSCs) and peripheral blood mononuclear cells.

Affinity: As used herein, the term “affinity” refers to the characteristics of a binding interaction between a binding moiety (e.g., an antigen binding agent (e.g., variable domain described herein) and a target (e.g., an antigen (e.g., TGFB or TGFBR) and that indicates the strength of the binding interaction. In some embodiments, the measure of affinity is expressed as a dissociation constant (K_{D}). The binding affinity of an antigen binding protein to its target may be determined by equilibrium methods (e.g., enzyme-linked immunoabsorbent assay (ELISA) or radioimmunoassay (RIA)), kinetics (e.g. BIACORE™ analysis), or other methods known in the art.

Avidity: As used herein, the term “avidity” is the sum total of the strength of binding of two molecules to one another at multiple sites, e.g. taking into account the valency of the interaction.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Autologous: As used herein, the term “autologous” is refers to any material derived from the same individual to whom it is later to be re-introduced into the individual.

Allogeneic: “Allogeneic” as used herein, refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently different genetically to interact antigenically.

Antibody or Antigen Binding Agent: As used herein, the term “antibody” or “antigen binding agent” refers to a polypeptide that includes canonical immunoglobulin sequence elements sufficient to confer specific binding to a particular target antigen. Those skilled in the art will appreciate that the terms may be used herein interchangeably. In some embodiments, as used herein, the term “antibody” or “antigen binding agent” also refers to an “antibody fragment” or “antibody fragments”, which includes a portion of an intact antibody, such as, for example, the antigen-binding or variable region of an antibody. Examples of “antibody fragments” include Fab, Fab′, F(ab′)2, and Fv fragments; triabodies; tetrabodies; linear antibodies; single-chain antibody molecules; and CDR-containing moieties included in multi-specific antibodies formed from antibody fragments. Those skilled in the art will appreciate that the term “antibody fragment” does not imply and is not restricted to any particular mode of generation. An antibody fragment may be produced through use of any appropriate methodology, including but not limited to cleavage of an intact antibody, chemical synthesis, recombinant production, etc. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (V_{H}) domain (located at the tips of the Y structure), followed by three constant domains: C_{H}1, C_{H}2, and the carboxy-terminal C_{H}3 (located at the base of the Y's stem). A short region, known as the “switch”, connects the heavy chain variable and constant regions. The “hinge” connects C_{H}2 and C_{H}3 domains to the rest of the antibody. Two disulfide bonds in this hinge region connect the two heavy chain polypeptides to one another in an intact antibody. Each light chain is comprised of two domains—an amino-terminal variable (V_{L}) domain, followed by a carboxy-terminal constant (C_{L}) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the C_{H}2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. Amino acid sequence comparisons among antibody polypeptide chains have defined two light chain (κ and λ) classes, several heavy chain (e.g., μ, γ, α, ε, δ) classes, and certain heavy chain subclasses (α1, α2, γ1, γ2, γ3, and γ4). Antibody classes (IgA [including IgA1, IgA2], IgD, IgE, IgG [including IgG1, IgG2, IgG3, and IgG4], and IgM) are defined based on the class of the utilized heavy chain sequences.

For purposes of the present invention, in certain embodiments, any polypeptide or complex of polypeptides that includes sufficient immunoglobulin domain sequences as found in natural antibodies can be referred to and/or used as an “antibody” or “antigen binding agent”, whether such polypeptide is naturally produced (e.g., generated by an organism reacting to an antigen), or produced by recombinant engineering, chemical synthesis, or other artificial system or methodology. In some embodiments, an antibody is monoclonal; in some embodiments, an antibody is polyclonal. In some embodiments, an antibody has constant region sequences that are characteristic of mouse, rabbit, primate, or human antibodies. In some embodiments, an antibody sequence elements are humanized, primatized, chimeric, etc., as is known in the art. Moreover, the term “antibody” or “antigen binding agent” as used herein, will be understood to encompass (unless otherwise stated or clear from context) can refer in appropriate embodiments to any of the art-known or developed constructs or formats for capturing antibody structural and functional features in alternative presentation. For example, in some embodiments, the terms can refer to bi- or other multi-specific (e.g., zybodies, etc.) antibodies, Small Modular ImmunoPharmaceuticals (“SMIPs™”), single chain antibodies, camelid antibodies, and/or antibody fragments. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc.], or other pendant group [e.g., poly-ethylene glycol, etc.]).

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). It is understood that when the term “about” or “approximately” is used to modify a stated reference value, the stated reference value itself is covered along with values that are near the stated reference value on either side of the stated reference value.

Armored CAR-T cell: As used herein, the term “armored CAR cells” or “armored CAR-T cells” refers to genetically engineered cells with the ability to evade tumor immunosuppression and tumor-induced CAR-T hypofunction. In some embodiments, an armored CAR T cell comprises a chimeric antigen receptor (CAR) that recognizes a cancer associated antigen and a TGFβ signaling pathway modulator.

Complementarity Determining Region (CDR): A “CDR” of a variable domain are amino acid residues within the variable region that are identified in accordance with the definitions of the Kabat, Chothia, the accumulation of both Kabat and Chothia, AbM, contact, and/or conformational definitions or any method of CDR determination well known in the art. Antibody CDRs may be identified as the hypervariable regions originally defined by Kabat et al. See, e.g., Kabat et al., 1992, Sequences of Proteins of Immunological Interest, 5th ed., Public Health Service, NIH, Washington D.C. The positions of the CDRs may also be identified as the structural loop structures originally described by Chothia and others. See, e.g., Chothia et al., Nature 342:877-883, 1989. Other approaches to CDR identification include the “AbM definition,” which is a compromise between Kabat and Chothia and is derived using Oxford Molecular's AbM antibody modeling software (now Accelrys®), or the “contact definition” of CDRs based on observed antigen contacts, set forth in MacCallum et al., J. Mol. Biol., 262:732-745, 1996. In another approach, referred to herein as the “conformational definition” of CDRs, the positions of the CDRs may be identified as the residues that make enthalpic contributions to antigen binding. See, e.g., Makabe et al., Journal of Biological Chemistry, 283: 1 156-1166, 2008. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues or even entire CDRs do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, AbM, contact, and/or conformational definitions.

Antibody-dependent cell-mediated cytotoxicity or ADCC refers to a form of cytotoxicity in which secreted Ig bound onto Fc receptors (FcRs) present on certain cytotoxic cells (e.g., natural killer (NK) cells, neutrophils and macrophages) enable these cytotoxic effector cells to bind specifically to an antigen-bearing target cell and subsequently kill the target cell with cytotoxins. The antibodies “arm” the cytotoxic cells and are required for killing of the target cell by this mechanism. The primary cells for mediating ADCC, NK cells, express FcγRIII only, whereas monocytes express FcγRI, FcγRII and FcγRIII. Fc expression on hematopoietic cells is summarized in Table 3 on page 464 of Ravetch and Kinet, Annu. Rev. Immunol. 9: 457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and natural killer (NK) cells. Alternatively, or additionally, ADCC activity of the molecule of interest may be assessed in vivo, e.g., in an animal model such as that disclosed in Clynes et al., PNAS USA 95: 652-656 (1998).

Antigen: As used herein, the term “antigen”, refers to an agent that elicits an immune response; and/or an agent that is bound by a T cell receptor (e.g., when presented by an MHC molecule) or to an antibody (e.g., produced by a B cell) when exposed or administered to an organism. In some embodiments, an antigen elicits a humoral response (e.g., including production of antigen-specific antibodies) in an organism; alternatively or additionally, in some embodiments, an antigen elicits a cellular response (e.g., involving T-cells whose receptors specifically interact with the antigen) in an organism. It will be appreciated by those skilled in the art that a particular antigen may elicit an immune response in one or several members of a target organism (e.g., mice, rabbits, primates, humans), but not in all members of the target organism species. In some embodiments, an antigen elicits an immune response in at least about 25%, 30%, 35%, 40%, 45%, 50%, 55%, 60%, 65%, 70%7, 75%, 80%, 85%, 90%, 91%, 92%, 93%, 94%, 95%, 96%, 97%, 98%, 99% of the members of a target organism species. In some embodiments, an antigen binds to an antibody and/or T cell receptor, and may or may not induce a particular physiological response in an organism. In some embodiments, for example, an antigen may bind to an antibody and/or to a T cell receptor in vitro, whether or not such an interaction occurs in vivo. In some embodiments, an antigen reacts with the products of specific humoral or cellular immunity, including those induced by heterologous immunogens.

Associated with: Two events or entities are “associated” with one another, as that term is used herein, if the presence, level and/or form of one is correlated with that of the other. For example, a particular entity (e.g., polypeptide) is considered to be associated with a particular disease, disorder, or condition, if its presence, level and/or form correlates with incidence of and/or susceptibility of the disease, disorder, or condition (e.g., across a relevant population). In some embodiments, two or more entities are physically “associated” with one another if they interact, directly or indirectly, so that they are and remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof. In some embodiments, the term “associated with”, as in reference to an “antigen associated with a cancer cell” refers to the presence of a particular antigen on surface of a cancer cell.

Binding: It will be understood that the term “binding”, as used herein, typically refers to a non-covalent association between or among two or more entities. “Direct” binding involves physical contact between entities or moieties; indirect binding involves physical interaction by way of physical contact with one or more intermediate entities. Binding between two or more entities can be assessed in any of a variety of contexts—including where interacting entities or moieties are studied in isolation or in the context of more complex systems (e.g., while covalently or otherwise associated with a carrier entity and/or in a biological system or cell). As used herein, “Kₐ” refers to an association rate of a particular binding moiety and a target to form a binding moiety/target complex. As used herein, “K_{d}” refers to a dissociation rate of a particular binding moiety/target complex. As used herein, “K_{D}” refers to a dissociation constant, which is obtained from the ratio of K_{d} to Kₐ (i.e., K_{d}/Kₐ) and is expressed as a molar concentration (M). K_{D} values can be determined using methods well established in the art, e.g., by using surface plasmon resonance, or using a biosensor system such as a Biacore® system.

Carrier: As used herein, the term “carrier” refers to a diluent, adjuvant, excipient, or vehicle with which a composition is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components.

Characteristic Portion: As used herein, the term “characteristic portion” is used, in the broadest sense, to refer to a portion of a substance whose presence (or absence) correlates with presence (or absence) of a particular feature, attribute, or activity of the substance. In some embodiments, a characteristic portion of a substance is a portion that is found in the substance and in related substances that share the particular feature, attribute or activity, but not in those that do not share the particular feature, attribute or activity.

Chimeric Antigen Receptor: The term “chimeric antigen receptor” or “CAR” as used herein refers to an engineered receptor which consists of one or more of an extracellular target binding domain (e.g., derived from an antibody), a transmembrane region, and one or more intracellular effector domains. CARs are usually introduced into immune cells, such as T cells, to redirect specificity for a desired cell-surface antigen or MHC-peptide complex. These synthetic receptors typically contain a target binding domain that is associated with one or more signaling domains via a flexible linker in a single fusion molecule. The target binding domain is used to direct the immune cell (e.g., a T cell) to specific targets on the surface of pathologic cells (e.g., a cancer cell) and the signaling domains contain molecular machinery for immune cell (e.g., T cell) activation and proliferation. The flexible linker which usually passes through the immune cell (e.g., T cell) membrane (i.e., forming a transmembrane domain) allows for cell membrane display of the target binding domain of the CAR. CARs have successfully allowed immune cells (e.g., T cells) to be redirected against antigens expressed at the surface of tumor cells from various malignancies including lymphomas and solid tumors (Gross et al., (1989) Transplant Proc., 21(1 Pt 1): 127-30; Jena et al., (2010) Blood, 116(7):1035-44). A CAR's extracellular binding domain may be composed of a single chain variable fragment (scFv) derived from fusing the variable heavy and light regions of a murine or humanized monoclonal antibody. In some embodiments, the extracellular binding domain comprises a single domain antibody. Alternatively, scFvs may be used that are derived from Fab's (instead of from an antibody, e.g., obtained from Fab libraries). In various embodiments, this scFv is fused to a transmembrane domain and then to an intracellular signaling domain.

At least three generations of CARs have been developed. The first generation CARs comprised target binding domains attached to a signaling domain derived from the cytoplasmic region of the CD3zeta or the Fc receptor gamma chains. First generation CARs were shown to successfully redirect T cells to the selected target, but they failed to provide prolonged expansion and antitumor activity in vivo. Second and third generation CARs have focused on enhancing modified T cell survival and increasing proliferation by including co-stimulatory molecules, such as CD28, OX-40 (CD134) and 4-1BB (CD137). The embodiments described herein focus, in part, on further improving CAR-T containing immunotherapies, e.g., by armoring a CAR-T with a TGFβ signaling pathway modulator, thereby making the immunotherapies more effective when treating cancers, especially solid tumor cancers. Armored CARs provided herein improve or enhance the CAR-T function and survival in the face of a hostile tumor microenvironment relative to an unarmored CAR-T cell.

Codon-optimized: As used herein, a “codon-optimized” nucleic acid sequence refers to a nucleic acid sequence that has been altered such that translation of the nucleic acid sequence and expression of the resulting protein is improved optimized for a particular expression system. A “codon-optimized” nucleic acid sequence encodes the same protein as a non-optimized parental sequence upon which the “codon-optimized” nucleic acid sequence is based. For example, a nucleic acid sequence may be “codon-optimized” for expression in mammalian cells (e.g., CHO cells, human cells, mouse cells etc.), bacterial cells (e.g., E. coli), insect cells, yeast cells or plant cells.

Comparable: The term “comparable”, as used herein, refers to two or more agents, entities, situations, sets of conditions, etc. that may not be identical to one another but that are sufficiently similar to permit comparison there between so that conclusions may reasonably be drawn based on differences or similarities observed. Those of ordinary skill in the art will understand, in context, what degree of identity is required in any given circumstance for two or more such agents, entities, situations, sets of conditions, etc. to be considered comparable.

Corresponding to: As used herein, the term “corresponding to” is often used to designate the position/identity of an amino acid residue in a polypeptide of interest. Those of ordinary skill will appreciate that, for purposes of simplicity, residues in a polypeptide are often designated using a canonical numbering system based on a reference related polypeptide, so that an amino acid “corresponding to” a residue at position 190, for example, need not actually be the 190th amino acid in a particular amino acid chain but rather corresponds to the residue found at 190 in the reference polypeptide; those of ordinary skill in the art readily appreciate how to identify “corresponding” amino acids.

Derived from: As used herein the phrase, a sequence “derived from” or “specific for a designated sequence” refers to a sequence that comprises a contiguous sequence of approximately at least 6 nucleotides or at least 2 amino acids, at least about 9 nucleotides or at least 3 amino acids, at least about 10-12 nucleotides or 4 amino acids, or at least about 15-21 nucleotides or 5-7 amino acids corresponding, i.e., identical or complementary to, e.g., a contiguous region of the designated sequence. In certain embodiments, the sequence comprises all of a designated nucleotide or amino acid sequence. The sequence may be complementary (in the case of a polynucleotide sequence) or identical to a sequence region that is unique to a particular sequence as determined by techniques known in the art. Regions from which sequences may be derived, include but are not limited to, regions encoding specific epitopes, regions encoding CDRs, regions encoding framework sequences, regions encoding constant domain regions, regions encoding variable domain regions, as well as non-translated and/or non-transcribed regions. The derived sequence will not necessarily be derived physically from the sequence of interest under study, but may be generated in any manner, including, but not limited to, chemical synthesis, replication, reverse transcription or transcription, that is based on the information provided by the sequence of bases in the region(s) from which the polynucleotide is derived. As such, it may represent either a sense or an antisense orientation of the original polynucleotide. In addition, combinations of regions corresponding to that of the designated sequence may be modified or combined in ways known in the art to be consistent with the intended use. For example, a sequence may comprise two or more contiguous sequences which each comprise part of a designated sequence, and are interrupted with a region which is not identical to the designated sequence but is intended to represent a sequence derived from the designated sequence. With regard to antibody molecules, “derived therefrom” includes an antibody molecule which is functionally or structurally related to a comparison antibody, e.g., “derived therefrom” includes an antibody molecule having similar or substantially the same sequence or structure, e.g., having the same or similar CDRs, framework or variable regions. “Derived therefrom” for an antibody also includes residues, e.g., one or more, e.g., 2, 3, 4, 5, 6 or more residues, which may or may not be contiguous, but are defined or identified according to a numbering scheme or homology to general antibody structure or three-dimensional proximity, i.e., within a CDR or a framework region, of a comparison sequence. The term “derived therefrom” is not limited to physically derived therefrom but includes generation by any manner, e.g., by use of sequence information from a comparison antibody to design another antibody.

Determine: Many methodologies described herein include a step of “determining”. Those of ordinary skill in the art, reading the present specification, will appreciate that such “determining” can utilize any of a variety of techniques available to those skilled in the art, including for example specific techniques explicitly referred to herein. In some embodiments, a determination involves manipulation of a physical sample. In some embodiments, a determination involves consideration and/or manipulation of data or information, for example utilizing a computer or other processing unit adapted to perform a relevant analysis. In some embodiments, a determination involves receiving relevant information and/or materials from a source. In some embodiments, determining involves comparing one or more features of a sample or entity to a comparable reference.

Engineered: The term “engineered”, as used herein, describes a polynucleotide, polypeptide or a cell that has been designed or modified by man and/or whose existence and production require human intervention and/or activity. For example, an engineered cell that is intentionally designed to elicit a particular effect and that differs from the effect of naturally occurring cells of the same type. In some embodiments, an engineered cell expresses a chimeric antigen receptor described herein.

Effector functions: As used herein, the term “effector functions” refers to those biological activities attributable to an antigen binding agent described herein. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptors; and B cell activation. “Reduced or minimized” antibody effector function means that which is reduced by at least 50% (alternatively 60%, 65%, 70%, 75%, 80%, 85%, 90%, 95%, 96%, 97%, 98%, or 99%) from the wild type or unmodified antibody. The determination of antibody effector function is readily determinable and measurable by one of ordinary skill in the art. In some embodiments, the antibody effector functions of complement binding, complement dependent cytotoxicity and antibody dependent cytotoxicity are affected. In some embodiments, effector function is eliminated through a mutation in the constant region that eliminated glycosylation, e.g., “effector-less mutation.” In one aspect, the effector-less mutation is an N297A or DANA mutation (D265A+N297A) in the CH2 region. Shields et al., J. Biol. Chem. 276(9): 6591-6604 (2001). Alternatively, additional mutations resulting in reduced or eliminated effector function include: K322A and L234A/L235A (LALA). Alternatively, effector function can be reduced or eliminated through production techniques, such as expression in host cells that do not glycosylate (e.g., E. coli) or in which result in an altered glycosylation pattern that is ineffective or less effective at promoting effector function (e.g., Shinkawa et al., J. Biol. Chem. 278 (5):3466-3473 (2003).

Epitope: As used herein, the term “epitope” includes any moiety that is specifically recognized by an immunoglobulin (e.g., antibody or receptor) binding component in whole or in part. In some embodiments, an epitope is comprised of a plurality of amino acids in an antigen. In some embodiments, such amino acid residues are surface-exposed when the antigen adopts a relevant three-dimensional conformation. In some embodiments, the amino acid residues are physically near to or contour with each other in space when the antigen adopts such a conformation. In some embodiments, at least some of the amino acids are physically separated from one another when the antigen adopts an alternative conformation (e.g., is linearized; e.g., a non-linear epitope).

Excipient: As used herein, the term “excipient” refers to a non-therapeutic agent that may be included in a pharmaceutical composition, for example to provide or contribute to a desired consistency or stabilizing effect. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Expression: The term “expression” or “expressed”, when used in reference to a nucleic acid herein, refers to one or more of the following events: (1) production of an RNA transcript of a DNA template (e.g., by transcription); (2) processing of an RNA transcript (e.g., by splicing, editing, 5′ cap formation, and/or 3′ end formation); (3) translation of an RNA into a polypeptide; and/or (4) post-translational modification of a polypeptide.

Ex vivo: As used herein, the term “ex vivo” means a process in which cells are removed from a living organism and are propagated outside the organism (e.g., in a test tube, in a culture bag, in a bioreactor).

Fusion protein: As used herein, the term “fusion protein” refers to a protein encoded by a nucleic acid sequence engineered from nucleic acid sequences encoding at least a portion of two different (e.g., heterologous) proteins. As persons of skill are no doubt aware, to create a fusion protein nucleic acid sequences are joined such that the resulting reading does not contain an internal stop codon.

Host: The term “host” is used herein to refer to human or any non-human animal (e.g., mouse, rat, rabbit, dog, cat, cattle, swine, sheep, horse, non-human primate) or a system (e.g., a cell or cell line). In some embodiments, a host is an organism who is being administered a cell or population of cells expressing a CAR and/or a TGFβ modulator described herein. In some embodiments, administering the population of cells results in an improved immune response in the host.

Host cell: As used herein, the phrase “host cell” refers to a cell into which exogenous DNA (recombinant or otherwise) has been introduced. For example, host cells may be used to produce a modified CAR molecule as described herein by standard recombinant techniques. Persons of skill upon reading this disclosure will understand that such terms refer not only to the particular subject cell, but, to the progeny of such a cell. Because certain modifications may occur in succeeding generations due to either mutation or environmental influences, such progeny may not, in fact, be identical to the parent cell, but are still included within the scope of the term “host cell” as used herein.

In some embodiments, host cells refer to human cells. In some embodiments, host cells include any prokaryotic and eukaryotic cells suitable for expressing an exogenous DNA (e.g., a recombinant nucleic acid sequence). Exemplary cells include those of prokaryotes and eukaryotes (single-cell or multiple-cell), bacterial cells (e.g., strains of E. coli, Bacillus spp., Streptomyces spp., etc.), mycobacteria cells, fungal cells, yeast cells (e.g., S. cerevisiae, S. pombe, P. pastoris, P. methanolica, etc.), plant cells, insect cells (e.g., SF-9, SF-21, baculovirus-infected insect cells, Trichoplusia ni, etc.), non-human animal cells, human cells, or cell fusions such as, for example, hybridomas or quadromas. In some embodiments, the cell is a human, monkey, ape, hamster, rat, or mouse cell. In some embodiments, the cell is eukaryotic and is selected from the following cells: CHO (e.g., CHO KI, DXB-11 CHO, Veggie-CHO), COS (e.g., COS-7), retinal cell, Vero, CV1, kidney (e.g., HEK293, HEK293T, 293 EBNA, MSR 293, MDCK, HaK, BHK), HeLa, HepG2, W138, MRC 5, Colo205, HB 8065, HL-60, (e.g., BHK21), Jurkat, Daudi, A431 (epidermal), CV-1, U937, 3T3, L cell, C127 cell, SP2/0, NS-0, MMT 060562, Sertoli cell, BRL 3A cell, HT1080 cell, myeloma cell, tumor cell, and a cell line derived from an aforementioned cell. In some embodiments, the cell comprises one or more viral genes, e.g., a retinal cell that expresses a viral gene (e.g., a PER.C6™ cell).

Immune response: As used herein, the term “immune response” refers to a response of a cell of the immune system, such as a B cell, T cell, dendritic cell, macrophage or polymorphonucleocyte, to a stimulus such as an antigen or vaccine. An immune response can include any cell of the body involved in a host defense response, including for example, an epithelial cell that secretes an interferon or a cytokine. An immune response includes, but is not limited to, an innate and/or adaptive immune response. Methods of measuring immune responses are well known in the art and include, for example, measuring proliferation and/or activity of lymphocytes (such as B or T cells), secretion of cytokines or chemokines, inflammation, antibody production and the like. In some embodiments, immune response refers to the immune response observed following administration of armored or unarmored CAR-T cells described herein. In some embodiments, an immune response following administration of armored CAR-T cells described herein is measured by one or more of increase proliferation of the CAR expressing cells, increase IFNg production by CAR expressing cells, increase IL-2 production of CAR expressing cells, increase proliferation of the host immune cells, increase IFNg production by host immune cells

increase IL-2 production of host immune cells, increase antigen presentation by host antigen presenting cells, increase costimulation by host antigen presenting cells, increase activation of the endothelium, or increase tumor homing of immune cells (eg NK cells, T cells, macrophages).

In vitro: As used herein, the term “in vitro” refers to events that occur in an artificial environment, e.g., in a test tube or reaction vessel, in cell culture, etc., rather than within a multi-cellular organism.

In vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Isolated: As used herein, the term “isolated” refers to a substance and/or entity that has been (1) separated from at least some of the components with which it was associated when initially produced (whether in nature and/or in an experimental setting), and/or (2) designed, produced, prepared, and/or manufactured with human intervention. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. To give but one example, in some embodiments, a biological polymer such as a polypeptide or polynucleotide that occurs in nature is considered to be “isolated” when, a) by virtue of its origin or source of derivation is not associated with some or all of the components that accompany it in its native state in nature; b) it is substantially free of other polypeptides or nucleic acids of the same species from the species that produces it in nature; c) is expressed by or is otherwise in association with components from a cell or other expression system that is not of the species that produces it in nature. Thus, for instance, in some embodiments, a polypeptide that is chemically synthesized or is synthesized in a cellular system different from that which produces it in nature is considered to be an “isolated” polypeptide. In some embodiments, a cell may be an “isolated cell” which is separated from the molecular and/or cellular components that naturally accompany the cell. Alternatively or additionally, in some embodiments, a cell that has been subjected to one or more purification techniques may be considered to be an “isolated” cell to the extent that it has been separated from other components a) with which it is associated in nature; and/or b) with which it was associated when initially produced.

Linker: As used herein, the term “linker”, refers to a functional group (e.g., chemical or polypeptide) that covalently attaches two or more polypeptides or nucleic acids so that they are connected to one another. As used herein, a “peptide linker” refers to one or more amino acids used to couple two proteins together (e.g., to couple VH and VL domains).

Modulate or Modulator: As used herein, the term “modulate” or “modulator” refers to the ability of a component to positively or negatively alter an associated function. Exemplary modulations include a about 1%, about 2%, about 5%, about 10%, about 25%, about 50%, about 75%, or about 100% change. For example, provided herein are TGFB signaling modulators capable of altering or preventing TGFβ receptor from signaling. A person skilled in the art would understand that this can be achieved by either binding the cytokine (i.e., TGFβ) which activates the signaling of TGFβR, or the receptor itself (e.g., a TGFβ antibody or fragment thereof, a TGFBR antibody or fragment thereof). Therefore this term encompasses both molecules which bind TGFβ and molecules which bind TGFβR. In one embodiment, the modulator of the disclosure can neutralize TGFβ signaling through TGFβRII. By “neutralizing”, it is meant that the normal signaling effect of TGFβ is blocked such that the presence of TGFβ has a neutral effect on TGFβRII signaling. In some embodiments, TGFβ modulators improve the immune response in a host.

Nucleic acid: As used herein, the phrase “nucleic acid”, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to individual nucleic acid residues (e.g., nucleotides and/or nucleosides); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxyguanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is single stranded; in some embodiments, a nucleic acid is double stranded. In some embodiments a nucleic acid has a nucleotide sequence comprising at least one element that encodes, or is the complement of a sequence that encodes, a polypeptide. In some embodiments, a nucleic acid has enzymatic activity.

Pharmaceutically acceptable vehicles: The pharmaceutically acceptable carriers (vehicles) useful in this disclosure are conventional. Remington's Pharmaceutical Sciences, by E. W. Martin, Mack Publishing Co., Easton, PA, 15^{th} Edition (1975), describes compositions and formulations suitable for pharmaceutical delivery of one or more therapeutic compositions. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Polypeptide: A “polypeptide”, generally speaking, is a string of at least two amino acids attached to one another by a peptide bond. In some embodiments, a polypeptide may include at least 3-5 amino acids, each of which is attached to others by way of at least one peptide bond. Those of ordinary skill in the art will appreciate that polypeptides sometimes include “non-natural” amino acids or other entities that nonetheless are capable of integrating into a polypeptide chain, optionally. In some embodiments, the term “polypeptide” is used to refer to specific functional classes of polypeptides, such as, an antibody, chimeric antigen receptor, or costimulatory domain polypeptides, etc. For each such class, the present specification provides and/or the art is aware of several examples of amino acid sequences of known exemplary polypeptides within the class; in some embodiments, one or more such known polypeptides is/are reference polypeptides for the class. In such embodiments, the term “polypeptide” refers to any member of the class that shows sufficient sequence homology or identity with a relevant reference polypeptide that one skilled in the art would appreciate that it should be included in the class. In many embodiments, a member of the representative class also shares significant activity with the reference polypeptide. For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (i.e., a conserved region, often including a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids.

It is understood that the antibodies and antigen binding agents of the invention may have additional conservative or non-essential amino acid substitutions, which do not have a substantial effect on the polypeptide functions. Whether or not a particular substitution will be tolerated, i.e., will not adversely affect desired biological properties, such as binding activity, can be determined as described in Bowie, J U et al. Science 247:1306-1310 (1990) or Padlan et al. FASEB J. 9:133-139 (1995). A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Prevention: The term “prevention”, as used herein, refers to prophylaxis, avoidance of disease manifestation, a delay of onset, and/or reduction in frequency and/or severity of one or more symptoms of a particular disease, disorder or condition (e.g., cancer). In some embodiments, prevention is assessed on a population basis such that an agent is considered to “prevent” a particular disease, disorder or condition if a statistically significant decrease in the development, frequency, and/or intensity of one or more symptoms of the disease, disorder or condition is observed in a population susceptible to the disease, disorder, or condition.

Pure: As used herein, an agent or entity is “pure” if it is substantially free of other components. For example, a preparation that contains more than about 90% of a particular agent or entity is typically considered to be a pure preparation. In some embodiments, an agent or entity is at least 91%, at least 92%, at least 93%, at least 94%, at least 95%, at least 96%, at least 97%, at least 98%, or at least 99% pure.

Recombinant: As used herein, the term “recombinant” is intended to refer to polypeptides (e.g., polypeptides as described herein) that are designed, engineered, prepared, expressed, created or isolated by recombinant means, such as polypeptides expressed using a recombinant expression vector transfected into a host cell, polypeptides isolated from a recombinant, combinatorial polypeptide library or polypeptides prepared, expressed, created or isolated by any other means that involves splicing selected sequence elements to one another. In some embodiments, one or more of such selected sequence elements is found in nature. In some embodiments, one or more of such selected sequence elements and/or combinations thereof is designed in silico. In some embodiments, one or more such selected sequence elements results from the combination of multiple (e.g., two or more) known sequence elements that are not naturally present in the same polypeptide.

Reference: The term “reference” is often used herein to describe a standard or control agent, individual, population, sample, sequence or value against which an agent, individual, population, sample, sequence or value of interest is compared. In some embodiments, a reference agent, individual, population, sample, sequence or value is tested and/or determined substantially simultaneously with the testing or determination of the agent, individual, population, sample, sequence or value of interest. In some embodiments, a reference agent, individual, population, sample, sequence or value is a historical reference, optionally embodied in a tangible medium. Typically, as would be understood by those skilled in the art, a reference agent, individual, population, sample, sequence or value is determined or characterized under conditions comparable to those utilized to determine or characterize the agent, individual, population, sample, sequence or value of interest.

Single domain antibody: as used herein, the terms “single domain antibody (sdAb)”, “variable single domain” or “immunoglobulin single variable domain (ISV)” “single heavy chain variable domain (V_{H}) antibody” refer to the single variable fragment of an antibody that binds to a target antigen. These terms are used interchangeably herein. A sdAb is a single antigen-binding polypeptide having three complementary determining regions (CDRs). The sdAb alone is capable of binding to the antigen without pairing with a corresponding CDR-containing polypeptide. In some cases, single-domain antibodies are engineered from camelid HCAbs, and their heavy chain variable domains are referred to as “VHHs”. Some VHHs may also be known as Nanobodies. Camelid sdAb is one of the smallest known antigen-binding antibody fragments (see, e.g., Hamers-Casterman et al., Nature 363: 446-8 (1993); Greenberg et al., Nature 374: 168-73 (1995); Hassanzadeh-Ghassabeh et al., Nanomedicine (Lond), 8:1013-26 (2013)). A basic VHH has the following structure from the N-terminus to the C-terminus: FR1-CDR1-FR2-CDR2-FR3-CDR3-FR4, in which FR1 to FR4 refer to framework regions 1 to 4, respectively, and in which CDR1 to CDR3 refer to the complementarity determining regions 1 to 3. Camelid VHH domains may be humanised according to standard techniques available in the art, and such domains are considered to be “domain antibodies”. As used herein V_{H} includes camelid VHH domains and they term VHH may be used to refer to domain antibodies of human or camelid origin comprising only a heavy chain. As explained below, some embodiments of the various aspects of the invention relate to a binding agent comprising a single heavy chain variable domain antibodies/immunoglobulin heavy chain single variable domain which bind a TGFB antigen in the absence of light chain.

Subject: As used herein, the term “subject” means any mammal, including humans. In certain embodiments of the present invention the subject is an adult, an adolescent or an infant. In some embodiments, terms “individual” or “patient” are used and are intended to be interchangeable with “subject”. Also contemplated by the present invention are the administration of the pharmaceutical compositions and/or performance of the methods of treatment in-utero. For example, a subject can be a patient (e.g., a human patient or a veterinary patient), having a cancer, (e.g., of gastrointestinal origin), a symptom of a cancer, in which at least some of the cells express TGFβ, or a predisposition toward a cancer, in which at least some of the cells express TGFβ. The term “non-human animals” of the invention includes all non-human vertebrates, e.g., non-human mammals and non-mammals, such as non-human primates, sheep, dog, cow, chickens, amphibians, reptiles, etc., unless otherwise noted.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Therapeutic agent: As used herein, the term “therapeutic agent” refers to an agent (e.g., an antigen binding agent) that has biological activity. The term is used herein to denote a chemical compound, a mixture of chemical compounds, a biological macromolecule, or an extract made from biological materials. In some embodiments, the therapeutic agent may be an anti-cancer agent or a chemotherapeutic agent. As used herein, the terms “anti-cancer agent” or “chemotherapeutic agent” refer to agents that have the functional property of inhibiting a development or progression of a neoplasm in a human, particularly a malignant (cancerous) lesion, such as a carcinoma, sarcoma, lymphoma, or leukemia. Inhibition of metastasis or angiogenesis is frequently a property of anti-cancer or chemotherapeutic agents. A chemotherapeutic agent may be a cytotoxic or cytostatic agent. The term “cytostatic agent” refers to an agent which inhibits or suppresses cell growth and/or multiplication of cells.

Transformation: As used herein, refers to any process by which exogenous DNA is introduced into a host cell. Transformation may occur under natural or artificial conditions using various methods well known in the art. Transformation may rely on any known method for the insertion of foreign nucleic acid sequences into a prokaryotic or eukaryotic host cell. In some embodiments, a particular transformation methodology is selected based on the host cell being transformed and may include, but is not limited to, viral infection, electroporation, mating, lipofection. In some embodiments, a “transformed” cell is stably transformed in that the inserted DNA is capable of replication either as an autonomously replicating plasmid or as part of the host chromosome. In some embodiments, a transformed cell transiently expresses introduced nucleic acid for limited periods of time.

Transforming Growth Factor-β (TGFβ): As used herein, the terms “TGF-beta”, “TGFb”, “TGFβ” and “transforming growth factor-beta” are used interchangeably herein and refer to the family of molecules that have either the full-length, native amino acid sequence of any of the TGF-betas from humans, including the latent forms and associated or unassociated complex of precursor and mature TGF-beta (“latent TGF-beta”). Reference to such TGF-beta herein will be understood to be a reference to any one of the currently identified forms, including TGF-beta1, TGF-beta2, TGF-beta3, TGF-beta4, and TGF-beta5 and latent versions thereof, as well as to human TGF-beta species identified in the future, including polypeptides derived from the sequence of any known TGF-beta and being at least about 75%, preferably at least about 80%, more preferably at least about 85%, still more preferably at least about 90%, and even more preferably at least about 95% homologous with the sequence. The specific terms “TGF-beta1,” “TGF-beta2,” and “TGF-beta3”, as well as “TGF-beta4” and “TGF-beta5,” refer to the TGF-betas defined in the literature, e.g., Derynck et al., Nature, supra, Seyedin et al., J. Biol. Chem., 262, supra, and deMartin et al., supra. The term “TGF-beta” refers to the gene encoding human TGF-beta. Preferred TGF-beta is native-sequence human TGF-beta.

Members of the TGF-beta family are defined as those that have nine cysteine residues in the mature portion of the molecule, share at least 65% homology with other known TGF-beta sequences in the mature region, and may compete for the same receptor. In addition, they all appear to be encoded as a larger precursor that shares a region of high homology near the N-terminus and shows conservation of three cysteine residues in the portion of the precursor that will later be removed by processing. Moreover, the TGF-betas appear to have a processing site with four or five amino acids.

Transforming Growth Factor-β Receptor (TGFβR): As used herein, the term “TGF-bR” or “TGF-b receptor” or “TGF-beta receptor” or “TGFβR” is used to encompass all three sub-types of the TGFβR family (i.e., TGFβR1, TGFβR2, TGFβR3). The TGFβ receptors are characterized by serine/threonine kinase activity and exist in several different isoforms that can be homo- or heterodimeric.

TGFβ signaling pathway modulator or TGFβ modulator: As used herein, the term “TGFβ signaling pathway modulator” or “TGFβ modulator”, as used interchangeably herein, refers to a molecule (e.g., an antibody or fragment thereof) which is capable of modulating TGFβ signaling pathway (e.g., having an inhibiting, blocking or neutralizing effect), which may either bind TGFβ itself or it may bind a TGFβ receptor on cells. In either case, the modulator inhibits the TGFβ signaling pathway (e.g., by either binding the cytokine (i.e., TGFβ) itself) or by binding the receptor for TGFβ. Therefore this term encompasses both types of modulators, which bind TGFβ and those which bind the TGFβ receptor. In various embodiments described herein a TGFβ signaling pathway modulator is expressed along with a chimeric antigen receptor in a modified immune cell (e.g., a CAR-T cell). CAR-T cells expressing such a TGFβ signaling pathway modulator are referred to herein as TGFβ armored CAR-T cells.

Treat or treatment: As used herein, the term “treat” or “treatment” is defined as the administration of therapeutic agent to a subject, e.g., a patient, or administration, e.g., by application, to an isolated tissue or cell from a subject which is returned to the subject. In some embodiments, the therapeutic agent is an armored CAR-T cell (e.g., an engineered CAR T-cell that co-expresses a TGFβ modulator). The treatment can be to cure, heal, alleviate, relieve, alter, remedy, ameliorate, palliate, improve or affect the disorder, the symptoms of the disorder or the predisposition toward the disorder, e.g., a cancer. While not wishing to be bound by theory, treating is believed to cause the inhibition, ablation, or killing of a cell in vitro or in vivo, or otherwise reducing capacity of a cell, e.g., an aberrant cell, to mediate a disorder, e.g., a disorder as described herein (e.g., a cancer).

The invention described herein is used in an “effective amount” for therapeutic, prophylactic or preventative treatment. A therapeutically effective amount of the armored CAR-T cells (e.g., engineered cells that co-express a CAR and a TGFβ signaling modulator) described herein is an amount effective to ameliorate or reduce one or more symptoms of, or to prevent or cure, the disease (e.g., cancer).

Variable region or domain: As used herein, the terms “variable region” or “variable domain” of an antibody refers to the amino-terminal domains of the heavy or light chain of an antibody. The variable domains of the heavy chain and light chain may be referred to as “VH” and “VL”, respectively. These domains are generally the most variable parts of the antibody (relative to other antibodies of the same class) and contain the antigen binding sites. Heavy-chain only antibodies have a single heavy chain variable region.

Vector: As used herein, the term “vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors”.

### DETAILED DESCRIPTION OF CERTAIN EMBODIMENTS

The present invention provides for methods and compositions for enhancing the immune response toward cancers and pathogens using a modified immune cell (e.g., a CAR-T cell) armored with a polypeptide that modulates TGFβ signaling. The present invention is not limited to particular methods, and experimental conditions described, as such methods and conditions may vary. It is also to be understood that the terminology used herein is for the purpose of describing particular embodiments only, and is not intended to be limiting unless indicated, since the scope of the present invention will be limited only by the appended claims.

### TGF-B/SMAD Signaling

Transforming growth factor-beta (TGF-β) is a multifunctional cytokine originally named for its ability to transform normal fibroblasts to cells capable of anchorage-independent growth. TGF-β signaling controls many key cellular functions including proliferation, differentiation, survival, migration, and epithelial mesenchymal transition. It regulates diverse biologic processes, such as extracellular matrix formation, wound healing, embryonic development, bone development, hematopoiesis, immune and inflammatory responses, and malignant transformation. Deregulation of TGF-β leads to pathological conditions, e.g., birth defects, cancer, chronic inflammation, and autoimmune and fibrotic diseases.

TGF-β, produced primarily by hematopoietic and tumor cells, can regulate, i.e., stimulate or inhibit, the growth and differentiation of cells from a variety of both normal and neoplastic tissue origins (Spom et al., Science, 233: 532 (1986)) and stimulate the formation and elaboration of various stromal elements. TGF-β is involved in many proliferative and non-proliferative cellular processes such as cell proliferation and differentiation, embryonic development, extracellular matrix formation, bone development, wound healing, hematopoiesis, and immune and inflammatory responses.

TGF-β also possesses immunosuppressive activities, which include lymphokine-activated killer (LAK) and cytotoxic T lymphocyte (CTL) inhibition, depressed B cell lymphopoiesis and kappa light-chain expression, negative regulation of hematopoiesis, down-regulation of HLA-DR expression on tumor cells, and inhibition of the proliferation of antigen-activated B lymphocytes in response to B-cell growth factor. Many human tumors and many tumor cell lines produce TGF-β suggesting a possible mechanism for those tumors to evade normal immunological surveillance. This negative immunomodulation, coupled with the observations that certain transformed cell lines have lost the ability to respond to TGF-β in an autocrine fashion and that TGF-β stimulates stroma formation, and decreases immune surveillance of the tumor, suggests attractive models for neoplasm deregulation and proliferation.

Since TGF-β signaling important for both healthy cells and cancer regulation, targeting TGF-β systemically can cause unwanted side effects. As to cancer specifically, members of the TGF-β family are known to have a number of biological activities related to tumorigenesis (including angiogenesis) and metastasis. TGF-β inhibits the proliferation of many cell types including capillary endothelial cells and smooth muscle cells. TGF-β downregulates integrin expression (alphalbeta1, alpha2beta1, and alphavbeta3 involved in endothelial cell migration). Integrins are involved in the migration of all cells, including metastatic ones. TGF-β downregulates matrix metalloproteinase expression needed for both angiogenesis and metastasis. TGF-β induces plasminogen activator inhibitor, which inhibits a proteinase cascade needed for angiogenesis and metastasis. TGF-β induces normal cells to inhibit transformed cells. See, e.g., Yingling et al., Nature Reviews, 3 (12): 1011-1022 (2004), which discloses that deregulation of TGF-β has been implicated in the pathogenesis of a variety of diseases, including cancer and fibrosis, and presents the rationale for evaluating TGF-β signaling inhibitors as cancer therapeutics, biomarkers/diagnostics, the structures of small-molecule inhibitors that are in development, and the targeted drug discovery model that is being applied to their development.

As used herein, the term “TGF-β signaling pathway” is used to describe the downstream signaling events attributed to TGF-β and TGF-β like ligands. For example, in one signaling pathway a TGF-β ligand binds to and activates a Type II TGF-β receptor. The Type II TGF-β receptor recruits and forms a heterodimer with a Type I TGF-β receptor. The resulting heterodimer permits phosphorylation of the Type I receptor, which in turn phosphorylates and activates a member of the SMAD family of proteins. A signaling cascade is triggered, which is well known to those of skill in the art, and ultimately leads to control of the expression of mediators involved in cell growth, cell differentiation, tumorigenesis, apoptosis, and cellular homeostasis, among others. Other TGF-β signaling pathways are also contemplated for manipulation according to the methods described herein.

### TGF-β Signaling Pathway Modulators

The present invention provides an immune modulating system comprising TGF-β signaling modulators (e.g., polypeptides that modulate TGF-β signaling or nucleic acid sequences encoding polypeptides that modulate TGF-β signaling). In some embodiments, the TGF-β signaling modulators cause a cellular reaction upon binding to TGF-β or TGF-β receptor. In some embodiments, the TGF-β signaling modulators are secreted from a cell.

In various embodiments, the present invention provides modified immune cells (e.g., T cells) that express a chimeric antigen receptor along with a TGF-β signaling modulator. Such a modulator may bind TGF-β itself or a TGF-β receptors. CAR-T cells expressing such modulators are referred to herein as TGF-β armored CAR-T cells.

anti-TGFβ and anti-TGFβR2 Antigen Binding Molecules

In some embodiments, the TGF-β signaling modulators are antigen binding molecules (e.g., antibodies or antigen binding fragments thereof). In some embodiments, the antigen binding molecules (e.g., antibodies or antigen binding fragments thereof) specifically bind to TGF-β. In some embodiments, the antigen binding molecules (e.g., antibodies or antigen binding fragments thereof) specifically bind to TGF-β receptor (TGFβR) (e.g., TGFβR1, TGFβR2).

TGF-β signaling modulators (e.g., an anti-TGFβ antibody molecule or an anti-TGFβR antibody molecule) can comprise all, or an antigen binding subset of the CDRs or the heavy chain, described herein. Exemplary amino acid sequences of anti-TGFβ or anti-TGFβR2 antigen binding agents described herein, including variable regions are shown in Table 1. Additional anti-TGFβ or anti-TGFβR2 antibodies are also described in U.S. Pat. Nos. 7,723,486 and 9,783,604; US Patent application publication Nos. US20160017026A1 and US20180105597, US20190119387; and International Patent Application Nos. WO2012093125A1 WO2011/012609, WO 2017/141208 A1; the entirety of each of which is hereby incorporated by reference. Antigen binding agents useful in the immune modulating system described herein include, but are not limited to, antibodies, bivalent fragments such as (Fab′)2, monovalent fragments such as Fab, single chain antibodies, single chain Fv (scFv), single domain antibodies, multivalent single chain antibodies, diabodies, triabodies, and the like that bind specifically with an antigen (e.g., a TGFβR epitope).

In some embodiments, the immune modulating system comprises a TGF-β signaling modulator (e.g., an anti-TGFβ or anti-TGFβR2 antigen binding agent) that is at least 80%, 85%, 90%, 91%, 92%, 93%, 94%, 95%, 96%, 97%, 98% or 99% identical to a sequence provided in Table 1. In some embodiments, the immune modulating system comprises a TGF-β signaling modulator comprising a one or more CDR sequences of an antibody or fragment thereof described in Table 1. In some embodiments, the a TGF-b signaling modulator of the present invention comprise a heavy chain variable region amino acid sequence that is at least 90%, 91%, 92%, 93%, 94%, 95%, 96%, 97%, 98% or 99% identical to a VH sequence provided in Table 1. In some embodiments, the VH of the TGF-β signaling modulator is a single domain antibody (VH).

In some embodiments, TGF-β signaling modulator comprises a leader sequence. In some embodiments, the TGF-β signaling modulator is a monomer. In some embodiments, the TGF-β signaling modulator is a dimer. In some embodiments, the TGF-β signaling modulator is a trimer.

In some embodiments, the TGF-β signaling modulator comprises a linker to connect domains in tandem. In some embodiments, the linker comprises GGGGS (SEQ ID NO: 59). In some embodiments, the linker comprises (GGGGS)ₙ(SEQ ID NO: 59), where n is 1, 2, 3, 4, 5, 6, 7, 8, 9, or 10. In some embodiments, the linker comprises GGGGSGGGGSGGGGS (SEQ ID NO: 61).

In some embodiments, the TGF-βsignaling modulator comprises a light chain variable region amino acid sequence that is at least 90%, 91%, 92%, 93%, 94%, 95%, 96%, 97%, 98% or 99% identical to a V_{L} sequence provided in Table 1. In some embodiments, the anti-TGFβ antigen binding agents of the present invention comprise a heavy chain variable region amino acid sequence that is identical to a V_{H} sequence provided in Table 1.

In some embodiments, the TGF-βsignaling modulator of the present invention comprise a heavy chain variable region amino acid sequence that is at least 90%, 91%, 92%, 93%, 94%, 95%, 96%, 97%, 98% or 99% identical to a vH sequence provided in Table 1. In some embodiments, the TGF-β signaling modulator of the present invention comprise a heavy chain variable region amino acid sequence that is identical to a vH sequence provided in Table 1.

In some embodiments, the V_{H} of the TGF-βsignaling modulator (e.g., single domain antibody) comprises a leader sequence provided that is at least 90%, 91%, 92%, 93%, 94%, 95%, 96%, 97%, 98% or 99% in Table 1. In some embodiments, the vH anti-TGFβ antigen binding agent (e.g., single domain antibody) comprises a leader sequence provided in Table 1.

TABLE 1
Exemplary anti-TGFβ and anti-TGFβR2 antigen binding molecules

| SEQ ID NO | Sequence |
|---|---|
| SEQ ID NO: 1 | QVQLVQSGAEVKKPGSSVKVSCKASGYTFSSNVISWVRQAPGQ |
| TGFb scFv VH-VL1 | GLEWMGGVIPIVDIANYAQRFKGRVTITADESTSTTYMELSSLRS |
| | EDTAVYYCASTLGLVLDAMDYWGQGTLVTVSSGGGGSGGGGS |
| | GGGGSALETVLTQSPGTLSLSPGERATLSCRASQSLGSSYLAWYQ |
| | QKPGQAPRLLIYGASSRAPGIPDRFSGSGSGTDFTLTISRLEPEDFA |
| | VYYCQQYADSPITFGQGTRLEIKR |
| SEQ ID NO: 2 | QVQLVQSGAEVKKPGSSVKVSCKASGYTFSSNVISWVRQAPGQ |
| TGFb scFv VH-VL2 | GLEWMGGVIPIVDIANYAQRFKGRVTITADESTSTTYMELSSLRS |
| | EDTAVYYCASTLGLVLDAMDYWGQGTLVTVSSGGGGSGGGGS |
| | GGGGSETVLTQSPGTLSLSPGERATLSCRASQSLGSSYLAWYQQ |
| | KPGQAPRLLIYGASSRAPGIPDRFSGSGSGTDFTLTISRLEPEDFAV |
| | YYCQQYADSPITFGQGTRLEIKR |
| SEQ ID NO: 3 | ETVLTQSPGTLSLSPGERATLSCRASQSLGSSYLAWYQQKPGQAP |
| TGFb scFv VL-VH | RLLIYGASSRAPGIPDRFSGSGSGTDFTLTISRLEPEDFAVYYCQQ |
| | YADSPITFGQGTRLEIKRGGGGSGGGGSGGGGSQVQLVQSGAEV |
| | KKPGSSVKVSCKASGYTFSSNVISWVRQAPGQGLEWMGGVIPIV |
| | DIANYAQRFKGRVTITADESTSTTYMELSSLRSEDTAVYYCASTL |
| | GLVLDAMDYWGQGTLVTVSS |
| SEQ ID NO: 4 | QLQVQESGPGLVKPSETLSLTCTVSGGSISNSYFSWGWIRQPPGK |
| TGFbR2 scFv VH-VL | GLEWIGSFYYGEKTYYNPSLKSRATISIDTSKSQFSLKLSSVTAAD |
| | TAVYYCPRGPTMIRGVIDSWGQGTLVTVSSGGGGSGGGGSGGG |
| | GSEIVLTQSPATLSLSPGERATLSCRASQSVRSYLAWYQQKPGQA |
| | PRLLIYDASNRATGIPARFSGSGSGTDFTLTISSLEPEDFAVYYCQ |
| | QRSNWPPTFGQGTKVEIK |
| SEQ ID NO: 5 | EIVLTQSPATLSLSPGERATLSCRASQSVRSYLAWYQQKPGQAPR |
| TGFbR2 scFv VL-VH | LLIYDASNRATGIPARFSGSGSGTDFTLTISSLEPEDFAVYYCQQR |
| | SNWPPTFGQGTKVEIKGGGGSGGGGSGGGGSQLQVQESGPGLV |
| | KPSETLSLTCTVSGGSISNSYFSWGWIRQPPGKGLEWIGSFYYGE |
| | KTYYNPSLKSRATISIDTSKSQFSLKLSSVTAADTAVYYCPRGPT |
| | MIRGVIDSWGQGTLVTVSS |
| SEQ ID NO: 6 | EVQLLESGGGLVQPGGSLRLSCAASGFTFTTYGMGWVRQAPGK |
| mTGFbR2 VH1 | GLEWVSWIEKTGNKTYYADSVKGRFTISRDNSKNTLYLQMNSL |
| | RAEDTAVYYCAKARHIKVRSRDFDYWGQGTLVTVSS |
| SEQ ID NO: 7 | EVQLLESGGGLVQPGGSLRLSCAASGFTFTTYGMGWVRQAPGK |
| | GLEWVSWIEKTGNKTYYADSVKGRFTISRDNSKNTLYLQMNSL |
| | RAEDTAVYYCAKAGRHIKVRSRDFDYWGQGTLVTVSS |
| SEQ ID NO: 8 | EVQLLESGGGLVQPGGSLRLSCAASGFTFGTEQMWWVRQAPGK |
| hTGFbR2 VH1 | GLEFVSRIDSPGGRTYYADSVKGRFTISRD |
| | NSKNTLYLQMNSLRAEDTAVYYCAKRRPTGVSGTFYDYWGQG |
| | TLVTVSS |
| SEQ ID NO: 9 | EVQLLESGGGLVQPGGSLRLSCAASGFTFGTEQMWWVRQAPGK |
| | GLEFVSRIDSPGGRTYYADSVKGRFTISRDNSKNTLYLQMNSLRA |
| | EDTAVYYCAKRRPTGVSGTFYDYWGQGTLVTVSSggggsEVQLLE |
| | SGGGLVQPGGSLRLSCAASGFTFGTEQMWWVRQAPGKGLEFVS |
| | RIDSPGGRTYYADSVKGRFTISRDNSKNTLYLQMNSLRAEDTAV |
| | YYCAKRRPTGVSGTFYDYWGQGTLVTVSS |
| SEQ ID NO: 10 | EVQLLESGGGLVQPGGSLRLSCAASGFTFGTEQMWWVRQAPGK |
| | GLEFVSRIDSPGGRTYYADSVKGRFTISRDNSKNTLYLQMNSLRA |
| | EDTAVYYCAKRRPTGVSGTFYDYWGQGTLVTVSSggggsggggsEV |
| | QLLESGGGLVQPGGSLRLSCAASGFTFGTEQMWWVRQAPGKGL |
| | EFVSRIDSPGGRTYYADSVKGRFTISRDNSKNTLYLQMNSLRAED |
| | TAVYYCAKRRPTGVSGTFYDYWGQGTLVTVSS |
| SEQ ID NO: 11 | EVQLLESGGGLVQPGGSLRLSCAASGFTFGTEQMWWVRQAPGK |
| | GLEFVSRIDSPGGRTYYADSVKGRFTISRDNSKNTLYLQMNSLRA |
| | EDTAVYYCAKRRPTGVSGTFYDYWGQGTLVTVSSGGSEPKSsDK |
| | THTCPPCgggssgggsgGQPREPQVYTLPPSRDELTKNQVSLTCLVKG |
| | FYPSDIAVEWESNGQPENNYKTTPPVLDSDGSFFLYSKLTVDKSR |
| | WQQGNVFSCSVMHEALHNHYTQKSLSLSPG |
| SEQ ID NO: 12 | EVQLLESGGGLVQPGGSLRLSCAASGFTFGTEQMWWVRQAPGK |
| | GLEFVSRIDSPGGRTYYADSVKGRFTISRDNSKNTLYLQMNSLRA |
| | EDTAVYYCAKRRPTGVSGTFYDYWGQGTLVTVSSGGSEPKSsDK |
| | THTCPPCgggssgggsgGQPREPQVYTLPPSRDELTKNQVSLTCLVKG |
| | FYPSDIAVEWESNGQPENNYKTTPPVLDSDGSFFLYSKLTVDKSR |
| | WQQGNVFSCSVMHEALHNHYTQKSLSLSPGggggsggsEVQLLESG |
| | GGLVQPGGSLRLSCAASGFTFGTEQMWWVRQAPGKGLEFVSRI |
| | DSPGGRTYYADSVKGRFTISRDNSKNTLYLQMNSLRAEDTAVYY |
| | CAKRRPTGVSGTFYDYWGQGTLVTVSS |
| SEQ ID NO: 13 | EVQLLESGGGLVQPGGSLRLSCAASGFTFGTEQMWWVRQAPGK |
| | GLEFVSRIDSPGGRTYYADSVKGRFTISRDNSKNTLYLQMNSLRA |
| | EDTAVYYCAKRRPTGVSGTFYDYWGQGTLVTVSSgggssgggsgGQ |
| | PREPQVYTLPPSRDELTKNQVSLTCLVKGFYPSDIAVEWESNGQP |
| | ENNYKTTPPVLDSDGSFFLYSKLTVDKSRWQQGNVFSCSVMHE |
| | ALHNHYTQKSLSLSPG |
| SEQ ID NO: 14 | EVQLLESGGGLVQPGGSLRLSCAASGFTFGTEQMWWVRQAPGK |
| | GLEFVSRIDSPGGRTYYADSVKGRFTISRDNSKNTLYLQMNSLRA |
| | EDTAVYYCAKRRPTGVSGTFYDYWGQGTLVTVSSgggssgggsgGQ |
| | PREPQVYTLPPSRDELTKNQVSLTCLVKGFYPSDIAVEWESNGQP |
| | ENNYKTTPPVLDSDGSFFLYSKLTVDKSRWQQGNVFSCSVMHE |
| | ALHNHYTQKSLSLSPGggggsggsEVQLLESGGGLVQPGGSLRLSCA |
| | ASGFTFGTEQMWWVRQAPGKGLEFVSRIDSPGGRTYYADSVKG |
| | RFTISRDNSKNTLYLQMNSLRAEDTAVYYCAKRRPTGVSGTFYD |
| | YWGQGTLVTVSS |
| SEQ ID NO: 15 | EVQLLESGGGLVQPGGSLRLSCAASGFTFGTEQMWWVRQAPGK |
| | GLEFVSRIDSPGGRTYYADSVKGRFTISRDNSKNTLYLQMNSLRA |
| | EDTAVYYCAKRRPTGVSGTFYDYWGQGTLVTVSSggggsEVQLLE |
| | SGGGLVQPGGSLRLSCAASGFTFGTEQMWWVRQAPGKGLEFVS |
| | RIDSPGGRTYYADSVKGRFTISRDNSKNTLYLQMNSLRAEDTAV |
| | YYCAKRRPTGVSGTFYDYWGQGTLVTVSSggggsEVQLLESGGGL |
| | VQPGGSLRLSCAASGFTFGTEQMWWVRQAPGKGLEFVSRIDSPG |
| | GRTYYADSVKGRFTISRDNSKNTLYLQMNSLRAEDTAVYYCAK |
| | RRPTGVSGTFYDYWGQGTLVTVSS |
| SEQ ID NO: 16 | EVQLLESGGGLVQPGGSLRLSCAASGFTFGTEQMWWVRQAPGK |
| | GLEFVSRIDSPGGRTYYADSVKGRFTISRDNSKNTLYLQMNSLRA |
| | EDTAVYYCAKRRPTGVSGTFYDYWGQGTLVTVSSggggsEVQLLE |
| | SGGGLVQPGGSLRLSCAASGFTFGTEQMWWVRQAPGKGLEFVS |
| | RIDSPGGRTYYADSVKGRFTISRDNSKNTLYLQMNSLRAEDTAV |
| | YYCAKRRPTGVSGTFYDYWGQGTLVTVSSggggsEVQLLESGGGL |
| | VQPGGSLRLSCAASGFTFGTEQMWWVRQAPGKGLEFVSRIDSPG |
| | GRTYYADSVKGRFTISRDNSKNTLYLQMNSLRAEDTAVYYCAK |
| | RRPTGVSGTFYDYWGQGTLVTVSSEVQLLESGGGLVQPGGSLRL |
| | SCAASGFTFGTEQMWWVRQAPGKGLEFVSRIDSPGGRTYYADS |
| | VKGRFTISRDNSKNTLYLQMNSLRAEDTAVYYCAKRRPTGVSGT |
| | FYDYWGQGTLVTVSSggggsggggsEVQLLESGGGLVQPGGSLRLS |
| | CAASGFTFGTEQMWWVRQAPGKGLEFVSRIDSPGGRTYYADSV |
| | KGRFTISRDNSKNTLYLQMNSLRAEDTAVYYCAKRRPTGVSGTF |
| | YDYWGQGTLVTVSSggggsggggsEVQLLESGGGLVQPGGSLRLSC |
| | AASGFTFGTEQMWWVRQAPGKGLEFVSRIDSPGGRTYYADSVK |
| | GRFTISRDNSKNTLYLQMNSLRAEDTAVYYCAKRRPTGVSGTFY |
| | DYWGQGTLVTVSS |
| SEQ ID NO: 17 | QVQLVQSGAEVKKPGSSVKVSCKASGYTFSSNVISWVRQAPGQ |
| | GLEWMGGVIPIVDIANYAQRFKGRVTITADESTSTTYMELSSLRS |
| | EDTAVYYCASTLGLVLDAMDYWGQGTLVTVSSGGGGSGGGGS |
| | GGGGSALETVLTQSPGTLSLSPGERATLSCRASQSLGSSYLAWYQ |
| | QKPGQAPRLLIYGASSRAPGIPDRFSGSGSGTDFTLTISRLEPEDFA |
| | VYYCQQYADSPITFGQGTRLEIKggggsQVQLVQSGAEVKKPGSSV |
| | KVSCKASGYTFSSNVISWVRQAPGQGLEWMGGVIPIVDIANYAQ |
| | RFKGRVTITADESTSTTYMELSSLRSEDTAVYYCASTLGLVLDA |
| | MDYWGQGTLVTVSSGGGGSGGGGSGGGGSALETVLTQSPGTLS |
| | LSPGERATLSCRASQSLGSSYLAWYQQKPGQAPRLLIYGASSRAP |
| | GIPDRFSGSGSGTDFTLTISRLEPEDFAVYYCQQYADSPITFGQGT |
| | RLEIK |
| SEQ ID NO: 18 | QVQLVQSGAEVKKPGSSVKVSCKASGYTFSSNVISWVRQAPGQ |
| | GLEWMGGVIPIVDIANYAQRFKGRVTITADESTSTTYMELSSLRS |
| | EDTAVYYCASTLGLVLDAMDYWGQGTLVTVSSGGGGSGGGGS |
| | GGGGSALETVLTQSPGTLSLSPGERATLSCRASQSLGSSYLAWYQ |
| | QKPGQAPRLLIYGASSRAPGIPDRFSGSGSGTDFTLTISRLEPEDFA |
| | VYYCQQYADSPITFGQGTRLEIKggggsggggsQVQLVQSGAEVKKP |
| | GSSVKVSCKASGYTFSSNVISWVRQAPGQGLEWMGGVIPIVDIA |
| | NYAQRFKGRVTITADESTSTTYMELSSLRSEDTAVYYCASTLGL |
| | VLDAMDYWGQGTLVTVSSGGGGSGGGGSGGGGSALETVLTQSP |
| | GTLSLSPGERATLSCRASQSLGSSYLAWYQQKPGQAPRLLIYGAS |
| | SRAPGIPDRFSGSGSGTDFTLTISRLEPEDFAVYYCQQYADSPITF |
| | GQGTRLEIK |
| SEQ ID NO: 19 | QVQLVQSGAEVKKPGSSVKVSCKASGYTFSSNVISWVRQAPGQ |
| | GLEWMGGVIPIVDIANYAQRFKGRVTITADESTSTTYMELSSLRS |
| | EDTAVYYCASTLGLVLDAMDYWGQGTLVTVSSGGGGSGGGGS |
| | GGGGSALETVLTQSPGTLSLSPGERATLSCRASQSLGSSYLAWYQ |
| | QKPGQAPRLLIYGASSRAPGIPDRFSGSGSGTDFTLTISRLEPEDFA |
| | VYYCQQYADSPITFGQGTRLEIKGGSEPKSSDKTHTCPPCgggssggg |
| | sgGQPREPQVYTLPPSRDELTKNQVSLTCLVKGFYPSDIAVEWES |
| | NGQPENNYKTTPPVLDSDGSFFLYSKLTVDKSRWQQGNVFSCSV |
| | MHEALHNHYTQKSLSLSPG |
| SEQ ID NO: 20 | QVQLVQSGAEVKKPGSSVKVSCKASGYTFSSNVISWVRQAPGQ |
| | GLEWMGGVIPIVDIANYAQRFKGRVTITADESTSTTYMELSSLRS |
| | EDTAVYYCASTLGLVLDAMDYWGQGTLVTVSSGGGGSGGGGS |
| | GGGGSALETVLTQSPGTLSLSPGERATLSCRASQSLGSSYLAWYQ |
| | QKPGQAPRLLIYGASSRAPGIPDRFSGSGSGTDFTLTISRLEPEDFA |
| | VYYCQQYADSPITFGQGTRLEIKGGSEPKSSDKTHTCPPCgggssggg |
| | sgGQPREPQVYTLPPSRDELTKNQVSLTCLVKGFYPSDIAVEWES |
| | NGQPENNYKTTPPVLDSDGSFFLYSKLTVDKSRWQQGNVFSCSV |
| | MHEALHNHYTQKSLSLSPGggggsggsQVQLVQSGAEVKKPGSSVK |
| | VSCKASGYTFSSNVISWVRQAPGQGLEWMGGVIPIVDIANYAQR |
| | FKGRVTITADESTSTTYMELSSLRSEDTAVYYCASTLGLVLDAM |
| | DYWGQGTLVTVSSGGGGSGGGGSGGGGSALETVLTQSPGTLSLS |
| | PGERATLSCRASQSLGSSYLAWYQQKPGQAPRLLIYGASSRAPGI |
| | PDRFSGSGSGTDFTLTISRLEPEDFAVYYCQQYADSPITFGQGTRL |
| | EIK |
| SEQ ID NO: 21 | QVQLVQSGAEVKKPGSSVKVSCKASGYTFSSNVISWVRQAPGQ |
| | GLEWMGGVIPIVDIANYAQRFKGRVTITADESTSTTYMELSSLRS |
| | EDTAVYYCASTLGLVLDAMDYWGQGTLVTVSSGGGGSGGGGS |
| | GGGGSALETVLTQSPGTLSLSPGERATLSCRASQSLGSSYLAWYQ |
| | QKPGQAPRLLIYGASSRAPGIPDRFSGSGSGTDFTLTISRLEPEDFA |
| | VYYCQQYADSPITFGQGTRLEIKgggssgggsgGQPREPQVYTLPPSR |
| | DELTKNQVSLTCLVKGFYPSDIAVEWESNGQPENNYKTTPPVLD |
| | SDGSFFLYSKLTVDKSRWQQGNVFSCSVMHEALHNHYTQKSLS |
| | LSPG |
| SEQ ID NO: 22 | QVQLVQSGAEVKKPGSSVKVSCKASGYTFSSNVISWVRQAPGQ |
| | GLEWMGGVIPIVDIANYAQRFKGRVTITADESTSTTYMELSSLRS |
| | EDTAVYYCASTLGLVLDAMDYWGQGTLVTVSSGGGGSGGGGS |
| | GGGGSALETVLTQSPGTLSLSPGERATLSCRASQSLGSSYLAWYQ |
| | QKPGQAPRLLIYGASSRAPGIPDRFSGSGSGTDFTLTISRLEPEDFA |
| | VYYCQQYADSPITFGQGTRLEIKgggssgggsgGQPREPQVYTLPPSR |
| | DELTKNQVSLTCLVKGFYPSDIAVEWESNGQPENNYKTTPPVLD |
| | SDGSFFLYSKLTVDKSRWQQGNVFSCSVMHEALHNHYTQKSLS |
| | LSPGggggsggsQVQLVQSGAEVKKPGSSVKVSCKASGYTFSSNVIS |
| | WVRQAPGQGLEWMGGVIPIVDIANYAQRFKGRVTITADESTSTT |
| | YMELSSLRSEDTAVYYCASTLGLVLDAMDYWGQGTLVTVSSGG |
| | GGSGGGGSGGGGSALETVLTQSPGTLSLSPGERATLSCRASQSLG |
| | SSYLAWYQQKPGQAPRLLIYGASSRAPGIPDRFSGSGSGTDFTLTI |
| | SRLEPEDFAVYYCQQYADSPITFGQGTRLEIK |
| SEQ ID NO: 23 | QLQVQESGPGLVKPSETLSLTCTVSGGSISNSYFSWGWIRQPPGK |
| | GLEWIGSFYYGEKTYYNPSLKSRATISIDTSKSQFSLKLSSVTAAD |
| | TAVYYCPRGPTMIRGVIDSWGQGTLVTVSSGGGGSGGGGSGGG |
| | GSEIVLTQSPATLSLSPGERATLSCRASQSVRSYLAWYQQKPGQA |
| | PRLLIYDASNRATGIPARFSGSGSGTDFTLTISSLEPEDFAVYYCQ |
| | QRSNWPPTFGQGTKVEIKggggsQLQVQESGPGLVKPSETLSLTCT |
| | VSGGSISNSYFSWGWIRQPPGKGLEWIGSFYYGEKTYYNPSLKSR |
| | ATISIDTSKSQFSLKLSSVTAADTAVYYCPRGPTMIRGVIDSWGQ |
| | GTLVTVSSGGGGSGGGGSGGGGSEIVLTQSPATLSLSPGERATLS |
| | CRASQSVRSYLAWYQQKPGQAPRLLIYDASNRATGIPARFSGSG |
| | SGTDFTLTISSLEPEDFAVYYCQQRSNWPPTFGQGTKVEIK |
| SEQ ID NO: 24 | QLQVQESGPGLVKPSETLSLTCTVSGGSISNSYFSWGWIRQPPGK |
| | GLEWIGSFYYGEKTYYNPSLKSRATISIDTSKSQFSLKLSSVTAAD |
| | TAVYYCPRGPTMIRGVIDSWGQGTLVTVSSGGGGSGGGGSGGG |
| | GSEIVLTQSPATLSLSPGERATLSCRASQSVRSYLAWYQQKPGQA |
| | PRLLIYDASNRATGIPARFSGSGSGTDFTLTISSLEPEDFAVYYCQ |
| | QRSNWPPTFGQGTKVEIKggggsggggsQLQVQESGPGLVKPSETLS |
| | LTCTVSGGSISNSYFSWGWIRQPPGKGLEWIGSFYYGEKTYYNPS |
| | LKSRATISIDTSKSQFSLKLSSVTAADTAVYYCPRGPTMIRGVIDS |
| | WGQGTLVTVSSGGGGSGGGGSGGGGSEIVLTQSPATLSLSPGER |
| | ATLSCRASQSVRSYLAWYQQKPGQAPRLLIYDASNRATGIPARFS |
| | GSGSGTDFTLTISSLEPEDFAVYYCQQRSNWPPTFGQGTKVEIK |
| SEQ ID NO: 25 | QLQVQESGPGLVKPSETLSLTCTVSGGSISNSYFSWGWIRQPPGK |
| | GLEWIGSFYYGEKTYYNPSLKSRATISIDTSKSQFSLKLSSVTAAD |
| | TAVYYCPRGPTMIRGVIDSWGQGTLVTVSSGGGGSGGGGSGGG |
| | GSEIVLTQSPATLSLSPGERATLSCRASQSVRSYLAWYQQKPGQA |
| | PRLLIYDASNRATGIPARFSGSGSGTDFTLTISSLEPEDFAVYYCQ |
| | QRSNWPPTFGQGTKVEIKGGSEPKSSDKTHTCPPCgggssgggsgGQP |
| | REPQVYTLPPSRDELTKNQVSLTCLVKGFYPSDIAVEWESNGQPE |
| | NNYKTTPPVLDSDGSFFLYSKLTVDKSRWQQGNVFSCSVMHEA |
| | LHNHYTQKSLSLSPG |
| SEQ ID NO: 26 | QLQVQESGPGLVKPSETLSLTCTVSGGSISNSYFSWGWIRQPPGK |
| | GLEWIGSFYYGEKTYYNPSLKSRATISIDTSKSQFSLKLSSVTAAD |
| | TAVYYCPRGPTMIRGVIDSWGQGTLVTVSSGGGGSGGGGSGGG |
| | GSEIVLTQSPATLSLSPGERATLSCRASQSVRSYLAWYQQKPGQA |
| | PRLLIYDASNRATGIPARFSGSGSGTDFTLTISSLEPEDFAVYYCQ |
| | QRSNWPPTFGQGTKVEIKGGSEPKSSDKTHTCPPCgggssgggsgGQP |
| | REPQVYTLPPSRDELTKNQVSLTCLVKGFYPSDIAVEWESNGQPE |
| | NNYKTTPPVLDSDGSFFLYSKLTVDKSRWQQGNVFSCSVMHEA |
| | LHNHYTQKSLSLSPGggggsggsQLQVQESGPGLVKPSETLSLTCTV |
| | SGGSISNSYFSWGWIRQPPGKGLEWIGSFYYGEKTYYNPSLKSRA |
| | TISIDTSKSQFSLKLSSVTAADTAVYYCPRGPTMIRGVIDSWGQG |
| | TLVTVSSGGGGSGGGGSGGGGSEIVLTQSPATLSLSPGERATLSC |
| | RASQSVRSYLAWYQQKPGQAPRLLIYDASNRATGIPARFSGSGS |
| | GTDFTLTISSLEPEDFAVYYCQQRSNWPPTFGQGTKVEIK |
| SEQ ID NO: 27 | QLQVQESGPGLVKPSETLSLTCTVSGGSISNSYFSWGWIRQPPGK |
| | GLEWIGSFYYGEKTYYNPSLKSRATISIDTSKSQFSLKLSSVTAAD |
| | TAVYYCPRGPTMIRGVIDSWGQGTLVTVSSGGGGSGGGGSGGG |
| | GSEIVLTQSPATLSLSPGERATLSCRASQSVRSYLAWYQQKPGQA |
| | PRLLIYDASNRATGIPARFSGSGSGTDFTLTISSLEPEDFAVYYCQ |
| | QRSNWPPTFGQGTKVEIKgggssgggsgGQPREPQVYTLPPSRDELTK |
| | NQVSLTCLVKGFYPSDIAVEWESNGQPENNYKTTPPVLDSDGSFF |
| | LYSKLTVDKSRWQQGNVFSCSVMHEALHNHYTQKSLSLSPG |
| SEQ ID NO: 28 | QLQVQESGPGLVKPSETLSLTCTVSGGSISNSYFSWGWIRQPPGK |
| | GLEWIGSFYYGEKTYYNPSLKSRATISIDTSKSQFSLKLSSVTAAD |
| | TAVYYCPRGPTMIRGVIDSWGQGTLVTVSSGGGGSGGGGSGGG |
| | GSEIVLTQSPATLSLSPGERATLSCRASQSVRSYLAWYQQKPGQA |
| | PRLLIYDASNRATGIPARFSGSGSGTDFTLTISSLEPEDFAVYYCQ |
| | QRSNWPPTFGQGTKVEIKgggssgggsgGQPREPQVYTLPPSRDELTK |
| | NQVSLTCLVKGFYPSDIAVEWESNGQPENNYKTTPPVLDSDGSFF |
| | LYSKLTVDKSRWQQGNVFSCSVMHEALHNHYTQKSLSLSPGggg |
| | gsggsQLQVQESGPGLVKPSETLSLTCTVSGGSISNSYFSWGWIRQP |
| | PGKGLEWIGSFYYGEKTYYNPSLKSRATISIDTSKSQFSLKLSSVT |
| | AADTAVYYCPRGPTMIRGVIDSWGQGTLVTVSSGGGGSGGGGS |
| | GGGGSEIVLTQSPATLSLSPGERATLSCRASQSVRSYLAWYQQKP |
| | GQAPRLLIYDASNRATGIPARFSGSGSGTDFTLTISSLEPEDFAVY |
| | YCQQRSNWPPTFGQGTKVEIK |
| SEQ ID NO: 29 | EVQLLESGGGLVQPGGSLRLSCAASGFTFGQESMYWVRQAPGK |
| | GLEWVSAISGSGGSTYYADSVKGRFTISRDNSKNTLYLQMNSLR |
| | AEDTAVYYCAKSGTRIKQGFDYWGQGTLVTVSS |
| SEQ ID NO: 30 | EVQLLESGGGLVQPGGSLRLSCAASGSTFTEYRMWWVRQAPGK |
| | GLEWVSAIEPIGHRTYYANSVRGRFTISRDNSKNTLYLQMNSLRA |
| | EDTAVYYCAKQAPGEKWARRWDLDYWGQGTLVTVSS |
| SEQ ID NO: 31 | EVQLLESGGGLVQPGGSLRLSCAASGFTFGTDQMWWVRQAPGK |
| | GLEFVSRIDSPGGRTYYANSVKGRFTISRDNSKNTLYLQMNSLRA |
| | EDTAVYYCAKRQPAGVSGKYVDYWGQGTLVTVSS |

In some embodiments, the anti-TGFβ or anti-TGFβR antigen binding agent is an antibody. The naturally occurring mammalian antibody structural unit is typified by a tetramer. Each tetramer is composed of two pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains can be classified as kappa and lambda light chains. Heavy chains can be classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)). The variable regions of each light/heavy chain pair form the antibody binding site. Preferred isotypes for the anti-TGFβ antibody molecules are IgG immunoglobulins, which can be classified into four subclasses, IgG1, IgG2, IgG3 and IgG4, having different gamma heavy chains. Most therapeutic antibodies are human, chimeric, or humanized antibodies of the IgG1 type. In a particular embodiment, the anti-TGFβ antibody molecule has the IgG1 isotype.

The variable regions of each heavy and light chain pair form the antigen binding site. Thus, an intact IgG antibody has two binding sites which are the same. However, bifunctional or bispecific antibodies are artificial hybrid constructs which have two different heavy/light chain pairs, resulting in two different binding sites.

The chains all exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987); Chothia et al. Nature 342:878-883 (1989). As used herein, CDRs are referred to for each of the heavy (HCDR1, HCDR2, HCDR3) and light (LCDR1, LCDR2, LCDR3) chains.

Thus, in an embodiment the antibody molecule includes one or both of:
(a) one, two, three, or an antigen binding number of, light chain CDRs (LCDR1, LCDR2 and/or LCDR3) of one of the above-referenced human hybridoma, selected lymphocyte, or murine antibodies. In embodiments the CDR(s) may comprise an amino acid sequence of one or more or all of LCDR1-3 as follows: LCDR1, or modified LCDR1 wherein one to seven amino acids are conservatively substituted) LCDR2, or modified LCDR2 wherein one or two amino acids are conservatively substituted); or LCDR3, or modified LCDR3 wherein one or two amino acids are conservatively substituted; and
(b) one, two, three, or an antigen binding number of, heavy chain CDRs (HCDR1, HCDR2 and/or HCDR3) of one of the above-referenced human hybridoma, selected lymphocyte, or murine antibodies. In embodiments the CDR(s) may comprise an amino acid sequence of one or more or all of HCDR1-3 as follows: HCDR1, or modified HCDR1 wherein one or two amino acids are conservatively substituted; HCDR2, or modified HCDR2 wherein one to four amino acids are conservatively substituted; or HCDR3, or modified HCDR3 wherein one or two amino acids are conservatively substituted.

In some embodiments, an anti-TGFβ antibody molecule or an anti-TGFβR (e.g., an anti-TGFβR2) antibody molecule of the invention can draw antibody-dependent cellular cytotoxicity (ADCC) to a cell expressing TGFβ, e.g., a tumor cell. Antibodies with the IgG1 and IgG3 isotypes are useful for eliciting effector function in an antibody-dependent cytotoxic capacity, due to their ability to bind the Fc receptor. Antibodies with the IgG2 and IgG4 isotypes are useful to minimize an ADCC response because of their low ability to bind the Fc receptor. In related embodiments substitutions in the Fc region or changes in the glycosylation composition of an antibody, e.g., by growth in a modified eukaryotic cell line, can be made to enhance the ability of Fc receptors to recognize, bind, and/or mediate cytotoxicity of cells to which anti-TGFβ antibodies or anti-TGFβR (e.g., an anti-TGFβR2) antibodies bind (see, e.g., U.S. Pat. Nos. 7,317,091, 5,624,821 and publications including WO 00/42072, Shields, et al. J Biol. Chem. 276:6591-6604 (2001), Lazar et al. Proc. Natl. Acad. Sci. U.S.A. 103:4005-4010 (2006), Satoh et al. Expert Opin Biol. Ther. 6:1161-1173 (2006)). In certain embodiments, the antibody or antigen-binding fragment (e.g., antibody of human origin, human antibody) can include amino acid substitutions or replacements that alter or tailor function (e.g., effector function). For example, a constant region of human origin (e.g., γ1 constant region, γ2 constant region) can be designed to reduce complement activation and/or Fc receptor binding. (See, for example, U.S. Pat. No. 5,648,260 (Winter et al.), U.S. Pat. No. 5,624,821 (Winter et al.) and U.S. Pat. No. 5,834,597 (Tso et al.), the entire teachings of which are incorporated herein by reference.) Preferably, the amino acid sequence of a constant region of human origin that contains such amino acid substitutions or replacements is at least about 95% identical over the full length to the amino acid sequence of the unaltered constant region of human origin, more preferably at least about 99% identical over the full length to the amino acid sequence of the unaltered constant region of human origin. Additional anti-TGFβ antigen binding molecules are further described in U.S. Pat. No. 8,785,600 (Nam et al.), the entire teachings of which are incorporated herein by reference.

In still another embodiment, effector functions can also be altered by modulating the glycosylation pattern of the antibody. By altering is meant deleting one or more carbohydrate moieties found in the antibody, and/or adding one or more glycosylation sites that are not present in the antibody. For example, antibodies with enhanced ADCC activities with a mature carbohydrate structure that lacks fucose attached to an Fc region of the antibody are described in U.S. Patent Application Publication No. 2003/0157108 (Presta). See also U.S. Patent Application Publication No. 2004/0093621 (Kyowa Hakko Kogyo Co., Ltd). Glycofi has also developed yeast cell lines capable of producing specific glycoforms of antibodies.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which are engineered to express recombinant antibodies of the invention to thereby produce an antibody with altered glycosylation. For example, EP 1,176,195 by Hang et al. describes a cell line with a functionally disrupted FUT8 gene, which encodes a fucosyl transferase, such that antibodies expressed in such a cell line exhibit hypofucosylation. PCT Publication WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields, R. L. et al., 2002 J Biol. Chem. 277:26733-26740). PCT Publication WO 99/54342 by Umana et al. describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures which results in increased ADCC activity of the antibodies (see also Umana et al., 1999 Nat. Biotech. 17:176-180).

Humanized antibodies can also be made using a CDR-grafted approach. Techniques of generation of such humanized antibodies are known in the art. Generally, humanized antibodies are produced by obtaining nucleic acid sequences that encode the variable heavy and variable light sequences of an antibody that binds to TGFβ, identifying the complementary determining region or “CDR” in the variable heavy and variable light sequences and grafting the CDR nucleic acid sequences on to human framework nucleic acid sequences. (See, for example, U.S. Pat. Nos. 4,816,567 and 5,225,539). The location of the CDRs and framework residues can be determined (see, Kabat, E. A., et al. (1991) Sequences of Proteins of Immunological Interest, Fifth Edition, U.S. Department of Health and Human Services, NIH Publication No. 91-3242, and Chothia, C. et al. J. Mol. Biol. 196:901-917 (1987)).

In some embodiments, the immune modulating system of the present invention comprises a nucleic acid sequence encoding an anti-TGFβ or anti-TGFβR antibody molecule comprising a CDR from an antibody molecule described in Table 1. In some embodiments sequences from Tables 1 can be incorporated into molecules which recognize TGFβ or TGFβR for use in the therapeutic methods described herein (e.g., the immune modulating system, immunoresponsive cells, or methods of treatment comprising the same). The human framework that is selected is one that is suitable for in vivo administration, meaning that it does not exhibit immunogenicity. For example, such a determination can be made by prior experience with in vivo usage of such antibodies and studies of amino acid similarities. A suitable framework region can be selected from an antibody of human origin having at least about 65% amino acid sequence identity, and preferably at least about 70%, 80%, 90% or 95% amino acid sequence identity over the length of the framework region within the amino acid sequence of the equivalent portion (e.g., framework region) of the donor antibody, e.g., an anti-TGFβ antibody molecule. Amino acid sequence identity can be determined using a suitable amino acid sequence alignment algorithm, such as CLUSTAL W, using the default parameters. (Thompson J. D. et al., Nucleic Acids Res. 22:4673-4680 (1994).)

Once the CDRs and FRs of the cloned antibody that are to be humanized are identified, the amino acid sequences encoding the CDRs are identified and the corresponding nucleic acid sequences grafted on to selected human FRs. This can be done using known primers and linkers, the selection of which are known in the art. All of the CDRs of a particular human antibody may be replaced with at least a portion of a non-human CDR or only some of the CDRs may be replaced with non-human CDRs. It is only necessary to replace the number of CDRs required for binding of the humanized antibody to a predetermined antigen. After the CDRs are grafted onto selected human FRs, the resulting “humanized” variable heavy and variable light sequences are expressed to produce a humanized Fv or humanized antibody that binds to TGFβ or TGFβR. Preferably, the CDR-grafted (e.g., humanized) antibody binds TGFβ or TGFβR with an affinity similar to, substantially the same as, or better than that of the donor antibody. Typically, the humanized variable heavy and light sequences are expressed as a fusion protein with human constant domain sequences so an intact antibody that binds to TGFβ is obtained. However, a humanized Fv antibody can be produced that does not contain the constant sequences.

Also within the scope of the invention are humanized antibodies in which specific amino acids have been substituted, deleted or added. In particular, humanized antibodies can have amino acid substitutions in the framework region, such as to improve binding to the antigen. For example, a selected, small number of acceptor framework residues of the humanized immunoglobulin chain can be replaced by the corresponding donor amino acids. Locations of the substitutions include amino acid residues adjacent to the CDR, or which are capable of interacting with a CDR (see e.g., U.S. Pat. Nos. 5,585,089 or 5,859,205). The acceptor framework can be a mature human antibody framework sequence or a consensus sequence. As used herein, the term “consensus sequence” refers to the sequence found most frequently, or devised from the most common residues at each position in a sequence in a region among related family members. A number of human antibody consensus sequences are available, including consensus sequences for the different subgroups of human variable regions (see, Kabat, E. A., et al., Sequences of Proteins of Immunological Interest, Fifth Edition, U.S. Department of Health and Human Services, U.S. Government Printing Office (1991)). The Kabat database and its applications are freely available on line, e.g. via IgBLAST at the National Center for Biotechnology Information, Bethesda, Md. (also see, Johnson, G. and Wu, T. T., Nucleic Acids Research 29:205-206 (2001)).

In certain embodiments, the TGFβ or TGFβR antibody molecule is a human anti-TGFβ or anti-TGFβR IgG1 antibody. Since such antibodies possess desired binding to the TGFβ or TGFβR molecule, any one of such antibodies can be readily isotype-switched to generate a human IgG4 isotype, for example, while still possessing the same variable region (which defines the antibody's specificity and affinity, to a certain extent). Accordingly, as antibody candidates are generated that meet desired “structural” attributes as discussed above, they can generally be provided with at least certain additional “functional” attributes that are desired through isotype switching.

### Single Chain Antibodies

Single chain antibodies lack some or all of the constant domains of the whole antibodies from which they are derived. Therefore, they can overcome some of the problems associated with the use of whole antibodies. For example, single-chain antibodies tend to be free of certain undesired interactions between heavy-chain constant regions and other biological molecules. Additionally, single-chain antibodies are considerably smaller than whole antibodies and can have greater permeability than whole antibodies, allowing single-chain antibodies to localize and bind to target antigen-binding sites more efficiently. Furthermore, the relatively small size of single-chain antibodies makes them less likely to provoke an unwanted immune response in a recipient than whole antibodies.

In some embodiments, the TGFβ signaling modulators are single chain antigen binding molecules (e.g., scFv) that specifically bind to TGFβ. In some embodiments, the TGFβ signaling modulators are single chain antigen binding molecules (e.g., scFv) that specifically bind to TGF-B receptor (TGFβR) (e.g., TGFβR1, TGFβR2).

Multiple single chain antibodies, each single chain having one VH and one VL domain covalently linked by a first peptide linker, can be covalently linked by at least one or more peptide linker to form multivalent single chain antibodies, which can be monospecific or multispecific. Each chain of a multivalent single chain antibody includes a variable light chain fragment and a variable heavy chain fragment, and is linked by a peptide linker to at least one other chain. The peptide linker is composed of at least fifteen amino acid residues. The maximum number of linker amino acid residues is approximately one hundred.

Two single chain antibodies can be combined to form a diabody, also known as a bivalent dimer. Diabodies have two chains and two binding sites, and can be monospecific or bispecific. Each chain of the diabody includes a VH domain connected to a VL domain. The domains are connected with linkers that are short enough to prevent pairing between domains on the same chain, thus driving the pairing between complementary domains on different chains to recreate the two antigen-binding sites.

Three single chain antibodies can be combined to form triabodies, also known as trivalent trimers. Triabodies are constructed with the amino acid terminus of a VL or VH domain directly fused to the carboxyl terminus of a VL or VH domain, i.e., without any linker sequence. The triabody has three Fv heads with the polypeptides arranged in a cyclic, head-to-tail fashion. A possible conformation of the triabody is planar with the three binding sites located in a plane at an angle of 120 degrees from one another. Triabodies can be monospecific, bispecific or trispecific.

### Single-Domain Antibodies

Single-domain antibodies (sdAbs) are different from conventional 4-chain antibodies by having a single monomeric antibody variable domain. For example, camelids and sharks produce sdAbs named heavy chain-only antibodies (HcAbs), which naturally lack light chains. The antigen-binding fragment in each arm of the camelid heavy-chain only antibodies has a single heavy chain variable domain (VHH), which can have high affinity to an antigen without the aid of a light chain. Camelid VHH is known as the smallest functional antigen-binding fragment with a molecular weight of approximately 15 kD.

One aspect of the present application provides isolated single-domain antibodies (referred herein as “anti-TGFβR sdAbs”) that specifically bind to TGFβR, such as human TGFβR2. In some embodiments, the anti-TGFβR sdAb modulates TGFβ activity. In some embodiments, the anti-TGFβ sdAb is an antagonist antibody. Further provided are antigen-binding fragments derived from any one of the anti-TGFβR sdAbs described herein, and antigen binding proteins comprising any one of the anti-TGFβR sdAbs described herein. In some embodiments, the anti-TGFβR sdAb comprise one, two and/or three CDR sequences provided in Table 1. Exemplary anti-TGFβR sdAbs are provided in Table 1.

In some embodiments, some or all of the CDRs sequences, the heavy chain, can be used in another antigen binding agent, e.g., in a CDR-grafted, humanized, or chimeric antibody molecule. Embodiments include an antibody molecule that comprises sufficient CDRs, e.g., all three CDRs from one of the above-referenced heavy chain variable region, to allow binding to TGFβ.

In some embodiments, the CDRs, e.g., all of the HCDRs, are embedded in human or human derived framework region(s). Examples of human framework regions include human germline framework sequences, human germline sequences that have been affinity matured (either in vivo or in vitro), or synthetic human sequences, e.g., consensus sequences. In an embodiment the heavy chain framework is an IgG1 or IgG2 framework.

In some embodiments, the TGFβ modulators of the present invention comprise a heavy chain variable region amino acid sequence provided in Table 1. In some embodiments, the anti-TGFβ antigen binding agents are single domain heavy chain only antibodies (e.g., antigen binding agents that do not comprise an immunoglobulin light chain).

Antibody fragments for in vivo therapeutic or diagnostic use can benefit from modifications which improve their serum half-lives. Suitable organic moieties intended to increase the in vivo serum half-life of the antibody can include one, two or more linear or branched moiety selected from a hydrophilic polymeric group (e.g., a linear or a branched polymer (e.g., a polyalkane glycol such as polyethylene glycol, monomethoxy-polyethylene glycol and the like), a carbohydrate (e.g., a dextran, a cellulose, a polysaccharide and the like), a polymer of a hydrophilic amino acid (e.g., polylysine, polyaspartate and the like), a polyalkane oxide and polyvinyl pyrrolidone), a fatty acid group (e.g., a mono-carboxylic acid or a di-carboxylic acid), a fatty acid ester group, a lipid group (e.g., diacylglycerol group, sphingolipid group (e.g., ceramidyl)) or a phospholipid group (e.g., phosphatidyl ethanolamine group). Preferably, the organic moiety is bound to a predetermined site where the organic moiety does not impair the function (e.g., decrease the antigen binding affinity) of the resulting immunoconjugate compared to the non-conjugated antibody moiety. The organic moiety can have a molecular weight of about 500 Da to about 50,000 Da, preferably about 2000, 5000, 10,000 or 20,000 Da. Examples and methods for modifying polypeptides, e.g., antibodies, with organic moieties can be found, for example, in U.S. Pat. Nos. 4,179,337 and 5,612,460, PCT Publication Nos. WO 95/06058 and WO 00/26256, and U.S. Patent Application Publication No. 20030026805.

### TGFβR Extracellular Domain

The TGF-β receptors contemplated for use in the immune modulating system described herein can be any TGF-β receptor including those from the Activin-like kinase family (ALK), the Bone Morphogenic Protein (BMP) family, the Nodal family, the Growth and Differentiation Factors family (GDF), and the TGF-β receptor family of receptors. TGF-β receptors are serine/threonine kinase receptors that effect various growth and differentiation pathways in the cell. In some embodiments, the TGFβ signaling modulator is an engineered recombinant extracellular domain (ECD) of TGFβ receptor (e.g., TGFβR1, TGFβR2). In some embodiments, the TGF-β receptor useful for the immune modulating system described herein is a type II TGF-β receptor (e.g., TGF-βR2).

In some embodiments, the TGFβ modulator comprises a TGFβR provided in Table 2. In some embodiments, the TGFβ modulator comprises a sequence that is at least 80%, at least 90% at least 91%, at least 92%, at least 93%, at least 94%, at least 95%, at least 96%, at least 97%, at least 98%, at least 99% identical to a sequence provided in Table 2.

TABLE 2
Exemplary TGFβR extracellular domains

| | |
|---|---|
| SEQ ID NO: 32 | IPPHVPKSDVEMEAQKDASIHLSCNRTIHPLKHFNSDV |
| mTGFbR2ECD (I24- | MASDNGGAVKLPQLCKFCDVRLSTCDNQKSCMSNCSI |
| K31, F57-L185) | TAICEKPHEVCVAVWRKNDKNITLETVCHDPKLTYHG |
| | FTLEDAASPKCVMKEKKRAGETFFMCACNMEECNDYI |
| | IFSEEYTTSSPDL |
| SEQ ID NO: 33 | LQCFCHLCTKDNFTCVTDGLCFVSVTETTDKVIHNSMC |
| huTGFbR1 + 2 ECD v1 | IAEIDLIPRDRPFVCAPSSKTGSVTTTYCCNQDHCNKIEL |
| | PTTVKSSPGLGPVEGGGGSTIPPHVQKSVNNDMIVTDN |
| | NGAVKFPQLCKFCDVRFSTCDNQKSCMSNCSITSICEK |
| | PQEVCVAVWRKNDENITLETVCHDPKLPYHDFILEDA |
| | ASPKCIMKEKKKPGETFFMCSCSSDECNDNIIFSEEYNT |
| | SNPD |
| | [hTGFbR1-ECD(L34-E125)-hTGFbR2-ECD(A44-D151)] |
| SEQ ID NO: 34 | QCFCHLCTKDNFTCVTDGLCFVSVTETTDKVIHNSMCI |
| huTGFbR1 + 2 ECD v2 | AEIDLIPRDRPFVCAPSSKTGSVTTTYCCNQDHCNGGG |
| | GSAVKFPQLCKFCDVRFSTCDNQKSCMSNCSITSICEKP |
| | QEVCVAVWRKNDENITLETVCHDPKLPYHDFILEDAA |
| | SPKCIMKEKKKPGETFFMCSCSSDECNDNIIFSE |
| | [hTGFbR1-ECD(Q35-N107)-4GS-4GS-hTGFbR2- |
| | ECD(L34-E125)] |
| SEQ ID NO: 35 | LQCFCHLCTKDNFTCETDGLCFVSVTETTDKVIHNSMC |
| mTGFbR1 + 2 ECD vl | IAEIDLIPRDRPFVCAPSSKTGAVTTTYCCNQDHCNKIE |
| | LPTTGPFSEKQSAGLGPVELGGGGSIPPHVPKSVNSDV |
| | MASDNGGAVKLPQLCKFCDVRLSTCDNQKSCMSNCSI |
| | TAICEKPHEVCVAVWRKNDKNITLETVCHDPKLTYHG |
| | FTLEDAASPKCVMKEKKRAGETFFMCACNMEECNDYI |
| | IFSEEYTTSSPD |
| | [mTGFbR1-ECD(L30-L126)-4GS-mTGFbR2-ECD(I24- |
| | K31, F57-D184)] |
| SEQ ID NO: 36 | QCFCHLCTKDNFTCETDGLCFVSVTETTDKVIHNSMCI |
| huTGFbR1 + 2 ECD v2 | AEIDLIPRDRPFVCAPSSKTGAVTTTYCCNQDHCNGGG |
| | GSAVKLPQLCKFCDVRLSTCDNQKSCMSNCSITAICEK |
| | PHEVCVAVWRKNDKNITLETVCHDPKLTYHGFTLEDA |
| | ASPKCVMKE |
| | [mTGFbR1-ECD(Q31-N107)-4GS-mTGFbR2-ECD(I24- |
| | K31, F57-E150)] |
| SEQ ID NO: 37 | METDTLLLWVLLLWVPGSTGIPPHVPKSDVEMEAQKD |
| Mouse TGFbR2 ECD with | ASIHLSCNRTIHPLKHFNSDVMASDNGGAVKLPQLCKF |
| IgKss | CDVRLSTCDNQKSCMSNCSITAICEKPHEVCVAVWRK |
| | NDKNITLETVCHDPKLTYHGFTLEDAASPKCVMKEKK |
| | RAGETFFMCACNMEECNDYIIFSEEYTTSSPDL |
| SEQ ID NO: 38 | METDTLLLWVLLLWVPGSTGIPPHVPKSDVEMEAQKD |
| Mouse TGFbR2 ECD dimer | ASIHLSCNRTIHPLKHFNSDVMASDNGGAVKLPQLCKF |
| with 8(G4S) linker and | CDVRLSTCDNQKSCMSNCSITAICEKPHEVCVAVWRK |
| IgKss with flag tag | NDKNITLETVCHDPKLTYHGFTLEDAASPKCVMKEKK |
| | RAGETFFMCACNMEECNDYIIFSEEYTTSSPDLGGGGS |
| | GGGGSGGGGSGGGGSGGGGSGGGGSGGGGSGGGGSI |
| | PPHVPKSDVEMEAQKDASIHLSCNRTIHPLKHFNSDVM |
| | ASDNGGAVKLPQLCKFCDVRLSTCDNQKSCMSNCSIT |
| | AICEKPHEVCVAVWRKNDKNITLETVCHDPKLTYHGF |
| | TLEDAASPKCVMKEKKRAGETFFMCACNMEECNDYII |
| | FSEEYTTSSPDLGGGGS DYKDDDDK |

### Chimeric Antigen Receptors

In some aspects, the present invention provides an immune modulating system comprising TGF-β signaling modulators (e.g., polypeptides that modulate TGF-β signaling or nucleic acid sequences encoding polypeptides that modulate TGF-β signaling) and a chimeric antigen receptor (CAR) that can bind to an antigen of interest. CARs are hybrid molecules comprising three essential units: (1) an extracellular antigen-binding motif, (2) linking/transmembrane motifs, and (3) intracellular T-cell signaling motifs (Long A H, Haso W M, Orentas R J. Lessons learned from a highly-active CD22-specific chimeric antigen receptor. Oncoimmunology. 2013; 2 (4):e23621 In some embodiments, the CARs of the present invention comprise from the N-terminus to the C-terminus, a signal or leader peptide, an antigen binding domain, a transmembrane and/or hinge domain, a costimulatory domain, and an intracellular domain. In some embodiments, CARs are “first generation CARs”, e.g., include those that solely provide CD3 (signals upon antigen binding, “Second-generation” CARs include those that provide both costimulation (e.g. CD28 or CD137) and activation (CD3). “Third-generation” CARs include those that provide multiple costimulation (e.g. CD28 and CD137) and activation (CD3). In various embodiments, the CAR is selected to have high affinity or avidity for the antigen.

The antigen-binding motif of a CAR is commonly fashioned after a single chain Fragment variable (ScFv), the minimal binding domain of an immunoglobulin (Ig) molecule or single domain antibody (For example, WO2018/028647A1). Alternate antigen-binding motifs, such as receptor ligands (i.e., IL-13 has been engineered to bind tumor expressed IL-13 receptor), intact immune receptors, library-derived peptides, and innate immune system effector molecules (such as NKG2D) also have been engineered. Alternate cell targets for CAR expression (such as NK or gamma-delta T cells) are also under development (Brown C E et al. Clin Cancer Res. 2012; 18(8):2199-209; Lehner M et al. PLoS One. 2012; 7 (2):e31210).

In some embodiments, the antigen binding domain of the CAR is a single chain variable fragment. In some embodiments, the antigen binding domain of the CAR is a single domain antibody. In some embodiments, the CARs comprise from the N-terminus to the C-terminus, a signal or leader peptide, vH, CD28 transmembrane and hinge, CD28 costimulatory domain, and CD3 zeta intracellular domain.

The linking motifs of a CAR can be a relatively stable structural domain, such as the constant domain of IgG, or designed to be an extended flexible linker. Structural motifs, such as those derived from IgG constant domains, can be used to extend the ScFv binding domain away from the T-cell plasma membrane surface. This may be important for some tumor targets where the binding domain is particularly close to the tumor cell surface membrane (such as for the disialoganglioside GD2; Orentas et al., unpublished observations). To date, the signaling motifs used in CARs always include the CD3-ζ chain because this core motif is the key signal for T cell activation. The first reported second-generation CARs featured CD28 signaling domains and the CD28 transmembrane sequence. This motif was used in third-generation CARs containing CD137 (4-1BB) signaling motifs as well (Zhao Y et al. J Immunol. 2009; 183 (9): 5563-74). With the advent of new technology, the activation of T cells with beads linked to anti-CD3 and anti-CD28 antibody, and the presence of the canonical “signal 2” from CD28 was no longer required to be encoded by the CAR itself. Using bead activation, third-generation vectors were found to be not superior to second-generation vectors in in vitro assays, and they provided no clear benefit over second-generation vectors in mouse models of leukemia (Haso W, Lee D W, Shah N N, Stetler-Stevenson M, Yuan C M, Pastan I H, Dimitrov D S, Morgan R A, FitzGerald D J, Barrett D M, Wayne A S, Mackall C L, Orentas R J. Anti-CD22-chimeric antigen receptors targeting B cell precursor acute lymphoblastic leukemia, Blood. 2013; 121 (7):1165-74; Kochenderfer J N et al. Blood. 2012; 119 (12):2709-20). This is borne out by the clinical success of CD19-specific CARs that are in a second generation CD28/CD3-ζ (Lee D W et al. American Society of Hematology Annual Meeting. New Orleans, La.; Dec. 7-10, 2013) and a CD137/CD3-ζ signaling format (Porter D L et al. N Engl J Med. 2011; 365 (8): 725-33). In addition to CD137, other tumor necrosis factor receptor superfamily members such as OX40 also are able to provide important persistence signals in CAR-transduced T cells (Yvon E et al. Clin Cancer Res. 2009; 15(18):5852-60). Equally important are the culture conditions under which the CAR T-cell populations were cultured.

Characteristics of CARs include their ability to redirect T-cell specificity and reactivity toward a selected target in a non-MHC-restricted manner, and exploiting the antigen-binding properties of monoclonal antibodies. The non-MHC-restricted antigen recognition gives T cells expressing CARs the ability to recognize antigen independent of antigen processing, thus bypassing a major mechanism of tumor escape. Moreover, when expressed in T-cells, CARs advantageously do not dimerize with endogenous T cell receptor (TCR) alpha and beta chains.

### Extracellular Domain

As described herein, the CAR comprises a target-specific binding element otherwise referred to as an antigen binding domain or moiety. The choice of domain depends upon the type and number of ligands that define the surface of a target cell. For example, the antigen binding domain may be chosen to recognize a ligand (e.g., a cancer antigen) that acts as a cell surface marker on target cells associated with a particular disease state (e.g., cancer). Thus examples of cell surface markers that may act as ligands for the antigen binding domain in the CAR include those associated with viral, bacterial and parasitic infections, autoimmune disease and cancer cells.

In some embodiments, the extracellular domain of the CAR comprises an antigen binding agent that specifically binds to a cancer antigen. In certain embodiments, the CAR binds to a tumor antigen. Any tumor antigen (antigenic peptide) can be used in the tumor-related embodiments described herein. Sources of antigen include, but are not limited to, cancer proteins. The antigen can be expressed as a peptide or as an intact protein or portion thereof. The intact protein or a portion thereof can be native or mutagenized. Non-limiting examples of tumor antigens include carbonic anhydrase IX (CA1X), carcinoembryonic antigen (CEA), CD8, CD7, CD 10, CD 19, CD20, CD22, CD30, CD33, CLL1, CD34, CD38, CD41, CD44, CD49f, CD56, CD74, CD133, CD138, CD123, CD44V6, an antigen of a cytomegalovirus (CMV) infected cell (e.g., a cell surface antigen), epithelial glycoprotein-2 (EGP-2), epithelial glycoprotein-40 (EGP-40), epithelial cell adhesion molecule (EpCAM), receptor tyrosine-protein kinases erb-B2,3,4 (erb-B2,3,4), folate-binding protein (FBP), fetal acetylcholine receptor (AChR), folate receptor-a, Ganglioside G2 (GD2), Ganglioside G3 (GD3), Guanylyl cyclase C (GCC), human Epidermal Growth Factor Receptor 2 (ITER-2), human tel om erase reverse transcriptase (hTERT), Interleukin-13 receptor subunit alpha-2 (IL-13Rcx2), k-light chain, kinase insert domain receptor (KDR), Lewis Y (LeY), L1 cell adhesion molecule (L1CAM), melanoma antigen family A, 1 (MAGE-A1), Mucin 16 (MUC16), Mucin 1 (MUC1), Mesothelin (MSLN), ERBB2, MAGEA3, p53, MARTl, GP100, Proteinase3 (PRI), Tyrosinase, Survivin, hTERT, EphA2, NKG2D ligands, cancer-testis antigen NY-ES0-1, oncofetal antigen (h5T4), prostate stem cell antigen (PSCA), prostate-specific membrane antigen (PSMA), PTK7 ROR1, tumor-associated glycoprotein 72 (TAG-72), vascular endothelial growth factor R2 (VEGF-R2), and Wilms tumor protein (WT-1), BCMA, NKCS1, EGF1R, EGFR-VIII, CD99, CD70, ADGRE2, CCR1, LILRB2, PRAME CCR4, CD5, CD3, TRBC1, TRBC2, TIM-3, Integrin B7, ICAM-1, CD70, Tim3, CLEC12A and ERBB.

In certain embodiments, the CAR binds to a CD19 polypeptide. In certain embodiments, the CAR binds to a human CD19 polypeptide. In certain embodiments, the CAR binds to the extracellular domain of a CD19 protein. In certain embodiments, the CD19 CAR comprises a sequence provided in Table 3.

In certain embodiments, the CAR binds to a GCC polypeptide. In certain embodiments, the CAR binds to a human GCC polypeptide. In certain embodiments, the CAR binds to the extracellular domain of a GCC protein. In certain embodiments, the anti-GCC CAR comprises a sequence provided in Table 3.

In certain embodiments, the CAR binds to a mesothelin polypeptide. In certain embodiments, the CAR binds to a human mesothelin polypeptide. In certain embodiments, the CAR binds to the extracellular domain of a mesothelin protein.

In certain embodiments, the CAR binds to a pathogen antigen, e.g., for use in treating and/or preventing a pathogen infection or other infectious disease, for example, in an immunocompromised subject. Non-limiting examples of pathogen includes a virus, bacteria, fungi, parasite and protozoa capable of causing disease.

Non-limiting examples of viruses include, Retroviridae (e.g. human immunodeficiency viruses, such as HIV−1 (also referred to as HDTV-III, LAVE or HTLV-IIELAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Naira viruses); Arena viridae (hemorrhagic fever viruses); Reoviridae (e.g. reoviruses, orbiviurses and rotaviruses); Birnaviridae Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=intemally transmitted; class 2=parenterally transmitted (i.e. Hepatitis C); Norwalk and related viruses, and astroviruses).

Non-limiting examples of bacteria include Pasteur ella, Staphylococci, Streptococcus, Escherichia coli, Pseudomonas species, and Salmonella species. Specific examples of infectious bacteria include but are not limited to, Helicobacter pyloris, Borelia burgdorferi, Legionella pneumophilia, Mycobacteria sps (e.g. M. tuberculosis, M. avium, M. intracellular e, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, Corynebacterium diphtheriae, Corynebacterium sp., Erysipelothrix rhusiopathiae, Clostridium perfringers, Clostridium tetani, Enterobacter aerogenes, Klebsiella pneumoniae, Pasteurella multocida, Bacteroides sp., Fusobacterium nucleatum, Streptobacillus moniliformis, Treponema palladium, Treponema pertenue, Leptospira, Rickettsia, and Actinomyces israelii.

In certain embodiments, the pathogen antigen is a viral antigen present in Cytomegalovirus (CMV), a viral antigen present in Epstein Barr Virus (EBV), a viral antigen present in Human Immunodeficiency Virus (HIV), or a viral antigen present in influenza virus.

In certain embodiments, extracellular domain of the CAR comprises a linker. In some embodiments, the linker comprises GGGGS (SEQ ID NO: 59). In some embodiments, the linker comprises (GGGGS)ₙ(SEQ ID NO: 59), where n is 1, 2, 3, 4, 5, 6, 7, 8, 9, or 10. In some embodiments, the linker comprises GGGGSGGGGSGGGGS (SEQ ID NO: 61).

In some embodiments, the extracellular antigen binding domain comprises an IgA antibody, IgG antibody, IgE antibody, IgM antibody, bi- or multi-specific antibody, Fab fragment, Fab′ fragment, F(ab′)2 fragment, Fd′ fragment, Fd fragment, isolated CDRs or sets thereof; single-chain variable fragment (scFv), polypeptide-Fc fusion, single domain antibody (sdAb), camelid antibody; masked antibody, Small Modular ImmunoPharmaceuticals (“SMIPsTM”), single chain, Tandem diabody, VHHs, Anticalin, Nanobody, humabody, minibodies, BiTE, ankyrin repeat protein, DARPIN, Avimer, DART, TCR-like antibody, Adnectin, Affilin, Trans-body; Affibody, TrimerX, MicroProtein, Fynomer, Centyrin; and KALBITOR, or fragments thereof.

In some embodiments, the extracellular antigen binding domain of the CAR comprises a single-chain variable fragment (scFv). In some embodiments, the extracellular antigen binding domain of the CAR comprises single domain antibody (sdAb). In some embodiments, single domain antibody (sdAb),

### Transmembrane Domain

As described herein, the CAR comprises a transmembrane domain. With respect to the transmembrane domain, the CAR comprises one or more transmembrane domains fused to the extracellular antigen binding domain of the CAR. The transmembrane domain may be derived either from a natural or from a synthetic source. Where the source is natural, the domain may be derived from any membrane-bound or transmembrane protein.

Transmembrane regions of particular use in the CARs described herein may be derived from (i.e. comprise at least the transmembrane region(s) of) the alpha, beta or zeta chain of the T-cell receptor, CD28, CD3 epsilon, CD45, CD4, CD5, CD8, CD9, CD16, CD22, mesothelin, CD33, CD37, CD64, CD80, CD86, CD134, CD137, CD154. Alternatively the transmembrane domain may be synthetic, in which case it will comprise predominantly hydrophobic residues such as leucine and valine. In some embodiments, a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. In some embodiments, the linker is a glycine-serine doublet or a triple alanine linker.

In some embodiments, the transmembrane domain that naturally is associated with one of the domains in the CAR is used in addition to the transmembrane domains described supra. In some embodiments, the transmembrane domain can be selected by amino acid substitution to avoid binding of such domains to the transmembrane domains of the same or different surface membrane proteins to minimize interactions with other members of the receptor complex.

In some embodiments, the transmembrane domain in the CAR of the invention is a CD28 transmembrane domain. In some embodiments, the CD28 transmembrane domain comprises the nucleic acid sequence of FWVLVVVGGVLACYSLLVTVAFIIFWV SEQ ID NO: 42. In some embodiments, the CD28 transmembrane domain comprises a nucleic acid sequence that encodes the amino acid sequence of SEQ ID NO: 42. In some embodiments, the transmembrane domain comprises a sequence having at least one, two or three modifications (e.g., substitutions) but not more than 20, 10 or 5 modifications (e.g., substitutions) of an amino acid sequence of SEQ ID NO: 42 or a sequence with at least 95%, 96%, 97%, 98% or 99% identity to an amino acid sequence of SEQ ID NO: 42.

In the CAR, a spacer domain, also termed hinge domain, can be arranged between the extracellular domain and the transmembrane domain, or between the intracellular domain and the transmembrane domain. The spacer domain means any oligopeptide or polypeptide that serves to link the transmembrane domain with the extracellular domain and/or the transmembrane domain with the intracellular domain. The spacer domain comprises up to 300 amino acids, preferably 10 to 100 amino acids, and most preferably 25 to 50 amino acids.

In several embodiments, the linker can include a spacer element, which, when present, increases the size of the linker such that the distance between the effector molecule or the detectable marker and the antibody or antigen binding fragment is increased. Exemplary spacers are known to the person of ordinary skill, and include those listed in U.S. Pat. Nos. 7,964,566 7,498,298, 6,884,869, 6,323,315, 6,239,104, 6,034,065, 5,780,588, 5,665,860, 5,663,149, 5,635,483, 5,599,902, 5,554,725, 5,530,097, 5,521,284, 5,504,191, 5,410,024, 5,138,036, 5,076,973, 4,986,988, 4,978,744, 4,879,278, 4,816,444, and 4,486,414, as well as U.S. Pat. Pub. Nos. 20110212088 and 20110070248, each of which is incorporated by reference herein in its entirety.

The spacer domain preferably has a sequence that promotes binding of a CAR with an antigen and enhances signaling into a cell. Examples of an amino acid that is expected to promote the binding include cysteine, a charged amino acid, and serine and threonine in a potential glycosylation site, and these amino acids can be used as an amino acid constituting the spacer domain.

In some embodiments, the CAR comprises a hinge domain. In some embodiments, the hinge domain comprises the nucleic acid sequence of IEVMYPPPYLDNEKSNGTIIHVKGKHLCPSPLFPGPSKP (SEQ ID NO: 41). In some embodiments, the hinge domain comprises a nucleic acid sequence that encodes the amino acid sequence of SEQ ID NO: 41. In some embodiments, the hinge domain comprises a sequence having at least one, two or three modifications (e.g., substitutions) but not more than 20, 10 or 5 modifications (e.g., substitutions) of an amino acid sequence of SEQ ID NO: 41 or a sequence with 95-99% identity to an amino acid sequence of SEQ ID NO: 41.

In some embodiments, the hinge and transmembrane domains are derived from the same molecule. In other embodiments, the hinge and transmembrane domains are derived from different molecules (e.g., CD8 fused to CD28). In some embodiments, the CAR comprises a hinge domain. In some embodiments, the hinge domain comprises the nucleic acid sequence of IEVMYPPPYLDNEKSNGTIIHVKGKHLCPSPLFPGPSKPFWVLVVVGGVLACYSL LVTVAFIIFWV (SEQ ID NO: 43). In some embodiments, the hinge domain comprises a nucleic acid sequence that encodes the amino acid sequence of SEQ ID NO: 43. In some embodiments, the hinge domain comprises a sequence having at least one, two or three modifications (e.g., substitutions) but not more than 20, 10 or 5 modifications (e.g., substitutions) of an amino acid sequence of SEQ ID NO: 43.

### Intracellular Domain

The cytoplasmic domain or otherwise the intracellular signaling domain of the CAR is responsible for activation of at least one of the normal effector functions of the immune cell in which the CAR has been placed in. The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Examples of intracellular signaling domains for use in the CAR include the cytoplasmic sequences of the T cell receptor (TCR) and co-receptors that act in concert to initiate signal transduction following antigen receptor engagement, as well as any derivative or variant of these sequences and any synthetic sequence that has the same functional capability. Signals generated through the TCR alone are insufficient for full activation of the T cell and that a secondary or co-stimulatory signal is also required. Thus, T cell activation can be said to be mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

Primary cytoplasmic signaling sequences regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences that are of particular use in the CARs disclosed herein include those derived from TCR zeta (CD3 Zeta), FcR gamma, FcR beta, CD3 gamma, CD3 delta, CD3 epsilon, CD5, CD22, CD79a, CD79b, and CD66d. Specific, non-limiting examples, of the ITAM include peptides having sequences of amino acid numbers 51 to 164 of CD3.zeta. (NCBI RefSeq: NP.sub.-932170.1), amino acid numbers 45 to 86 of Fc.epsilon.RI.gamma. (NCBI RefSeq: NP.sub.-004097.1), amino acid numbers 201 to 244 of Fc.epsilon.RI.beta. (NCBI RefSeq: NP.sub.-000130.1), amino acid numbers 139 to 182 of CD3.gamma. (NCBI RefSeq: NP.sub.-000064.1), amino acid numbers 128 to 171 of CD3.delta. (NCBI RefSeq: NP.sub.-000723.1), amino acid numbers 153 to 207 of CD3.epsilon. (NCBI RefSeq: NP.sub.-000724.1), amino acid numbers 402 to 495 of CD5 (NCBI RefSeq: NP.sub.-055022.2), amino acid numbers 707 to 847 of 0022 (NCBI RefSeq: NP.sub.-001762.2), amino acid numbers 166 to 226 of CD79a (NCBI RefSeq: NP.sub.-001774.1), amino acid numbers 182 to 229 of CD79b (NCBI RefSeq: NP.sub.-000617.1), and amino acid numbers 177 to 252 of CD66d (NCBI RefSeq: NP.sub.-001806.2), and their variants having the same function as these peptides have. The amino acid number based on amino acid sequence information of NCBI RefSeq ID or GenBank described herein is numbered based on the full length of the precursor (comprising a signal peptide sequence etc.) of each protein. In one embodiment, the cytoplasmic signaling molecule in the CAR comprises a cytoplasmic signaling sequence derived from CD3 zeta.

In some embodiments, the intracellular domain of the CAR can be designed to comprise the CD3-zeta signaling domain by itself or combined with any other desired cytoplasmic domain(s) useful in the context of the CAR. For example, the intracellular domain of the CAR can comprise a CD3 zeta chain portion and a costimulatory signaling region. The costimulatory signaling region refers to a portion of the CAR comprising the intracellular domain of a costimulatory molecule. A costimulatory molecule is a cell surface molecule other than an antigen receptor or their ligands that is required for an efficient response of lymphocytes to an antigen. Examples of such costimulatory molecules include CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, and the like. Specific, non-limiting examples, of such costimulatory molecules include peptides having sequences of amino acid numbers 236 to 351 of CD2 (NCBI RefSeq: NP.sub.-001758.2), amino acid numbers 421 to 458 of CD4 (NCBI RefSeq: NP.sub.-000607.1), amino acid numbers 402 to 495 of CD5 (NCBI RefSeq: NP.sub.-055022.2), amino acid numbers 207 to 235 of CD8.alpha. (NCBI RefSeq: NP.sub.-001759.3), amino acid numbers 196 to 210 of CD83 (GenBank: AAA35664.1), amino acid numbers 181 to 220 of CD28 (NCBI RefSeq: NP.sub.-006130.1), amino acid numbers 214 to 255 of CD137 (4-1BB, NCBI RefSeq: NP.sub.-001552.2), amino acid numbers 241 to 277 of CD134 (OX40, NCBI RefSeq: NP.sub.-003318.1), and amino acid numbers 166 to 199 of ICOS (NCBI RefSeq: NP.sub.-036224.1), and their variants having the same function as these peptides have. Thus, while the disclosure herein is exemplified primarily with 4-1BB as the co-stimulatory signaling element, other costimulatory elements are within the scope of the disclosure.

The cytoplasmic signaling sequences within the cytoplasmic signaling portion of the CAR may be linked to each other in a random or specified order. Optionally, a short oligo- or polypeptide linker, preferably between 2 and 10 amino acids in length may form the linkage. In some embodiments, the linker is a glycine-serine doublet or a triple alanine linker.

In some embodiments, the intracellular domain is designed to comprise a CD28 costimulatory signaling domain. In some embodiments, the intracellular domain of the CAR comprises an amino acid sequence that is at least 95%, 96%, 97%, 98%, 99% or 100% identical to RSKRSRLLHSDYMNMTPRRPGPTRKHYQPYAPPRDFAAYRS (SEQ ID NO: 44).

In some embodiments, the intracellular domain is designed to comprise the signaling domain of CD3-zeta and the signaling domain of CD28.

In some embodiments, the intracellular domain comprises a CD3-zeta with one or more modified immunoreceptor tyrosine based-activation motifs (ITAMs). In some embodiments, the intracellular domain comprises a CD3-zeta with the first of the three immunoreceptor tyrosine based-activation motifs (ITAMs) unmodified and the second and third ITAMs altered, named “1XX”. In some embodiments, the intracellular domain of the CAR comprises an amino acid sequence that is at least 95%, 96%, 97%, 98%, 99% or 100% identical to

(SEQ ID NO: 45)
RVKFSRSADAPAYQQGQNQLYNELNLGRREEYDVLDKRRGRDPEMGGKP
RRKNPQEGLFNELQKDKMAEAFSEIGMKGERRRGKGHDGLFQGLSTATK
DTFDALHMQALPPR

In some embodiments, the CAR comprises an intracellular signaling domain comprising a modified CD3z polypeptide (e.g., a modified human CD3z polypeptide) comprising a native ITAM1, a native BRS1, a native BRS2, a native BRS3, an ITAM2 variant having two loss-of-function mutations, and an ITAM3 variant having two loss-of-function mutations, and a co-stimulatory signaling region comprising a CD28 polypeptide (e.g., a human CD28 polypeptide).

In another embodiment, the intracellular domain is designed to comprise the signaling domain of CD3-zeta and the signaling domain of 4-1BB. In yet another embodiment, the intracellular domain is designed to comprise the signaling domain of CD3-zeta and the signaling domain of CD28 and 4-1BB.

In some embodiments, the intracellular domain of the CAR is designed to comprise the signaling domain of 4-1BB and the signaling domain of CD3-zeta.

In some embodiments, the CAR comprises an amino acid sequence that is at least 80%, 85%, 90%, 95%, 96%, 97%, 98%, 99% or 100% identical to an amino acid sequences provided in Table 3.

TABLE 3
Exemplary Chimeric Antigen Receptors

| | Sequence |
|---|---|
| Anti-CD19 CAR | MGTSLLCWMALCLLGADHADAQVQLQQSGPELVKPGASVKISCKA |
| | SGYAFSSSWMNWVKQRPGKGLEWIGRIYPGDEDTNYSGKFKDKAT |
| | LTADKSSTTAYMQLSSLTSEDSAVYFCARSLLYGDYLDYWGQGTTL |
| | TVSSGGGGSGGGGSGGGGSQIVLTQSPAIMSASPGEKVTMTCSASSS |
| | VSYMHWYQQKSGTSPKRWIYDTSKLASGVPDRFSGSGSGTSYFLTI |
| | NNMEAEDAATYYCQQWNINPLTFGAGTKLELKRSDPTTTPAPRPPT |
| | PAPTIASQPLSLRPEACRPAAGGAVHTRGLDFACDIFWVLWVGGVL |
| | ACYSLLVTVAFIIFWVRSKRSRLLHSDYMNMTPRRPGPTRKHYQPY |
| | APPRDFAAYRSRDQRLPPDAHKPPGGGSFRTPIQEEQADAHSTLAKI |
| | RVKFSRSADAPAYQQGQNQLYNELNLGRREEYDVLDKRRGRDPEM |
| | GGKPRRKNPQEGLYNELQKDKMAEAYSEIGMKGERRRGKGHDGL |
| | YQGLSTATKDTYDALHMQALPPR (SEQ ID NO: 49) |
| Mouse CD19 CAR | METDTLLLWVLLLWVPGSTGEVKLQQSGAELVRPGSSVKISCKASG |
| | YAFSSYWMNWVKQRPGQGLEWIGQIYPGDGDTNYNGKFKGQATL |
| | TADKSSSTAYMQLSGLTSEDSAVYFCARKTISSVVDFYFDYWGQGT |
| | TVTVSSGGGGSGGGGSGGGGSDIELTQSPKFMSTSVGDRVSVTCKA |
| | SQNVGTNVAWYQQKPGQSPKPLIYSATYRNSGVPDRFTGSGSGTDF |
| | TLTITNVQSKDLADYFCQQYNRYPYTSGGGTKLEIKIEFMYPPPYLD |
| | NERSNGTIIHIKEKHLCHTQSSPKLFWALVVVAGVLFCYGLLVTVAL |
| | CVIWTNSRRNRLLQSDYMNMTPRRPGLTRKPYQPYAPA |
| | RDFAAYRPRAKFSRSAETAANLQDPNQLYNELNLGRREEYDVLEKK |
| | RARDPEMGGKQQRRRNPQEGVYNALQKDKMAEAYSEIGTKGERR |
| | RGKGHDGLYQGLSTATKDTYDALHMQTLAPR (SEQ ID NO: 62) |
| Human CD19 | MALPVTALLLPLALLLHAEVKLQQSGAELVRPGSSVKISCKASGYAF |
| CAR (with CD3- | SSYWMNWVKQRPGQGLEWIGQIYPGDGDTNYNGKFKGQATLTAD |
| 1xx) | KSSSTAYMQLSGLTSEDSAVYFCARKTISSVVDFYFDYWGQGTTVT |
| | VSSGGGGSGGGGSGGGGSDIELTQSPKFMSTSVGDRVSVTCKASQN |
| | VGTNVAWYQQKPGQSPKPLIYSATYRNSGVPDRFTGSGSGTDFTLTI |
| | TNVQSKDLADYFCQQYNRYPYTSGGGTKLEIKRAAAIEVMYPPPYL |
| | DNEKSNGTIIHVKGKHLCPSPLFPGPSKPFWVLVVVGGVLACYSLLV |
| | TVAFIIFWVRSKRSRLLHSDYMNMTPRRPGPTRKHYQPYAPPRDFA |
| | AYRSRVKFSRSADAPAYQQGQNQLYNELNLGRREEYDVLDKRRGR |
| | DPEMGGKPRRKNPQEGLFNELQKDKMAEAFSEIGMKGERRRGKGH |
| | DGLFQGLSTATKDTFDALHMQALPPR (SEQ ID NO: 63) |
| V1-01 anti-GCC | MGWSCIILFLVATATGVHSEVQLQESGPGLVKPSETLSLTCTVSGASI |
| CAR | SHYYWSWFRQPAGKGLEWIGRIYPSGSTSYNPSLKSRVAMSVDTPK |
| | NQFSLKLSSVTAADTAVYYCARDRSTGWSEWNSDLWGRGTLVTVS |
| | SIEVMYPPPYLDNEKSNGTIIHVKGKHLCPSPLFPGPSKPFWVLVVV |
| | GGVLACYSLLVTVAFIIFWVRSKRSRLLHSDYMNMTPRRPGPTRKH |
| | YQPYAPPRDFAAYRSRVKFSRSADAPAYQQGQNQLYNELNLGRRE |
| | EYDVLDKRRGRDPEMGGKPRRKNPQEGLFNELQKDKMAEAFSEIG |
| | MKGERRRGKGHDGLFQGLSTATKDTFDALHMQALPPR (SEQ ID |
| | NO: 47) |
| V51 anti-GCC | MELGLSWVFLVAILEGVQCEVQLVESGGGLVQPGGSLRLSCAASGF |
| CAR | TFSRYWMTWVRQAPGKGLEWVAKIRHDGGEKYYADSVKGRFTISR |
| | DNAKNSLYLQMNSLRAEDTAVYYCTRDYNKDYWGQGTLVTVSSIE |
| | VMYPPPYLDNEKSNGTIIHVKGKHLCPSPLFPGPSKPFWVLVVVGGV |
| | LACYSLLVTVAFIIFWV |
| | RSKRSRLLHSDYMNMTPRRPGPTRKHYQPYAPPRDFAAYRSRVKFS |
| | RSADAPAYQQGQNQLYNELNLGRREEYDVLDKRRGRDPEMGGKP |
| | RRKNPQEGLFNELQKDKMAEAFSEIGMKGERRRGKGHDGLFQGLS |
| | TATKDTFDALHMQALPPR (SEQ ID NO: 48) |
| V5 anti-GCC CAR | MELGLSWVFLVAILEGVQCQVQLVESGGGLVQPGGSLRLSCTASGF |
| | TFSRYWMSWVRQAPGKGLEWVAKIRHDGGEKYYVDSVKGRFTISR |
| | DNAKNSLYLQMNSLRAEDTAVYYCATDYTRDVWGQGTAVTVSSR |
| | AAAIEVMYPPPYLDNEKSNGTIIHVKGKHLCPSPLFPGPSKPFWVLV |
| | VVGGVLACYSLLVTVAFIIFWV |
| | RSKRSRLLHSDYMNMTPRRPGPTRKHYQPYAPPRDFAAYRS |
| | RVKFSRSADAPAYQQGQNQLYNELNLGRREEYDVLDKRRGRDPEM |
| | GGKPRRKNPQEGLFNELQKDKMAEAFSEIGMKGERRRGKGHDGLF |
| | QGLSTATKDTFDALHMQALPPR (SEQ ID NO: 50) |
| V36 anti-GCC | MELGLSWVFLVAILEGVQCEVQLVESGGGLAQPGGSLRLSCAASGF |
| CAR | TFSRYWMTWVRQAPGGRLEWVAKIKYDGSEKYYADSVKGRFTISR |
| | DNAKNSLYLQMDSLRAEDTAVYYCTRDYNKDYWGQGTLVTVSSR |
| | AAAIEVMYPPPYLDNEKSNGTIIHVKGKHLCPSPLFPGPSKPFWVLV |
| | VVGGVLACYSLLVTVAFIIFWV |
| | RSKRSRLLHSDYMNMTPRRPGPTRKHYQPYAPPRDFAAYRS |
| | RVKFSRSADAPAYQQGQNQLYNELNLGRREEYDVLDKRRGRDPEM |
| | GGKPRRKNPQEGLFNELQKDKMAEAFSEIGMKGERRRGKGHDGLF |
| | QGLSTATKDTFDALHMQALPPR (SEQ ID NO: 51) |
| V1 anti-GCC CAR | MGWSCIILFLVATATGVHSQVQLQESGPGLVKPSETLSLTCTVSGASI |
| | SHYYWSWFRQPAGKGLEWIGRIYPSGSTSYNPSLKSRVAMSVDTPK |
| | NQFSLNLSSVTAADTAVYYCARDRSTGWSEWNSDLWGRGTLVTVS |
| | SRAAAIEVMYPPPYLDNEKSNGTIIHVKGKHLCPSPLFPGPSKPFWVL |
| | VVVGGVLACYSLLVTVAFIIFWVRSKRSRLLHSDYMNMTPRRPGPT |
| | RKHYQPYAPPRDFAAYRSRVKFSRSADAPAYQQGQNQLYNELNLG |
| | RREEYDVLDKRRGRDPEMGGKPRRKNPQEGLFNELQKDKMAEAFS |
| | EIGMKGERRRGKGHDGLFQGLSTATKDTFDALHMQALPPR (SEQ ID |
| | NO: 52) |
| V48 anti-GCC | MELGLSWVFLVAILEGVQCEVQLVESGGGLVQPGGSLRLTCAASGF |
| CAR | TFSRYWMTWVRQAPGKGLEWVAKIRHDGGEKYYPDSVKGRFTVS |
| | RDNAKNSLYLQMDNLRAEDTAMYYCTRDYNKDLWGQGTLVTVSS |
| | RAAAIEVMYPPPYLDNEKSNGTIIHVKGKHLCPSPLFPGPSKPFWVL |
| | VVVGGVLACYSLLVTVAFIIFWV |
| | RSKRSRLLHSDYMNMTPRRPGPTRKHYQPYAPPRDFAAYRS |
| | RVKFSRSADAPAYQQGQNQLYNELNLGRREEYDVLDKRRGRDPEM |
| | GGKPRRKNPQEGLFNELQKDKMAEAFSEIGMKGERRRGKGHDGLF |
| | QGLSTATKDTFDALHMQALPPR (SEQ ID NO: 53) |
| V8 anti-GCC CAR | QVQLVESGGGLVQPGGSLRLSCAASGFTFSRYWMSWVRQAPGKGL |
| | EWVAKIKYDGSEKYYVDSVKGRFTISRDNAKNSVYLQMNSLRAED |
| | TGVYYCATDFTRDVWGQGTTVTVSS |
| | RAAAIEVMYPPPYLDNEKSNGTIIHVKGKHLCPSPLFPGPSKPFWVL |
| | VVVGGVLACYSLLVTVAFIIFWV |
| | RSKRSRLLHSDYMNMTPRRPGPTRKHYQPYAPPRDFAAYRS |
| | RVKFSRSADAPAYQQGQNQLYNELNLGRREEYDVLDKRRGRDPEM |
| | GGKPRRKNPQEGLFNELQKDKMAEAFSEIGMKGERRRGKGHDGLF |
| | QGLSTATKDTFDALHMQALPPR (SEQ ID NO: 54) |
| V9 anti-GCC CAR | EVQLVESGGGLVQPGGSLRLSCAASGFTFSRYWMTWVRQAPGRGL |
| | EWVAKIRYDGGEKYYVDSVKGRFTISRDNAKNSLYLQMNSLRAED |
| | TAVYYCATDFTRDVWGQGTTVTVSS |
| | RAAAIEVMYPPPYLDNEKSNGTIIHVKGKHLCPSPLFPGPSKPFWVL |
| | VVVGGVLACYSLLVTVAFIIFWV |
| | RSKRSRLLHSDYMNMTPRRPGPTRKHYQPYAPPRDFAAYRS |
| | RVKFSRSADAPAYQQGQNQLYNELNLGRREEYDVLDKRRGRDPEM |
| | GGKPRRKNPQEGLFNELQKDKMAEAFSEIGMKGERRRGKGHDGLF |
| | QGLSTATKDTFDALHMQALPPR (SEQ ID NO: 55) |
| V30 anti-GCC | QVQLVESGGGLVQPGGSLRLSCAASGFNFGRYWMSWVRQAPGKG |
| CAR | REWVAKIKYDGSEKYYVDSVKGRFTISRDNAKNSLYLQMNSLRAE |
| | DTAVYYCATDFTRDVWGQGTTVTVSS |
| | RAAAIEVMYPPPYLDNEKSNGTIIHVKGKHLCPSPLFPGPSKPFWVL |
| | VVVGGVLACYSLLVTVAFIIFWV |
| | RSKRSRLLHSDYMNMTPRRPGPTRKHYQPYAPPRDFAAYRS |
| | RVKFSRSADAPAYQQGQNQLYNELNLGRREEYDVLDKRRGRDPEM |
| | GGKPRRKNPQEGLFNELQKDKMAEAFSEIGMKGERRRGKGHDGLF |
| | QGLSTATKDTFDALHMQALPPR (SEQ ID NO: 56) |
| V31 anti-GCC | QVQLVESGGGVVRPGGSLRLSCAASGFTFSRYWMSWVRQAPGKGR |
| CAR | EWVAKIKYDGSEKYYADSVKGRFTISRDNAKNSLYLQMNSLRADD |
| | TAVYYCATDFTRDVWGQGTTVTVSS |
| | RAAAIEVMYPPPYLDNEKSNGTIIHVKGKHLCPSPLFPGPSKPFWVL |
| | VVVGGVLACYSLLVTVAFIIFWV |
| | RSKRSRLLHSDYMNMTPRRPGPTRKHYQPYAPPRDFAAYRS |
| | RVKFSRSADAPAYQQGQNQLYNELNLGRREEYDVLDKRRGRDPEM |
| | GGKPRRKNPQEGLFNELQKDKMAEAFSEIGMKGERRRGKGHDGLF |
| | QGLSTATKDTFDALHMQALPPR (SEQ ID NO: 57) |

### Functional Features of CARs

Also expressly included within the scope of the invention are functional portions of the CARs disclosed herein. The term “functional portion” when used in reference to a CAR refers to any part or fragment of one or more of the CARs disclosed herein, which part or fragment retains the biological activity of the CAR of which it is a part (the parent CAR). Functional portions encompass, for example, those parts of a CAR that retain the ability to recognize target cells, or detect, treat, or prevent a disease, to a similar extent, the same extent, or to a higher extent, as the parent CAR. In reference to the parent CAR, the functional portion can comprise, for instance, about 10%, 25%, 30%, 50%, 68%, 80%, 90%, 95%, or more, of the parent CAR.

The functional portion can comprise additional amino acids at the amino or carboxy terminus of the portion, or at both termini, which additional amino acids are not found in the amino acid sequence of the parent CAR. Desirably, the additional amino acids do not interfere with the biological function of the functional portion, e.g., recognize target cells, detect cancer, treat or prevent cancer, etc. More desirably, the additional amino acids enhance the biological activity, as compared to the biological activity of the parent CAR.

Included in the scope of the disclosure are functional variants of the CARs disclosed herein. The term “functional variant” as used herein refers to a CAR, polypeptide, or protein having substantial or significant sequence identity or similarity to a parent CAR, which functional variant retains the biological activity of the CAR of which it is a variant. Functional variants encompass, for example, those variants of the CAR described herein (the parent CAR) that retain the ability to recognize target cells to a similar extent, the same extent, or to a higher extent, as the parent CAR. In reference to the parent CAR, the functional variant can, for instance, be at least about 30%, 50%, 75%, 80%, 90%, 98% or more identical in amino acid sequence to the parent CAR.

A functional variant can, for example, comprise the amino acid sequence of the parent CAR with at least one conservative amino acid substitution. Alternatively or additionally, the functional variants can comprise the amino acid sequence of the parent CAR with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent CAR.

Amino acid substitutions of the CARs are preferably conservative amino acid substitutions. Conservative amino acid substitutions are known in the art, and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, He, Leu, Met, Phe, Pro, Trp, Cys, Val, etc.), a basic/positively charged polar amino acid substituted for another basic/positively charged polar amino acid (e.g. Lys, His, Arg, etc.), an uncharged amino acid with a polar side chain substituted for another uncharged amino acid with a polar side chain (e.g., Asn, Gin, Ser, Thr, Tyr, etc.), an amino acid with a beta-branched side-chain substituted for another amino acid with a beta-branched side-chain (e.g., He, Thr, and Val), an amino acid with an aromatic side-chain substituted for another amino acid with an aromatic side chain (e.g., His, Phe, Trp, and Tyr), etc.

The CAR can consist essentially of the specified amino acid sequence or sequences described herein, such that other components, e.g., other amino acids, do not materially change the biological activity of the functional variant.

The CARs (including functional portions and functional variants) can be of any length, i.e., can comprise any number of amino acids, provided that the CARs (or functional portions or functional variants thereof) retain their biological activity, e.g., the ability to specifically bind to antigen, detect diseased cells in a mammal, or treat or prevent disease in a mammal, etc. For example, the CAR can be about 50 to about 5000 amino acids long, such as 50, 70, 75, 100, 125, 150, 175, 200, 300, 400, 500, 600, 700, 800, 900, 1000 or more amino acids in length.

The CARs (including functional portions and functional variants of the invention) can comprise synthetic amino acids in place of one or more naturally-occurring amino acids. Such synthetic amino acids are known in the art, and include, for example, aminocyclohexane carboxylic acid, norleucine, -amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, a-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, omithine, -aminocyclopentane carboxylic acid, a-aminocyclohexane carboxylic acid, a-aminocycloheptane carboxylic acid, a-(2-amino-2-norbomane)-carboxylic acid, γ-diaminobutyric acid, β-diaminopropionic acid, homophenylalanine, and a-tert-butylglycine.

The CARs (including functional portions and functional variants) can be glycosylated, amidated, carboxylated, phosphorylated, esterified, N-acylated, cyclized via, e.g., a disulfide bridge, or converted into an acid addition salt and/or optionally dimerized or polymerized, or conjugated.

### Substitutions and Variants

In some embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleic acid sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### a) Substitution, Insertion, and Deletion Variants

In some embodiments, antibody variants having one or more amino acid substitutions are provided. Sites of interest for substitutional mutagenesis include the HVRs and FRs. As further described below in reference to amino acid side chain classes. Amino acid substitutions may be introduced into an antibody of interest and the products screened for a desired activity, e.g., retained/improved antigen binding, decreased immunogenicity, or improved ADCC or CDC.

Amino acids may be grouped according to common side-chain properties:
(1) hydrophobic: Norleucine, Met, Ala, Val, Leu, Ile;
(2) neutral hydrophilic: Cys, Ser, Thr, Asn, Gln
(3) acidic: Asp, Glu
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant (s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207: 179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178: 1-37 (O'Brien et al., ed., Human Press, Totowa, NJ, (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In some embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or CDRs. In some embodiments of the variant VHH sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244: 1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

### b) Glycosylation Variants

In some embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15: 26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the present application may be made in order to create antibody variants with certain improved properties.

In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336: 1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249: 533-545 (1986); US Patent Application No. US 2003/0157108 A1, Presta, L; and WO 2004/056312 A1, Adams et al.), and knockout cell lines, such as alpha-1, 6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94 (4):680-688 (2006); and WO2003/085107).

Antibody variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

The CARs (including functional portions and functional variants thereof) can be obtained by methods known in the art. The CARs may be made by any suitable method of making polypeptides or proteins. Suitable methods of de novo synthesizing polypeptides and proteins are described in references, such as Chan et al., Fmoc Solid Phase Peptide Synthesis, Oxford University Press, Oxford, United Kingdom, 2000; Peptide and Protein Drug Analysis, ed. Reid, R., Marcel Dekker, Inc., 2000; Epitope Mapping, ed. Westwood et al., Oxford University Press, Oxford, United Kingdom, 2001; and U.S. Pat. No. 5,449,752. Also, polypeptides and proteins can be recombinantly produced using the nucleic acids described herein using standard recombinant methods. See, for instance, Sambrook et al., Molecular Cloning: A Laboratory Manual, 3rd ed., Cold Spring Harbor Press, Cold Spring Harbor, N.Y. 2001; and Ausubel et al., Current Protocols in Molecular Biology, Greene Publishing Associates and John Wiley & Sons, N Y, 1994. Further, some of the CARs (including functional portions and functional variants thereof) can be isolated and/or purified from a source, such as a plant, a bacterium, an insect, a mammal, e.g., a rat, a human, etc. Methods of isolation and purification are well-known in the art. Alternatively, the CARs described herein (including functional portions and functional variants thereof) can be commercially synthesized by companies. In this respect, the CARs can be synthetic, recombinant, isolated, and/or purified.

### Detectable Markers and Tags

A CAR, a T cell expressing a CAR, monoclonal antibodies, antigen binding fragments thereof, specific for one or more of the antigens disclosed herein, can also expressed with (e.g., co-expressed) with a tag protein. In some embodiments, a furin recognition site and downstream 2A ribosome sequence, designed for simultaneous bicistronic expression of the tag sequence and the CAR sequence. In some embodiments, the 2A sequence comprises the nucleic acid sequence of GSGATNFSLLKQAGDVEENPGP SEQ ID NO: 58. In some embodiments, furin and P2A sequence comprises the nucleic acid sequence that encodes the amino acid sequence of SEQ ID NO: 58. In some embodiments, the P2A tag comprises the amino acid sequence of SEQ ID NO: 58 or a sequence with at least 95%, at least 96%, at least 97%, at least 98%, at least 99% identity thereof.

A CAR, a T cell expressing a CAR, monoclonal antibodies, antigen binding fragments thereof, specific for one or more of the antigens disclosed herein, can also be conjugated with a detectable marker; for example, a detectable marker capable of detection by ELISA, spectrophotometry, flow cytometry, microscopy or diagnostic imaging techniques (such as computed tomography (CT), computed axial tomography (CAT) scans, magnetic resonance imaging (MRI), nuclear magnetic resonance imaging NMRI), magnetic resonance tomography (MTR), ultrasound, fiberoptic examination, and laparoscopic examination). Specific, non-limiting examples of detectable markers include fluorophores, chemiluminescent agents, enzymatic linkages, radioactive isotopes and heavy metals or compounds (for example super paramagnetic iron oxide nanocrystals for detection by MRI). For example, useful detectable markers include fluorescent compounds, including fluorescein, fluorescein isothiocyanate, rhodamine, 5-dimethylamine-1-napthalenesulfonyl chloride, phycoerythrin, lanthanide phosphors and the like. Bioluminescent markers are also of use, such as luciferase, Green fluorescent protein (GFP), Yellow fluorescent protein (YFP). A CAR, a T cell expressing a CAR, an antibody, or antigen binding portion thereof, can also be conjugated with enzymes that are useful for detection, such as horseradish peroxidase, 0-galactosidase, luciferase, alkaline phosphatase, glucose oxidase and the like. When a CAR, a T cell expressing a CAR, an antibody, or antigen binding portion thereof, is conjugated with a detectable enzyme, it can be detected by adding additional reagents that the enzyme uses to produce a reaction product that can be discerned. For example, when the agent horseradish peroxidase is present the addition of hydrogen peroxide and diaminobenzidine leads to a colored reaction product, which is visually detectable. A CAR, a T cell expressing a CAR, an antibody, or antigen binding portion thereof, may also be conjugated with biotin, and detected through indirect measurement of avidin or streptavidin binding. It should be noted that the avidin itself can be conjugated with an enzyme or a fluorescent label.

A CAR, a T cell expressing a CAR, an antibody, or antigen binding portion thereof, may be conjugated with a paramagnetic agent, such as gadolinium. Paramagnetic agents such as superparamagnetic iron oxide are also of use as labels. Antibodies can also be conjugated with lanthanides (such as europium and dysprosium), and manganese. An antibody or antigen binding fragment may also be labeled with a predetermined polypeptide epitopes recognized by a secondary reporter (such as leucine zipper pair sequences, binding sites for secondary antibodies, metal binding domains, epitope tags).

A CAR, a T cell expressing a CAR, an antibody, or antigen binding portion thereof, can also be conjugated with a radiolabeled amino acid. The radiolabel may be used for both diagnostic and therapeutic purposes. For instance, the radiolabel may be used to detect one or more of the antigens disclosed herein and antigen expressing cells by x-ray, emission spectra, or other diagnostic techniques. Further, the radiolabel may be used therapeutically as a toxin for treatment of tumors in a subject, for example for treatment of a neuroblastoma. Examples of labels for polypeptides include, but are not limited to, the following radioisotopes or radionucleotides: ³H, ¹⁴C, ¹⁵N, ³⁵S, ⁹⁰Y ⁹⁹Tc, ¹¹¹In, ¹²⁵I, ¹³¹I.

Means of detecting such detectable markers are well known to those of skill in the art. Thus, for example, radiolabels may be detected using photographic film or scintillation counters, fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

### Immunoresponsive Cells and Host Cells

One aspect of the present application provides an engineered immune effector cell (e.g., an immunoresponsive cell). As used herein, “an immunoresponsive cell” refers to a cell that functions in an immune response or a progenitor, or progeny thereof. In some embodiments, the immunoresponsive cell comprises the immune modulating system described herein (e.g., a cell comprising a targeting agent with specificity to a tumor associated antigen or a stress ligand; and a nucleic acid sequence encoding a polypeptide that modulates TGF-β signaling). In some embodiments, the immunoresponsive cell comprises the immune modulating system described herein (e.g., a nucleic acid sequence encoding a chimeric antigen receptor (CAR); and a nucleic acid sequence encoding a polypeptide that modulates TGF-β signaling).

In some embodiments, the immune effector cell is a T cell, an NK cell, a peripheral blood mononuclear cell (PBMC), a hematopoietic stem cell, a pluripotent stem cell, or an embryonic stem cell. In some embodiments, the immunoresponsive cell is a T cell.

For purposes herein, the T cell can be any T cell, such as a cultured T cell, e.g., a primary T cell, or a T cell from a cultured T cell line, e.g., Jurkat, SupTl, etc., or a T cell obtained from a mammal. If obtained from a mammal, the T cell can be obtained from numerous sources, including but not limited to blood, bone marrow, lymph node, the thymus, or other tissues or fluids. T cells can also be enriched for or purified. The T cell may be a human T cell. The T cell may be a T cell isolated from a human. The T cell can be any type of T cell and can be of any developmental stage, including but not limited to, CD4+/CD8+ double positive T cells, CD4+ helper T cells, e.g., Th1 and Th2 cells, CD8+ T cells (e.g., cytotoxic T cells), tumor infiltrating cells, memory T cells, memory stem cells, i.e. Tscm, naive T cells, and the like. The T cell may be a CD8+ T cell or a CD4+ T cell.

In an embodiment, the CARs as described herein can be used in suitable non-T cells. Such cells are those with an immune-effector function, such as, for example, NK cells, and T-like cells generated from pluripotent stem cells.

An embodiment further provides a host cell comprising any of the recombinant expression vectors described herein. As used herein, the term “host cell” refers to any type of cell that can contain the inventive recombinant expression vector. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5u E. coli cells, Chinese hamster ovarian cells, monkey VERO cells, COS cells, HEK293 cells, and the like. For purposes of amplifying or replicating the recombinant expression vector, the host cell may be a prokaryotic cell, e.g., a DH5a cell. For purposes of producing a recombinant CAR, the host cell may be a mammalian cell. The host cell may be a human cell. While the host cell can be of any cell type, can originate from any type of tissue, and can be of any developmental stage, the host cell may be a peripheral blood lymphocyte (PBL) or a peripheral blood mononuclear cell (PBMC). The host cell may be a T cell.

Also provided by an embodiment is a population of cells comprising at least one host cell described herein. The population of cells can be a heterogeneous population comprising the host cell comprising any of the recombinant expression vectors described, in addition to at least one other cell, e.g., a host cell (e.g., a T cell), which does not comprise any of the recombinant expression vectors, or a cell other than a T cell, e.g., a B cell, a macrophage, a neutrophil, an erythrocyte, a hepatocyte, an endothelial cell, an epithelial cell, a muscle cell, a brain cell, etc. Alternatively, the population of cells can be a substantially homogeneous population, in which the population comprises mainly host cells (e.g., consisting essentially of) comprising the recombinant expression vector. The population also can be a clonal population of cells, in which all cells of the population are clones of a single host cell comprising a recombinant expression vector, such that all cells of the population comprise the recombinant expression vector. In one embodiment of the invention, the population of cells is a clonal population comprising host cells comprising a recombinant expression vector as described herein.

CARs (including functional portions and variants thereof), nucleic acids, recombinant expression vectors, host cells (including populations thereof), and antibodies (including antigen binding portions thereof), can be isolated and/or purified. For example, a purified (or isolated) host cell preparation is one in which the host cell is more pure than cells in their natural environment within the body. Such host cells may be produced, for example, by standard purification techniques. In some embodiments, a preparation of a host cell is purified such that the host cell represents at least about 50%, for example at least about 70%, of the total cell content of the preparation. For example, the purity can be at least about 50%, can be greater than about 60%, about 70% or about 80%, or can be about 100%.

### Nucleic Acids and Expression Vectors

Further provided by an embodiment of the invention is a nucleic acid comprising a nucleotide sequence encoding any of the CARs, an antibody, or antigen binding portion thereof, described herein (including functional portions and functional variants thereof). The nucleic acids of the invention may comprise a nucleotide sequence encoding any of the leader sequences, antigen binding domains, transmembrane domains, and/or intracellular T cell signaling domains described herein.

In some embodiments, the nucleotide sequence may be codon-modified. Without being bound to a particular theory, it is believed that codon optimization of the nucleotide sequence increases the translation efficiency of the mRNA transcripts. Codon optimization of the nucleotide sequence may involve substituting a native codon for another codon that encodes the same amino acid, but can be translated by tRNA that is more readily available within a cell, thus increasing translation efficiency. Optimization of the nucleotide sequence may also reduce secondary mRNA structures that would interfere with translation, thus increasing translation efficiency.

In an embodiment of the invention, the nucleic acid may comprise a codon-modified nucleotide sequence that encodes the antigen binding domain of the inventive CAR. In another embodiment of the invention, the nucleic acid may comprise a codon-modified nucleotide sequence that encodes any of the CARs described herein (including functional portions and functional variants thereof).

Expression vectors include plasmids, retroviruses, cosmids, YACs, EBV derived episomes, and the like. A convenient vector is one that encodes a functionally complete human CH or CL immunoglobulin sequence, with appropriate restriction sites engineered so that any VH or VL sequence can be easily inserted and expressed. In such vectors, splicing usually occurs between the splice donor site in the inserted J region and the splice acceptor site preceding the human C region, and also at the splice regions that occur within the human CH exons. Suitable expression vectors can contain a number of components, for example, an origin of replication, a selectable marker gene, one or more expression control elements, such as a transcription control element (e.g., promoter, enhancer, or terminator) and/or one or more translation signals, a signal sequence or leader sequence, and the like. Polyadenylation and transcription termination occur at native chromosomal sites downstream of the coding regions. The resulting chimeric antibody may be joined to any strong promoter. Examples of suitable vectors that can be used include those that are suitable for mammalian hosts and based on viral replication systems, such as simian virus 40 (SV40), Rous sarcoma virus (RSV), adenovirus 2, bovine papilloma virus (BPV), papovavirus BK mutant (BKV), or mouse and human cytomegalovirus (CMV), and moloney murine leukemia virus (MMLV), native Ig promoters, etc. A variety of suitable vectors are known in the art, including vectors which are maintained in single copy or multiple copies, or which become integrated into the host cell chromosome, e.g., via LTRs, or via artificial chromosomes engineered with multiple integration sites (Lindenbaum et al. Nucleic Acids Res. 32:e172 (2004), Kennard et al. Biotechnol. Bioeng. Online May 20, 2009). Additional examples of suitable vectors are listed in a later section.

Thus, the invention provides one or more expression vectors comprising a nucleic acid encoding an antibody, antigen-binding fragment of an antibody (e.g., a human, humanized, chimeric antibody or antigen-binding fragment of any of the foregoing), antibody chain (e.g., heavy chain, light chain) or antigen-binding portion of an antibody chain that binds a TGFβ or TGFβR. In some embodiments, the present invention provides one or more expression vectors comprising a nucleic acid extracellular domain of TGFβR.

Expression in eukaryotic host cells is useful because such cells are more likely than prokaryotic cells to assemble and secrete a properly folded and immunologically active antibody. However, any antibody produced that is inactive due to improper folding may be renaturable according to known methods (Kim and Baldwin, “Specific Intermediates in the Folding Reactions of Small Proteins and the Mechanism of Protein Folding”, Ann. Rev. Biochem. 51, pp. 459-89 (1982)). It is possible that the host cells will produce portions of intact antibodies, such as light chain dimers or heavy chain dimers, which also are antibody homologs according to the present invention.

Also provided is a nucleic acid comprising a nucleotide sequence that is at least about 70% or more, e.g., about 80%, about 90%, about 91%, about 92%, about 93%, about 94%, about 95%, about 96%, about 97%, about 98%, or about 99% identical to any of the nucleic acids encoding a CAR construct described herein.

In an embodiment, the nucleic acids can be incorporated into a recombinant expression vector. In this regard, an embodiment provides recombinant expression vectors comprising any of the nucleic acids. For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors are not naturally-occurring as a whole.

However, parts of the vectors can be naturally-occurring. The recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring or non-naturally-occurring internucleotide linkages, or both types of linkages. Preferably, the non-naturally occurring or altered nucleotides or internucleotide linkages do not hinder the transcription or replication of the vector.

In an embodiment, the recombinant expression vector can be any suitable recombinant expression vector, and can be used to transform or transfect any suitable host cell. Suitable vectors include those designed for propagation and expansion or for expression or both, such as plasmids and viruses. The vector can be selected from the group consisting of the pUC series (Fermentas Life Sciences, Glen Burnie, Md.), the pBluescript series (Stratagene, LaJolla, Calif), the pET series (Novagen, Madison, Wis.), the pGEX series (Pharmacia Biotech, Uppsala, Sweden), and the pEX series (Clontech, Palo Alto, Calif).

Bacteriophage vectors, such as λ, λZapII (Stratagene), EMBL4, and kNMI 149, also can be used. Examples of plant expression vectors include pBIO1, pBI101.2, pBHO1.3, pBI121 and pBIN19 (Clontech). Examples of animal expression vectors include pEUK-C1, pMAM, and pMAMneo (Clontech). The recombinant expression vector may be a viral vector, e.g., a retroviral vector or a lentiviral vector. A lentiviral vector is a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include, for example, and not by way of limitation, the LENTIVECTOR® gene delivery technology from Oxford BioMedica plc, the LENTIMAX™ vector system from Lentigen and the like. Nonclinical types of lentiviral vectors are also available and would be known to one skilled in the art.

A number of transfection techniques are generally known in the art (see, e.g., Graham et al., Virology, 52: 456-467 (1973); Sambrook et al., supra; Davis et al., Basic Methods in Molecular Biology, Elsevier (1986); and Chu et al, Gene, 13: 97 (1981).

Transfection methods include calcium phosphate co-precipitation (see, e.g., Graham et al., supra), direct micro injection into cultured cells (see, e.g., Capecchi, Cell, 22: 479-488 (1980)), electroporation (see, e.g., Shigekawa et al., BioTechniques, 6: 742-751 (1988)), liposome mediated gene transfer (see, e.g., Mannino et al., BioTechniques, 6: 682-690 (1988)), lipid mediated transduction (see, e.g., Feigner et al., Proc. Natl. Acad. Sci. USA, 84: 7413-7417 (1987)), and nucleic acid delivery using high velocity microprojectiles (see, e.g., Klein et al, Nature, 327: 70-73 (1987)).

In an embodiment, the recombinant expression vectors can be prepared using standard recombinant DNA techniques described in, for example, Sambrook et al., supra, and Ausubel et al., supra. Constructs of expression vectors, which are circular or linear, can be prepared to contain a replication system functional in a prokaryotic or eukaryotic host cell. Replication systems can be derived, e.g., from ColEl, 2p plasmid, k, SV40, bovine papilloma virus, and the like.

The recombinant expression vector may comprise regulatory sequences, such as transcription and translation initiation and termination codons, which are specific to the type of host cell (e.g., bacterium, fungus, plant, or animal) into which the vector is to be introduced, as appropriate, and taking into consideration whether the vector is DNA- or RNA-based. The recombinant expression vector may comprise restriction sites to facilitate cloning.

The recombinant expression vector can include one or more marker genes, which allow for selection of transformed or transfected host cells. Marker genes include biocide resistance, e.g., resistance to antibiotics, heavy metals, etc., complementation in an auxotrophic host to provide prototrophy, and the like. Suitable marker genes for the inventive expression vectors include, for instance, neomycin/G418 resistance genes, hygromycin resistance genes, histidinol resistance genes, tetracycline resistance genes, and ampicillin resistance genes.

The recombinant expression vector can comprise a native or nonnative promoter operably linked to the nucleotide sequence encoding the CAR (including functional portions and functional variants thereof), or to the nucleotide sequence which is complementary to or which hybridizes to the nucleotide sequence encoding the CAR. The selection of promoters, e.g., strong, weak, inducible, tissue-specific and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an SV40 promoter, an RSV promoter, or a promoter found in the long-terminal repeat of the murine stem cell virus.

The recombinant expression vectors can be designed for either transient expression, for stable expression, or for both. Also, the recombinant expression vectors can be made for constitutive expression or for inducible expression.

Further, the recombinant expression vectors can be made to include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art (see, for example, Suicide Gene Therapy: Methods and Reviews, Springer, Caroline J. (Cancer Research UK Centre for Cancer Therapeutics at the Institute of Cancer Research, Sutton, Surrey, UK), Humana Press, 2004) and include, for example, the Herpes Simplex Virus (HSV) thymidine kinase (TK) gene, cytosine daminase, purine nucleoside phosphorylase, and nitroreductase.

### Methods of Treatment

The present invention relates methods of treatment comprising administering an anti-TGFβ, anti-TGFβR antigen binding molecule or an extracellular domain of a TGFβR to a subject. In some embodiments, CARs and antigen binding molecules disclosed herein can be used in methods of treating or preventing a disease in a mammal. In this regard, an embodiment provides a method of treating or preventing cancer in a mammal, comprising administering to the mammal the CARs, the nucleic acids, the recombinant expression vectors, the host cells, the population of cells, the antibodies and/or the antigen binding portions thereof, and/or the pharmaceutical compositions in an amount effective to treat or prevent cancer in the mammal.

In some embodiments, the CAR is expressed on donor cells and the anti-TGFβ, anti-TGFβR antigen binding molecule or extracellular domain of a TGFβR is secreted from these cells. In some embodiments the donor T cells for use in the T cell therapy are obtained from a patient (e.g., for an autologous T cell therapy). In other embodiments, the donor T cells for use in the T cell therapy are obtained from a subject that is not the patient (e.g., an allogeneic T cell therapy). The CAR+ T cells may be administered at a therapeutically effective amount. For example, a therapeutically effective amount of the T cells may be at least about 10⁴ cells, at least about 10⁵ cells, at least about 10⁶ cells, at least about 10⁷ cells, at least about 10⁸ cells, at least about 10′, or at least about 10¹⁰.

In some embodiments, the therapeutically effective amount of the T cells is about 10⁴ cells, about 10⁵ cells, about 10⁶ cells, about 10⁷ cells, or about 10⁸ cells. In some embodiments, the therapeutically effective amount of the CAR T cells is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 2×10⁷ cells/kg, about 3×10⁷ cells/kg, about 4×10⁷ cells/kg, about 5×10⁷ cells/kg, about 6×10⁷ cells/kg, about 7×10⁷ cells/kg, about 8×10⁷ cells/kg, or about 9×10⁷ cells/kg. In some embodiments, the therapeutically effective amount of the CAR-positive viable T cells is between about 1×10⁶ and about 2×10⁶ CAR-positive viable T cells per kg body weight up to a maximum dose of about 1×10⁸ CAR-positive viable T cells.

In some embodiments, the therapeutically effective amount of the CAR-positive viable T cells is between about 0.25×10⁶ and 2×10⁶. In some embodiments, the therapeutically effective amount of the CAR-positive viable T cells is about 0.25×10⁶, 0.3×10⁶, 0.4×10⁶, about 0.5×10⁶, about 0.6×10⁶, about 0.7×10⁶, about 0.8×10⁶, about 0.9×10⁶, about 1.0×10⁶, about 1.1×10⁶, about 1.2×10⁶, about 1.3×10⁶, about 1.4×10⁶, about 1.5×10⁶, about 1.6×10⁶, about 1.7×10⁶, about 1.8×10⁶, about 1.9×10⁶, or about 2.0×10⁶ CAR-positive viable T cells.

In some embodiments, the therapeutically effective amount of the CAR-positive viable T cells is between about 0.4×10⁸ and about 2×10⁸ CAR-positive viable T cells. In some embodiments, the therapeutically effective amount of the CAR-positive viable T cells is about 0.4×10⁸, about 0.5×10⁸, about 0.6×10⁸, about 0.7×10⁸, about 0.8×10⁸, about 0.9×10⁸, about 1.0×10⁸, about 1.1×10⁸, about 1.2×10⁸, about 1.3×10⁸, about 1.4×10⁸, about 1.5×10⁸, about 1.6×10⁸, about 1.7×10⁸, about 1.8×10⁸, about 1.9×10⁸, or about 2.0×10⁸ CAR-positive viable T cells.

An embodiment further comprises lymphodepleting the mammal prior to administering the CARs disclosed herein. Examples of lymphodepletion include, but may not be limited to, nonmyeloablative lymphodepleting chemotherapy, myeloablative lymphodepleting chemotherapy, total body irradiation, etc.

For purposes of the methods, wherein host cells or populations of cells are administered, the cells can be cells that are allogeneic or autologous to the mammal. In some embodiments, the cells are autologous to the mammal. In some embodiments, the cells are allogenic to the mammal. As used herein, allogeneic means any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically. As used herein, “autologous” means any material derived from the same individual to whom it is later to be re-introduced into the individual.

The mammal referred to herein can be any mammal. As used herein, the term “mammal” refers to any mammal, including, but not limited to, mammals of the order Rodentia, such as mice and hamsters, and mammals of the order Logomorpha, such as rabbits. The mammals may be from the order Camivora, including Felines (cats) and Canines (dogs). The mammals may be from the order Artiodactyla, including Bovines (cows) and Swines (pigs) or of the order Perssodactyla, including Equines (horses). The mammals may be of the order Primates, Ceboids, or Simoids (monkeys) or of the order Anthropoids (humans and apes). In some embodiments, the mammal is a human.

With respect to the methods, the cancer can be any cancer, including any of acute lymphocytic cancer, acute myeloid leukemia, alveolar rhabdomyosarcoma, bladder cancer (e.g., bladder carcinoma), bone cancer, brain cancer (e.g., meduloblastoma), breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, fibrosarcoma, gastrointestinal carcinoid tumor, head and neck cancer (e.g., head and neck squamous cell carcinoma), Hodgkin lymphoma, hypopharynx cancer, kidney cancer, larynx cancer, leukemia, liquid tumors, liver cancer, lung cancer (e.g., non-small cell lung carcinoma and lung adenocarcinoma), lymphoma, mesothelioma, mastocytoma, melanoma, multiple myeloma, nasopharynx cancer, non-Hodgkin lymphoma, B-chronic lymphocytic leukemia, hairy cell leukemia, acute lymphocytic leukemia (ALL), and Burkitt's lymphoma, ovarian cancer, pancreatic cancer, peritoneum, omentum, and mesentery cancer, pharynx cancer, prostate cancer, rectal cancer, renal cancer, skin cancer, small intestine cancer, soft tissue cancer, solid tumors, synovial sarcoma, gastric cancer, testicular cancer, thyroid cancer, and ureter cancer.

In certain embodiments, the cancer is a gastrointestinal cancer. In some embodiments, the cancer is gastric cancer. In some embodiments, the cancer is colorectal cancer. In some embodiments, the cancer is colon cancer. In some embodiments, the cancer has abnormal expression of TGFβ or abnormal TGFβ signaling.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the methods can provide any amount or any level of treatment or prevention of cancer in a mammal.

Furthermore, the treatment or prevention provided by the method can include treatment or prevention of one or more conditions or symptoms of the disease, e.g., cancer, being treated or prevented. Also, for purposes herein, “prevention” can encompass delaying the onset of the disease, or a symptom or condition thereof.

Methods of testing a CAR for the ability to recognize target cells and for antigen specificity are known in the art. For instance, Clay et al., J. Immunol, 163: 507-513 (1999), teaches methods of measuring the release of cytokines (e.g., interferon-γ, granulocyte/monocyte colony stimulating factor (GM-CSF), tumor necrosis factor a (TNF-α) or interleukin 2 (IL-2)). In addition, CAR function can be evaluated by measurement of cellular cytotoxicity, as described in Zhao et al, J. Immunol, 174: 4415-4423 (2005).

Another embodiment provides for the use of the CARs, nucleic acids, recombinant expression vectors, host cells, populations of cells, antibodies, or antigen binding portions thereof, and/or pharmaceutical compositions of the invention, for the treatment or prevention of a proliferative disorder, e.g., cancer, in a mammal. The cancer may be any of the cancers described herein.

Any method of administration can be used for the disclosed therapeutic agents, including local and systemic administration. For example topical, oral, intravascular such as intravenous, intramuscular, intraperitoneal, intranasal, intradermal, intrathecal and subcutaneous administration can be used. The particular mode of administration and the dosage regimen will be selected by the attending clinician, taking into account the particulars of the case (for example the subject, the disease, the disease state involved, and whether the treatment is prophylactic). In cases in which more than one agent or composition is being administered, one or more routes of administration may be used; for example, a chemotherapeutic agent may be administered orally and an antibody or antigen binding fragment or conjugate or composition may be administered intravenously. Methods of administration include injection for which the CAR, CAR T Cell, conjugates, antibodies, antigen binding fragments, or compositions are provided in a nontoxic pharmaceutically acceptable carrier such as water, saline, Ringer's solution, dextrose solution, 5% human serum albumin, fixed oils, ethyl oleate, or liposomes. In some embodiments, local administration of the disclosed compounds can be used, for instance by applying the antibody or antigen binding fragment to a region of tissue from which a tumor has been removed, or a region suspected of being prone to tumor development. In some embodiments, sustained intra-tumoral (or near-tumoral) release of the pharmaceutical preparation that includes a therapeutically effective amount of the antibody or antigen binding fragment may be beneficial. In other examples, the conjugate is applied as an eye drop topically to the cornea, or intravitreally into the eye.

The disclosed therapeutic agents can be formulated in unit dosage form suitable for individual administration of precise dosages. In addition, the disclosed therapeutic agents may be administered in a single dose or in a multiple dose schedule. A multiple dose schedule is one in which a primary course of treatment may be with more than one separate dose, for instance 1-10 doses, followed by other doses given at subsequent time intervals as needed to maintain or reinforce the action of the compositions. Treatment can involve daily or multi-daily doses of compound(s) over a period of a few days to months, or even years. Thus, the dosage regime will also, at least in part, be determined based on the particular needs of the subject to be treated and will be dependent upon the judgment of the administering practitioner.

Typical dosages of the antibodies or conjugates can range from about 0.01 to about 30 mg/kg, such as from about 0.1 to about 10 mg/kg.

In particular examples, the subject is administered a therapeutic composition that includes one or more of the conjugates, antibodies, compositions, CARs, CAR T cells or additional agents, on a multiple daily dosing schedule, such as at least two consecutive days, 10 consecutive days, and so forth, for example for a period of weeks, months, or years. In one example, the subject is administered the conjugates, antibodies, compositions or additional agents for a period of at least 30 days, such as at least 2 months, at least 4 months, at least 6 months, at least 12 months, at least 24 months, or at least 36 months.

In some embodiments, the disclosed methods include providing surgery, radiation therapy, and/or chemotherapeutics to the subject in combination with a disclosed antibody, antigen binding fragment, conjugate, CAR or T cell expressing a CAR (for example, sequentially, substantially simultaneously, or simultaneously). Methods and therapeutic dosages of such agents and treatments are known to those skilled in the art, and can be determined by a skilled clinician. Preparation and dosing schedules for the additional agent may be used according to manufacturer's instructions or as determined empirically by the skilled practitioner. Preparation and dosing schedules for such chemotherapy are also described in Chemotherapy Service, (1992) Ed., M. C. Perry, Williams & Wilkins, Baltimore, Md.

In some embodiments, the combination therapy can include administration of a therapeutically effective amount of an additional cancer inhibitor to a subject. Non-limiting examples of additional therapeutic agents that can be used with the combination therapy include microtubule binding agents, DNA intercalators or cross-linkers, DNA synthesis inhibitors, DNA and RNA transcription inhibitors, antibodies, enzymes, enzyme inhibitors, gene regulators, and angiogenesis inhibitors. These agents (which are administered at a therapeutically effective amount) and treatments can be used alone or in combination. For example, any suitable anti-cancer or anti-angiogenic agent can be administered in combination with the CARs, CAR-T cells, antibodies, antigen binding fragment, or conjugates disclosed herein. Methods and therapeutic dosages of such agents are known to those skilled in the art, and can be determined by a skilled clinician.

Additional chemotherapeutic agents include, but are not limited to alkylating agents, such as nitrogen mustards (for example, chlorambucil, chlormethine, cyclophosphamide, ifosfamide, and melphalan), nitrosoureas (for example, carmustine, fotemustine, lomustine, and streptozocin), platinum compounds (for example, carboplatin, cisplatin, oxaliplatin, and BBR3464), busulfan, dacarbazine, mechlorethamine, procarbazine, temozolomide, thiotepa, and uramustine; antimetabolites, such as folic acid (for example, methotrexate, pemetrexed, and raltitrexed), purine (for example, cladribine, clofarabine, fludarabine, mercaptopurine, and tioguanine), pyrimidine (for example, capecitabine), cytarabine, fluorouracil, and gemcitabine; plant alkaloids, such as podophyllum (for example, etoposide, and teniposide), taxane (for example, docetaxel and paclitaxel), vinca (for example, vinblastine, vincristine, vindesine, and vinorelbine); cytotoxic/antitumor antibiotics, such as anthracycline family members (for example, daunorubicin, doxorubicin, epirubicin, idarubicin, mitoxantrone, and valrubicin), bleomycin, rifampicin, hydroxyurea, and mitomycin; topoisomerase inhibitors, such as topotecan and irinotecan; monoclonal antibodies, such as alemtuzumab, bevacizumab, cetuximab, gemtuzumab, rituximab, panitumumab, pertuzumab, and trastuzumab; photosensitizers, such as aminolevulinic acid, methyl aminolevulinate, porfimer sodium, and verteporfin; and other agents, such as alitretinoin, altretamine, amsacrine, anagrelide, arsenic trioxide, asparaginase, axitinib, bexarotene, bevacizumab, bortezomib, celecoxib, denileukin diftitox, erlotinib, estramustine, gefitinib, hydroxycarbamide, imatinib, lapatinib, pazopanib, pentostatin, masoprocol, mitotane, pegaspargase, tamoxifen, sorafenib, sunitinib, vemurafinib, vandetanib, and tretinoin. Selection and therapeutic dosages of such agents are known to those skilled in the art, and can be determined by a skilled clinician.

The combination therapy may provide synergy and prove synergistic, that is, the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation, a synergistic effect may be attained when the compounds are administered or delivered sequentially, for example by different injections in separate syringes. In general, during alternation, an effective dosage of each active ingredient is administered sequentially, i.e. serially, whereas in combination therapy, effective dosages of two or more active ingredients are administered together.

In various embodiments, the immune modulating system comprising a TGFβ signaling modulator described herein may be included in a course of treatment that further includes administration of at least one additional agent to a subject. In various embodiments, an additional agent administered in combination with the immune modulating system comprising the TGFβ signaling modulator as described herein may be chemotherapy agent. In various embodiments, an additional agent administered in combination with an antigen binding agent as described herein may be an agent that inhibits inflammation.

In some embodiments, the TGFβ signaling modulator is a single domain antibody or a secretable scFv with specificity for human TGFβ. In some embodiments, the TGFβ signaling modulator is a single domain antibody or a secretable scFv with specificity for a human TGFβR. In some embodiments, the TGFβ signaling modulator can be conjugated (e.g., linked to) to a therapeutic agent (e.g., a chemotherapeutic agent and a radioactive atom) for binding to a cancer cell, delivering therapeutic agent to the cancer cell, and killing the cancer cell which expresses human TGFβ. In some embodiments, TGFβ signaling modulator is linked to a therapeutic agent. In some embodiments, therapeutic agent is a chemotherapeutic agent, a cytokine, a radioactive atom, an siRNA, or a toxin. In some embodiments, therapeutic agent is a chemotherapeutic agent. In some embodiments, the agent is a radioactive atom.

In some embodiments, the methods can be performed in conjunction with other therapies for disorders with abnormal TGFβ signaling. For example, the composition can be administered to a subject at the same time, prior to, or after, chemotherapy. In some embodiments, the composition can be administered to a subject at the same time, prior to, or after, an adoptive therapy method.

In various embodiments, an additional agent administered in combination with the immune modulating system comprising a TGFβ signaling modulator as described herein, may be administered at the same time as the TGFβ signaling modulator, on the same day, or in the same week. In various embodiments, an additional agent administered in combination with the TGFβ signaling modulator as described herein may be administered in a single formulation with the immune modulating system. In certain embodiments, an additional agent administered in a manner temporally separated from administration TGFβ signaling modulator as described herein, e.g., one or more hours before or after, one or more days before or after, one or more weeks before or after, or one or more months before or after administration of the TGFβ signaling modulator. In various embodiments, the administration frequency of one or more additional agents may be the same as, similar to, or different from the administration frequency of the TGFβ signaling modulator as described herein.

In some embodiments, compositions can be formulated with one or more additional therapeutic agents, e.g., additional therapies for treating or preventing a TGFβ-associated disorder (e.g., a cancer or autoimmune disorder) in a subject. Additional agents for treating a TGFβ-associated disorder in a subject will vary depending on the particular disorder being treated, but can include, without limitation, rituximab, cyclophosphamide, doxorubicin, vincristine, prednisone, osfamide, carboplatin, etoposide, dexamethasone, cytarabine, cisplatin, cyclophosphamide, or fludarabine.

### Compositions

Compositions are provided herein for use in gene therapy, immunotherapy and/or cell therapy that include one or more of the disclosed CARs, or T cells expressing a CAR, antibodies, antigen binding fragments, conjugates, CARs, or T cells expressing a CAR that specifically bind to one or more antigens disclosed herein, in a carrier (such as a pharmaceutically acceptable carrier). The compositions can be prepared in unit dosage forms for administration to a subject. The amount and timing of administration are at the discretion of the treating clinician to achieve the desired outcome. The compositions can be formulated for systemic (such as intravenous) or local (such as intra-tumor) administration. In one example, a disclosed CARs, or T cells expressing a CAR, antibody, antigen binding fragment, conjugate, is formulated for parenteral administration, such as intravenous administration. Compositions including a CAR, or T cell expressing a CAR, a conjugate, antibody or antigen binding fragment as disclosed herein are of use, for example, for the treatment and detection of a tumor, for example, and not by way of limitation, a neuroblastoma. In some examples, the compositions are useful for the treatment or detection of a carcinoma. The compositions including a CAR, or T cell expressing a CAR, a conjugate, antibody or antigen binding fragment as disclosed herein are also of use, for example, for the detection of pathological angiogenesis.

The compositions for administration can include a solution of the CAR, or T cell expressing a CAR, conjugate, antibody or antigen binding fragment dissolved in a pharmaceutically acceptable carrier, such as an aqueous carrier. A variety of aqueous carriers can be used, for example, buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents, adjuvant agents, and the like, for example, sodium acetate, sodium chloride, potassium chloride, calcium chloride, sodium lactate and the like. The concentration of a CAR, or T cell expressing a CAR, antibody or antigen binding fragment or conjugate in these formulations can vary widely, and will be selected primarily based on fluid volumes, viscosities, body weight and the like in accordance with the particular mode of administration selected and the subject's needs. Actual methods of preparing such dosage forms for use in gene therapy, immunotherapy and/or cell therapy are known, or will be apparent, to those skilled in the art.

A typical composition for intravenous administration includes about 0.01 to about 30 mg/kg of antibody or antigen binding fragment or conjugate per subject per day (or the corresponding dose of a CAR, or T cell expressing a CAR, conjugate including the antibody or antigen binding fragment). Actual methods for preparing administrable compositions will be known or apparent to those skilled in the art and are described in more detail in such publications as Remington's Pharmaceutical Science, 19th ed., Mack Publishing Company, Easton, Pa. (1995).

Controlled release parenteral formulations can be made as implants, oily injections, or as particulate systems. For a broad overview of protein delivery systems see, Banga, A. J., Therapeutic Peptides and Proteins: Formulation, Processing, and Delivery Systems, Technomic Publishing Company, Inc., Lancaster, Pa., (1995). Particulate systems include microspheres, microparticles, microcapsules, nanocapsules, nanospheres, and nanoparticles. Microcapsules contain the therapeutic protein, such as a cytotoxin or a drug, as a central core. In microspheres, the therapeutic is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly. See, for example, Kreuter, J., Colloidal Drug Delivery Systems, J. Kreuter, ed., Marcel Dekker, Inc., New York, N.Y., pp. 219-342 (1994); and Tice & Tabibi, Treatise on Controlled Drug Delivery, A. Kydonieus, ed., Marcel Dekker, Inc. New York, N.Y., pp. 315-339, (1992).

Polymers can be used for ion-controlled release of the CARs, or T cells expressing a CAR, antibody or antigen binding fragment or conjugate compositions disclosed herein. Various degradable and nondegradable polymeric matrices for use in controlled drug delivery are known in the art (Langer, Accounts Chem. Res. 26:537-542, 1993). For example, the block copolymer, polaxamer 407, exists as a viscous yet mobile liquid at low temperatures but forms a semisolid gel at body temperature. It has been shown to be an effective vehicle for formulation and sustained delivery of recombinant interleukin-2 and urease (Johnston et al., Pharm. Res. 9:425-434, 1992; and Pec et al., J. Parent. Sci. Tech. 44(2):58-65, 1990). Alternatively, hydroxyapatite has been used as a microcarrier for controlled release of proteins (Ijntema et al., Int. J. Pharm. 112:215-224, 1994). In yet another aspect, liposomes are used for controlled release as well as drug targeting of the lipid-capsulated drug (Betageri et al., Liposome Drug Delivery Systems, Technomic Publishing Co., Inc., Lancaster, Pa. (1993)). Numerous additional systems for controlled delivery of therapeutic proteins are known (see U.S. Pat. Nos. 5,055,303; 5,188,837; 4,235,871; 4,501,728; 4,837,028; 4,957,735; 5,019,369; 5,055,303; 5,514,670; 5,413,797; 5,268,164; 5,004,697; 4,902,505; 5,506,206; 5,271,961; 5,254,342 and 5,534,496).

### Kits

In one aspect, kits employing the CARs disclosed herein are also provided. For example, kits for treating a tumor in a subject, or making a CAR T cell that expresses one or more of the CARs disclosed herein. The kits will typically include a disclosed antibody, antigen binding fragment, conjugate, nucleic acid molecule, CAR or T cell expressing a CAR as disclosed herein. More than one of the disclosed antibodies, antigen binding fragments, conjugates, nucleic acid molecules, CARs or T cells expressing a CAR can be included in the kit.

The kit can include a container and a label or package insert on or associated with the container. Suitable containers include, for example, bottles, vials, syringes, etc. The containers may be formed from a variety of materials such as glass or plastic. The container typically holds a composition including one or more of the disclosed antibodies, antigen binding fragments, conjugates, nucleic acid molecules, CARs or T cells expressing a CAR. In several embodiments the container may have a sterile access port (for example the container may be an intravenous solution bag or a vial having a stopper pierceable by a hypodermic injection needle). A label or package insert indicates that the composition is used for treating the particular condition.

The label or package insert typically will further include instructions for use of a disclosed antibodies, antigen binding fragments, conjugates, nucleic acid molecules, CARs or T cells expressing a CAR, for example, in a method of treating or preventing a tumor or of making a CAR T cell. The package insert typically includes instructions customarily included in commercial packages of therapeutic products that contain information about the indications, usage, dosage, administration, contraindications and/or warnings concerning the use of such therapeutic products. The instructional materials may be written, in an electronic form (such as a computer diskette or compact disk) or may be visual (such as video files). The kits may also include additional components to facilitate the particular application for which the kit is designed. Thus, for example, the kit may additionally contain means of detecting a label (such as enzyme substrates for enzymatic labels, filter sets to detect fluorescent labels, appropriate secondary labels such as a secondary antibody, or the like). The kits may additionally include buffers and other reagents routinely used for the practice of a particular method. Such kits and appropriate contents are well known to those of skill in the art.

Unless stated otherwise, all technical and scientific terms and phrases used herein have the same meaning as commonly understood by one of ordinary skill in the art. Although any methods and materials similar or equivalent to those described herein can be used in the practice or testing of the present invention, the preferred methods and materials are now described. All publications mentioned herein are incorporated herein by reference.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)), which is incorporated herein by reference for any purpose.

All publications, patent applications, patents and other references mentioned herein are incorporated by reference in their entirety. The present invention will be more fully understood by reference to the following Examples.

### EXAMPLES

These Examples are set forth to aid in the understanding of the invention but are not intended to, and should not be construed to limit its scope in any way. The Examples do not include detailed descriptions of conventional methods that would be well known to those of ordinary skill in the art (molecular cloning techniques, etc.).

### Example 1. Immunoresponsive Cells Co-Expressing a Chimeric Antigen Receptor (CAR) and a TGF-B Signaling Modulator

This example illustrates co-expression of a TGF-β signaling modulator and a CAR using an immune modulating system in human T cells. Immune modulating constructs encoding a TGF-B signaling modulator (e.g., anti-TGFβ and anti-TGFβR2) and an anti-human CD19 CAR (SJ25C1 extracellular antigen binding domain) were packaged for retroviral delivery. Phoenix A Retroviral Packaging Cell Line (ATCC) was grown to 50-70% confluency in DMEM 20% FBS and Pen/Strep. DNA complexes were prepared using the respective plasmids encoding the TGF-B signaling modulator and the CAR construct, helper plasmids gag-pol and pVSVG and the transduction reagent Fugene HD (Promega) according to manufacturers protocol. 20-48 hrs after transfection, virus supernatant was harvested, aliquoted and frozen for further use.

Human PBMCs were isolated from Leukopaks using a density gradient and frozen until further use. Human T cells were isolated by magnetic selection (T cell isolation kit; Stemcell) from previously frozen PBMCs. Purified human T cells were cultured for 2 days in complete Optimizer medium (Optimizer basal medium (ThermoFisher #A10221-01)+26 ml OptiMizer supplement (ThermoFisher #A10484-02)+20 ml ICSR (CTS Immune Cell SR), ThermoFisher #A25961-01)+10 ml of 200 mM L-glutamine, (Gibco 25030-081)+PenStrep, (Gibco 15140-122)) containing 2 ng/ml human IL-2 (Miltenyi) and T cell Transact beads (Miltenyi)).

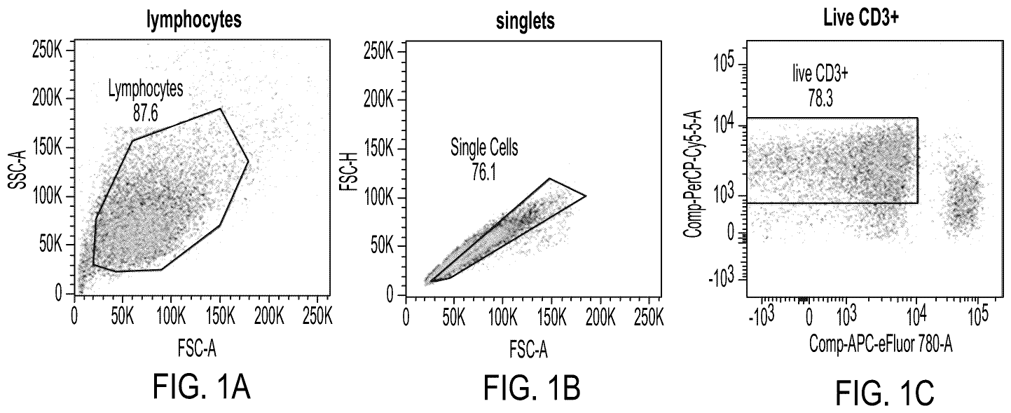

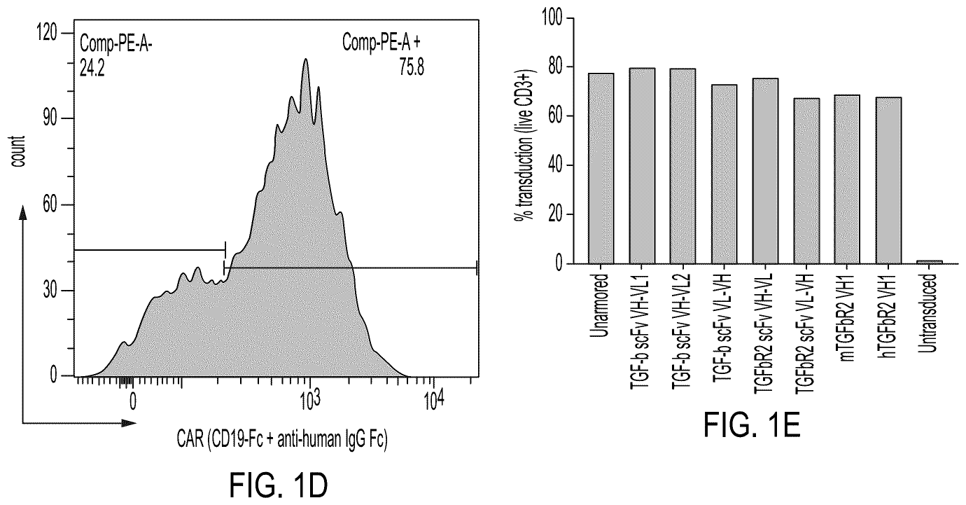

T cells were transferred to retronectin-coated plates (Takara; 40 ug/ml retronectin) and spin-transduced with the appropriate volume of virus. Transduction was confirmed and quantified by flow cytometry at different time points. In brief, cells were incubated with 250 ng hCD19-hFc protein (RnD Systems) or in house produced hGCC-Fc protein in FACS buffer for 1 hour at 4′C. After a wash with FACS buffer the cells were resuspended with a secondary antibody against human FC (Biolegend) for 20 minutes at room temperature. In some experiments antibodies against CD4, CD8 or other surface markers were added. Dead cells were excluded from the analysis using fixable viability dye (Thermofisher). Cells were fixed in PBS 2% FCS 4% Formaldehyde before analysis by flow cytometry (FACS Fortessa, BD Biosciences). Transduction efficiencies are displayed as % live cells positive for CAR staining. Flow cytometry results showed populations of 87.6% lymphocytes, 76.1% singlets, 78.3% live CD3+ cells and 75.8% cells showed CAR expression (FIG. 1A-1D). Transduction efficiencies are shown as % live cells positive for CAR staining (FIG. 1E).

### Example 2. In Vitro Killing Using TGF-β Modulating Human CAR-T Cells

This example illustrates in vitro killing by human CAR-T cells co-expressing a TGF-β signaling modulator. In vitro killing by armored human CAR-T cells is comparable to unarmored CAR-T cells

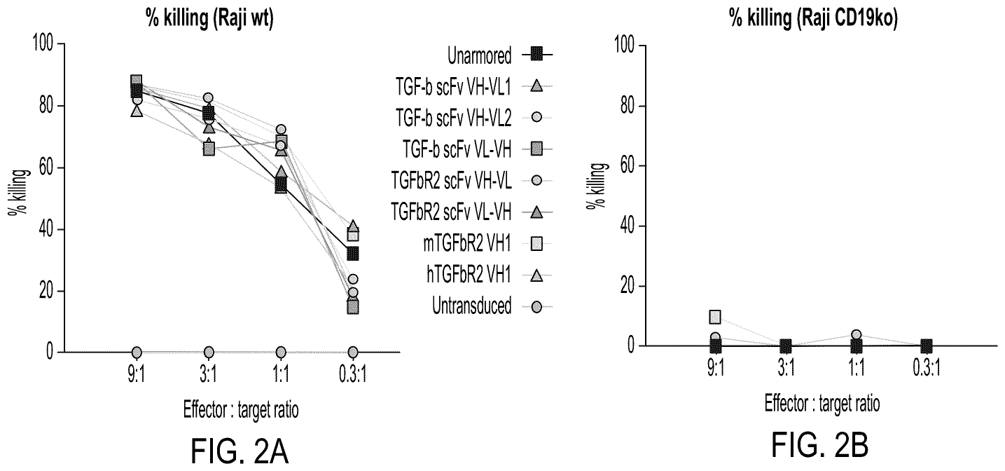

Raji (ATCC CD19 positive) or Raji CD19ko (negative for human CD19) cells were stained with proliferation dye efluor 450 (Thermofisher) according to manufacturer's protocol and plated into 96 well plates at least 2 hours before the addition of TGF-β modulating CAR-T cells described in Example 1. CAR-T cells were added at effector:target ratios 0:1, 0.3:1, 1:1, 3:1, 9:1 and T cells alone were incubated overnight at 37° C. The following day FACS staining was performed using fluorochrome conjugated antibodies against human CD107a (LAMP-1) (Biolegend), TCR α/β (Biolegend), and human CD4 antibody (Biolegend). Cells were incubated with antibodies for 30 minutes at 4° C., washed with PBS and stained with fixable viability dye (Thermofisher) according to manufacturer's protocol. Ccells were washed with 1× Annexin V Binding Buffer (Biolegend) and stained with Annexin V FITC. Cells were fixed in cytofix (BD Biosciences) before acquisition on a FACS Fortessa (BD Biosciences). TGF-β modulating CD19 CAR-T cells, demonstrated target specific in vitro killing against CD19 positive Raji cells (FIG. 2A) but not against CD19 negative control cells (Raji CD19ko) (FIG. 2B).

### Example 3. Secretion of TGF-β Modulators from Immunoresponsive Cells

This example demonstrates that TGF-β modulating CAR-T cells secrete co-expressed TGF-β modulators (e.g., anti-TGF-β that binds TGF-β and anti-TGFβR2 that binds TGFβR2).

Supernatant from TGF-β modulating CAR-T cells were assayed by ELISA to detect anti-TGF-β and anti-TGFβR2 antibodies. Maxisorp 96 well plates were coated with recombinant human TGF-β (4; RnD System μg/ml) or hTGFβR2-Fc (0.1 mg/ml; RnD System) in 100 μl coating buffer overnight at 4° C. Plates were washed with 1× wash buffer and blocked for 1 hour with reagent diluent at room temperature. CAR-T supernatant, recombinant TGFβR2-flag, or recombinant TGF-3-flag antibody was added and incubated for 2 hours at room temperature.

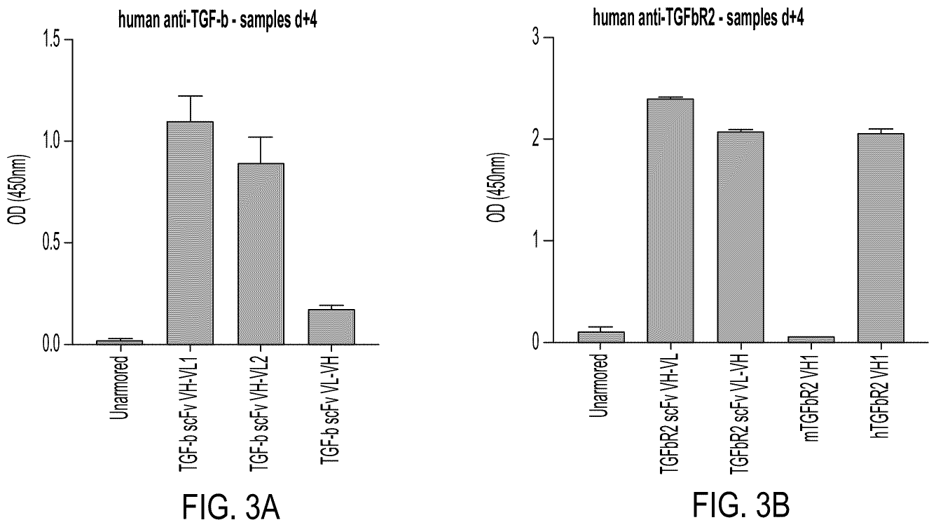

After another washing step, HRP-conjugated flag-tag antibody was added and incubated for 30 minutes at room temperature. Plates were washed and TMB substrate was added for 10-20 minutes. The reaction was stopped using Stop reagent and plates were read at 450 nm using a Pherastar Plate reader. ELISA using coated TGF-b detected high levels of TGF-b scFv VH-VL1 and TGF-b scFv VH-VL2 as compared to TGF-b scFv VL-VH using anti-flag tag HRP antibodies for binder detection (FIG. 3A). ELISA using coated TGFbR2-Fc detected high levels of TGFbR2 scFv VH-VL, TGFbR2 scFv VL-VH and hTGFbR2 VH1 from human CAR-T but was unable to detect mTGFbR2 VH1 (FIG. 3B). TGF-β binders and TGFβR2 binders were secreted by TGF-β modulating CAR-T cells and bind to their cognate antigen.

### Example 4. Human CAR-T Cells Secrete Neutralizing Antibodies Against TGF-β/TGFβR2

This example illustrates the presence of neutralizing antibodies against TGF-β/TGFβR2 in TGF-β modulating CAR-T supernatants.

Functional assessment of TGF-β blocking binders in the supernatant of CAR-T cells was performed using SBE-Luc reporter cells (HEK293 cells expressing firefly luciferase under control of the Smad Binding Element (SBE) (BPS Biosciences)), designed for monitoring the activity of the TGF-β/SMAD signaling pathway. TGF-β proteins bind to their cognate receptors on the cell surface, initiating a signaling cascade that leads to phosphorylation and activation of SMAD2 and SMAD3, which then form a complex with SMAD4. The SMAD complex translocates to the nucleus and binds to the SMAD binding element (SBE), leading to transcription and expression of TGF-β/SMAD responsive genes. The presence of blocking binders was detected by their ability to inhibit TGF-β induced luciferase expression in SBE-Luc reporter cells. An exemplary assay to evaluate potency to inhibit TGF-β-induced reporter activity was performed as follows.

SBE-Luc cells were seeded into Poly-D Lysine coated 96-well plates at a concentration of 1×10⁵ cells/well in 100 μl of fresh media (X-VIVO15 containing 1× Penn/Strep) and incubated for 4 hours at 37° C. and 5% CO₂. Supernatant from CAR-T cells or dilutions thereof was mixed with an equivalent volume of TGF-β (4 ng/ml in X-VIVO15) and incubated at room temperature for 15 minutes to allow the TGF-β to complex with TGF-b contained in the CAR-T supernatant. 100 μl of the mix was added to the SBE-Luc reporter cells in duplicates and incubated overnight at 37° C. and 5% CO₂. The final concentration of TGF-β was 1 ng/ml. Each experiment included a titration curve of escalating dilutions of TGF-β antibody (1D11 (BioXcell) or TGF-β binders or TGFβR2 binders) in the presence of 1 ng/ml TGF-β.

The following day 100 μl culture supernatant was removed and 100 μl Luciferin-D containing detection reagent (ONE-Step™ Luciferase Assay System) was added. Cells were resuspended and transferred to a white detection plate and luminescence was measured using Pherastar plate reader. Luciferase activity was recorded as CPM. Data was analyzed using MS Excel or Graphpad prism. Nonlinear regression fit was performed using Sigmoidal dose-response (variable slope) of Graphpad prism. IC50 values were calculated.

Inhibitory activity (%) was calculated using the following equation:

Inhibition (%)=(1−CPM of sample/CPM max of TGF-β (1 ng/ml) treated sample)×100

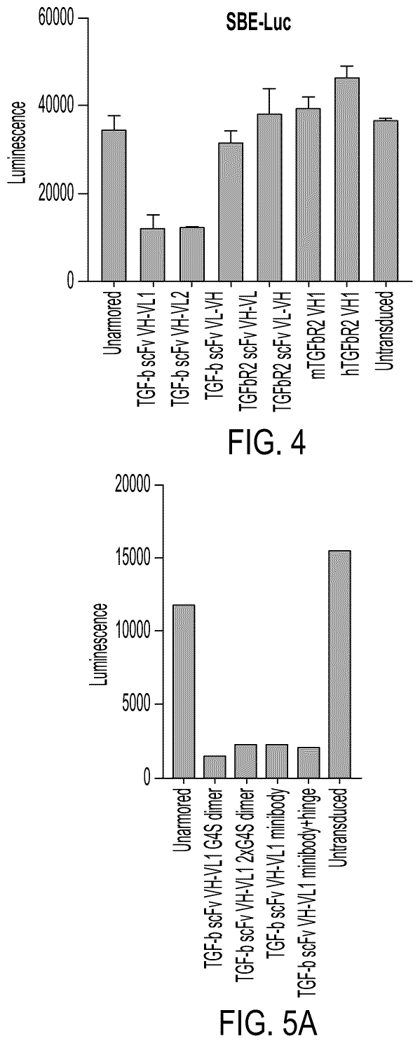

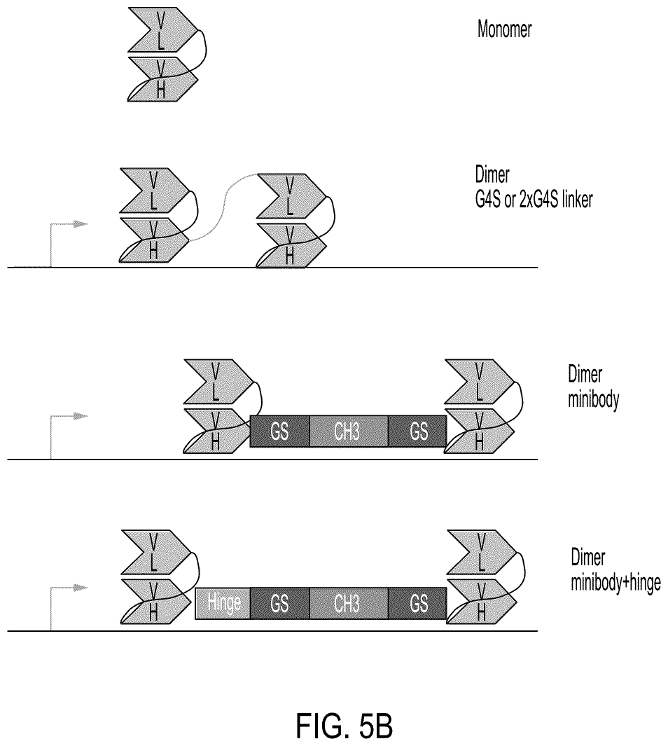

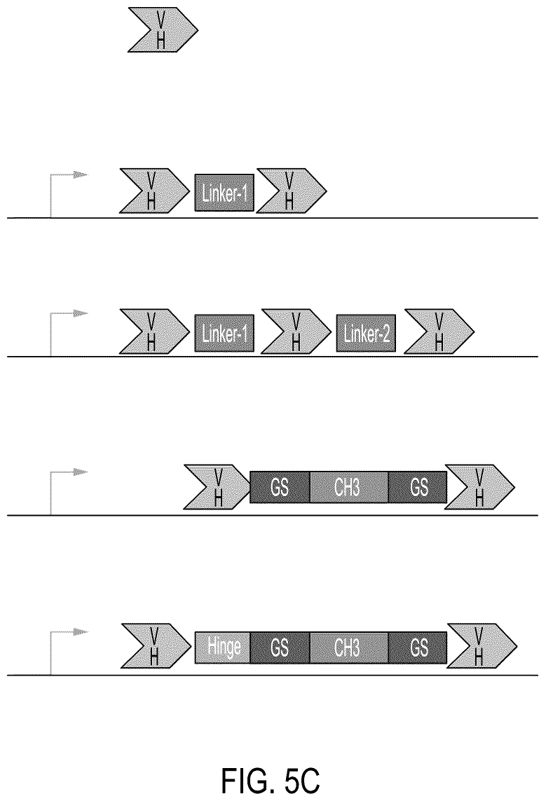

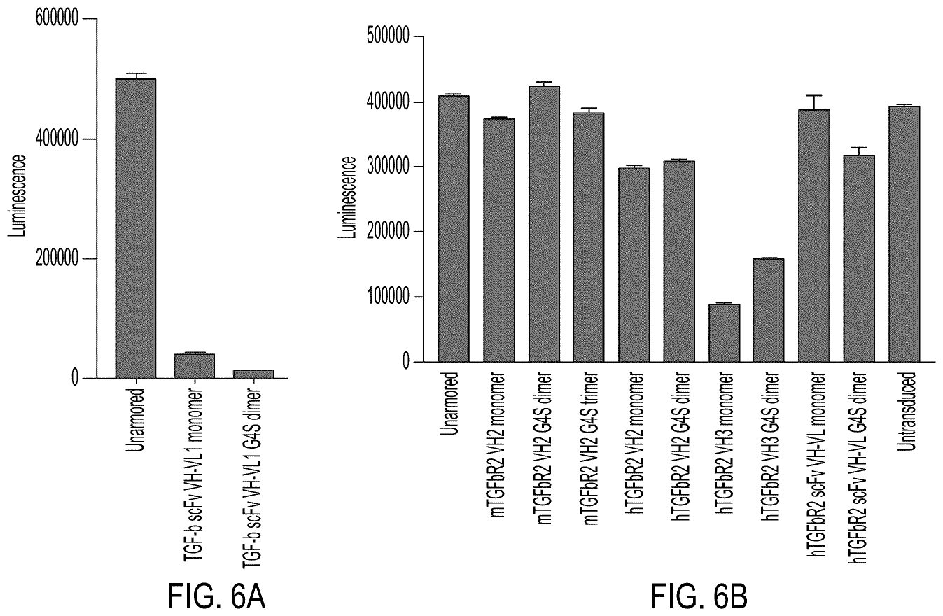

The results showed that supernatant from CAR-T cells secreting constructs TGF-β scFv VH-VL1 (SEQ ID NO: 1) and TGF-β scFv VH-VL2 (SEQ ID NO: 2) inhibits TGF-β signaling (FIG. 4). Additional constructs were designed and screened using a luciferase reporter assay for the secretion of multimeric binders against TGF-β or TGFβR2 (FIG. 5 and FIG. 6). TGF-β modulating CAR-T cells that secreted multimeric antibodies to TGF-β and TGFβR2 were identified. Multimeric TGF-b binders can be secreted by human CAR-T cells and inhibit TGF-b signaling regardless of the linker. Four different linkers were analyzed as shown in the figure below. Similar results were observed using human anti-GCC CAR-T cells (data not shown).

### Example 5. TGF-B Modulating CAR-T Cells Secrete Multimeric Binders Against TGF-β or TGFβR2

This example illustrates screening and identification of mouse CAR-T cells that secrete multimeric binders against TGF-β or TGFβR2. To generate mouse CAR-T cells, Platinum-E Retroviral Packaging Cell Line was grown to 50-70% confluency in DMEM 20% FBS and Pen/Strep. DNA complexes were prepared using the immune modulating system plasmids encoding the CAR construct and TGFB modulator (e.g., anti-TGF-b scFv monomer, anti-TGF-b scFv dimer), a packaging construct and the transduction reagent Fugene HD according to manufacturer's protocol. The solution was mixed and incubated at room temperature for 10 minutes and 850 μl of complex was added per 10 cm² dish of cells. Mouse T cells were isolated by magnetic selection using a T cell isolation kit from the spleens of Balb/c or C57BL/6 mice, respectively. Purified mouse T cells were cultured for 2 days with mouse T cell activator beads (1:1 ratio) in RPMI 10% heat inactivated FCS, Pen/Strep and mouse IL-2 (30 U/ml). Virus was harvested about 48 hours after transfection and filtered through a 0.4 μm syringe filter. T cells were transferred to retronectin (coated with 40 μg/ml retronectin according to manufacturer's protocol) coated plates and spin-transduced with the appropriate volume of virus. Transduction was confirmed and quantified by flow cytometry at different time points.

Cells were incubated with 250 ng hCD19-hFc protein in FACS buffer for 1 hour at 4° C. After a wash with FACS buffer, the cells were resuspended with a secondary antibody against human FC for 20 minutes at room temperature. In some experiments, antibodies against CD4, CD8 or other surface markers were added. Dead cells were excluded from the analysis using fixable viability dye. Cells were fixed in PBS 2% FCS, 4% formaldehyde before analysis by flow cytometry (FACS Fortessa). Transduction efficiencies are displayed as percent (%) live cells positive for CAR staining.

Flow cytometry results showed the relative proportion of unarmored T cells, TGF-β monomer, TGF-β dimer and untransduced cells. Supernatant from mouse CAR-T cells harvested d+2 after transduction was probed for inhibition of TGF-b signaling in the SBE-Luc TGF-b reporter assay (method as described in Example 4). Supernatant from mouse CAR-T secreting TGF-b scFv monomer and dimer inhibited TGF-b signaling based on the luciferase reporter activity. Mouse CAR-T cells that secreted multimeric antibodies to TGF-β and TGFβR2 were identified.

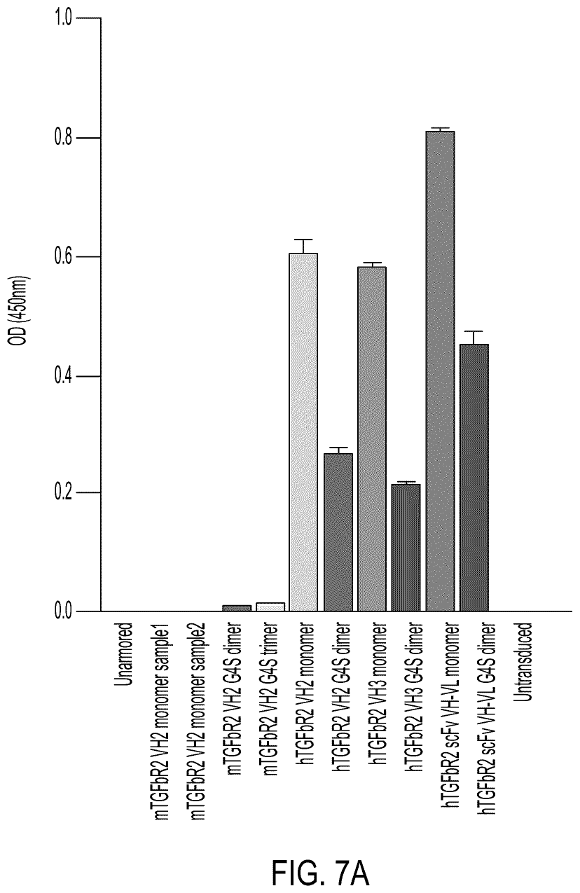

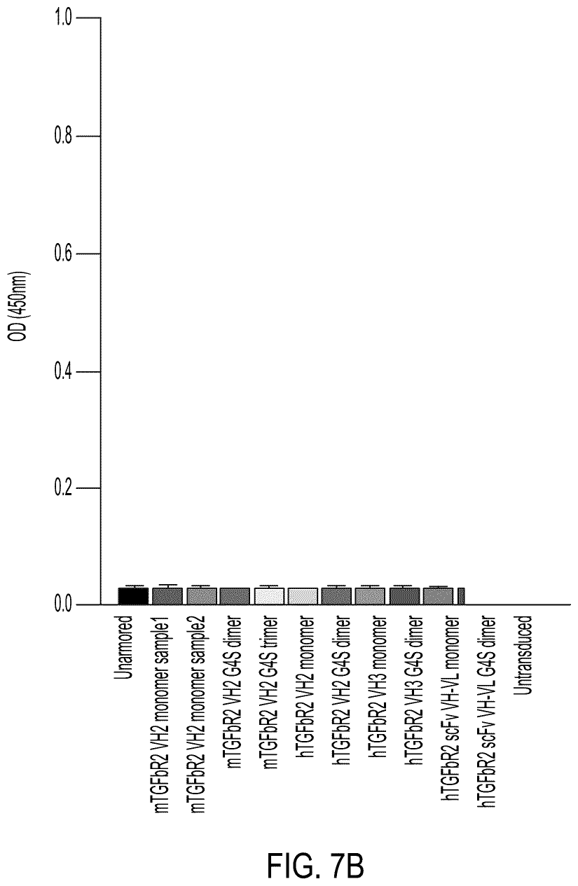

Supernatant was harvested two days after transduction and frozen at −80′C until used for ELISA. ELISA was performed as described in Example 3. Human recombinant TGFbR2-Fc protein was used for capturing binders against human TGFbR2 and mouse recombinant TGFbR2-Fc protein was used for probing binding of TGFbR2 binders to mouse TGFbR2. Binding was detected using anti-flag HRP antibodies and the respective substrate. As shown in FIG. 7, secretion of binders hTGFbR2-VH2 and hTGFbR2-VH3 monomer and dimer and TGFbR2 scFv VH-VL monomer and dimer and their bind to human TGFbR2. None of the binders from the tested supernatants bound to mouse TGFbR2 confirming specificity for human TGFBR2.

### Example 6. In Vivo Anti-Tumor Efficacy of CAR-T Cells Secreting TGF-β Signaling Modulators

This example illustrates in vivo anti-tumor efficacy of anti-TGF-β mAb secreting CAR-T cells. Mouse armored CAR-T cells (co-expressing anti-human CD19 CAR and a TGFb signaling modulator) inhibits growth of syngeneic EMT6-hCD19 tumor better than unarmored CAR-T cells. In addition, armored CAR-T cells reduce liver and lung metastasis. A EMT6 breast carcinoma cell line overexpressing human CD19 as CAR-T target antigen and firefly luciferase was generated. The EMT6 cells were transduced with virus carrying a plasmid encoding human CD19 under the control of an EF1a promoter and puromycin resistance. EMT6-hCD19 cells were positively selected using puromycin and further purified by FACS sorting. EMT6-hCD19 cells were transduced with virus carrying a plasmid encoding firefly luciferase under the control of an EF1a promoter and neomycin resistance (Amsbio) at 5×10⁷ IFU/mL; MOI=10, in the presence of polybrene. EMT6-hCD19-Fluc cells were positively selected using G418 (500 μg/ml).

6-16 week old female Balb/c mice (Jackson Labs) were inoculated with 0.2×10⁶ viable EMT6-hCD19-Fluc tumor cells into the mammary fat pad (orthotopic). 6 days after implantation, the tumor size reached about 50 mm³ and mice were randomized into treatment groups with similar average tumor size (average˜50 mm³) and treated with cyclophosphamide (CPA; 200 mg/kg i.p.). The following day, 500,000 mouse CAR-T cells from congenic CD45.1 Balb/c mice were injected into the tail vein. Group 1 received untransduced T cells, Group 2 received CAR-T cells, and Group 3 received anti-TGF-β scFv VH-VL1 secreting CAR-T cells. Body weights were measured twice weekly to monitor toxicity.

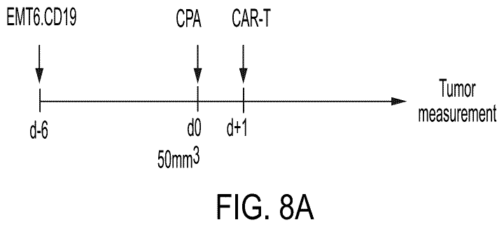

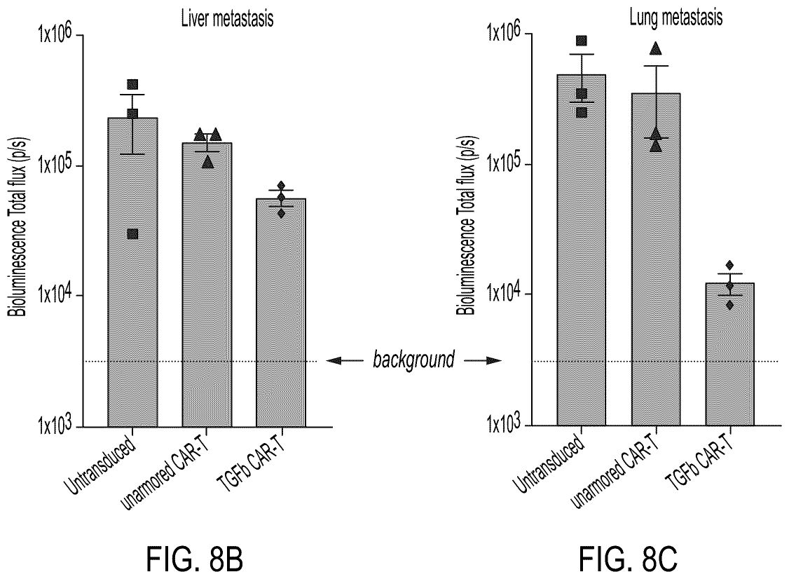

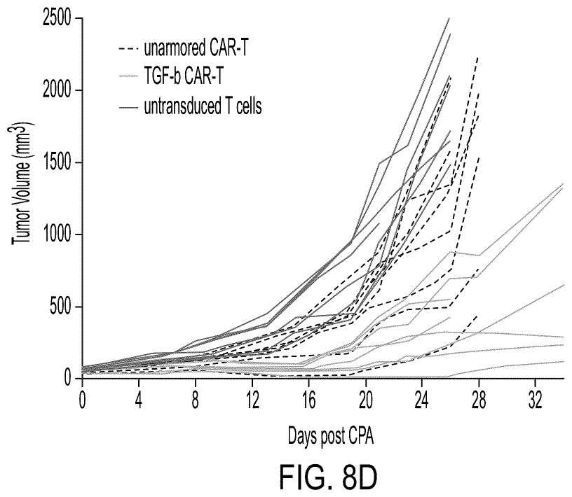

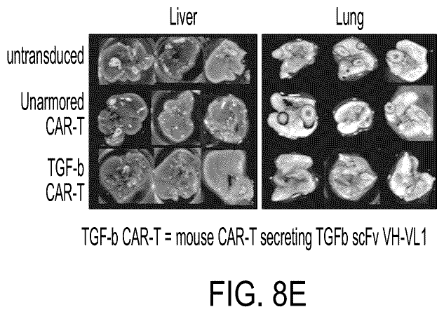

Tumor size was measured twice weekly and tumor volume was calculated using the formula: tumor volume (mm³)=length×width×height×0.5236 (FIG. 8). Any mice with tumors over 2000 mm³ or ulcerating tumors were sacrificed. Anti-tumor efficacy was evaluated as decrease of tumor size as compared to control mice that were injected with untransduced T cells. Complete responders were defined as mice without any detectable tumors.

CAR-T cells secreting TGF-β binder showed high anti-tumor efficacy relative to unarmored CAR-T or untransduced CAR-T cells. Livers and lungs were imaged for firefly luciferase expressing tumor cells by injecting luciferin and imaging by IVIS. D-Luciferin solution (D-Luciferin, Potassium Salt Vivo Glo™ Luciferin) was prepared at 15 mg/ml and was used at 150 mg/kg. Mice treated with CAR-T cells that secrete TGF-β binders were capable of reducing liver and lung metastasis (FIG. 8A-8E).

Mouse CAR-T against human CD19 secreting an inhibitory antibody against TGF-β(TGF-b scFv VH-VL1) inhibit growth of syngeneic EMT6-hCD19 tumor better than unarmored CAR-T cells and reduce liver and lung metastasis. The number of CAR-T cells had previously been titrated to give a suboptimal effect for unarmored CAR-T to identify improved activity of armored CAR-T cells.

### Example 7. Armored Mouse CAR-T Cells that Secrete TGFbR2 Extracellular Domain (ECD) Inhibit TGFb Signaling

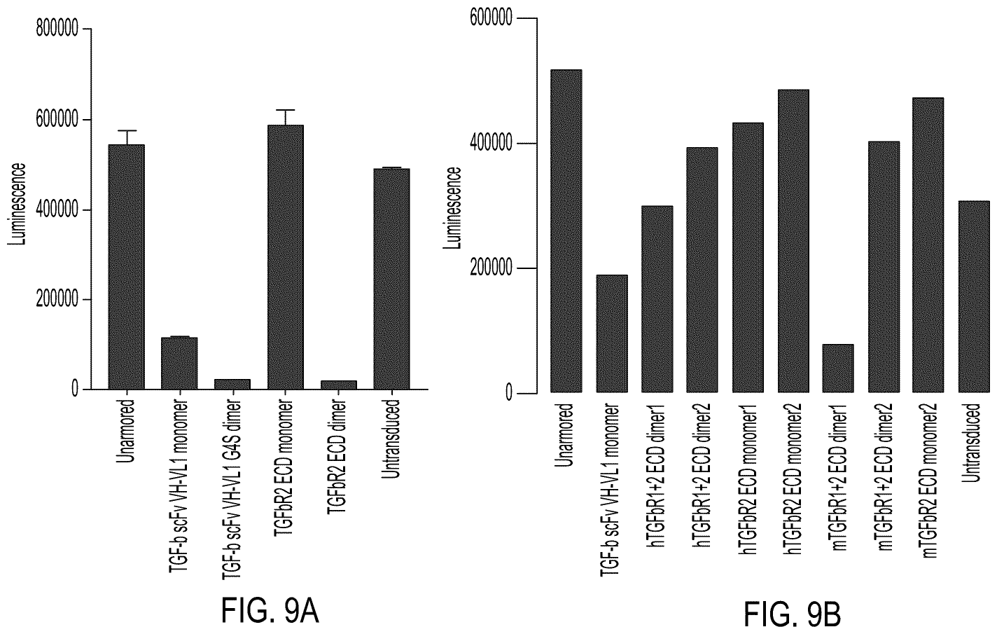

SBE-Luc TGF-β reporter assay was performed comparing supernatant from armored mouse CAR-T secreting different TGF-β ligand traps (TGF-β scFv VH-VL1 to TGFbR2 ECD monomers, homodimers (FIG. 9A) and heterodimers (FIG. 9B)) to unarmored CAR-T. SBE-Luc TGF-β reporter assay showed that supernatant from armored mouse CAR-T against human CD19 that secrete TGFβR2 extracellular domain (ECD) dimer but not monomers inhibits well and shows comparable inhibition to TGF-β scFv VH-VL1 dimer. Supernatants were harvested 2 days after transduction. Inhibition of TGFβR2 ECD heterodimer including a TGFβR2 ECD and TGFβR1 ECD was assessed. identified a TGFβ3R2 ECD heterodimer that inhibits TGF-b signaling more potently than TGF-β scFV VH-VL1. Exemplary TGFβ3R2 ECD sequences are shown in Table 4.

TABLE 4
Exemplary TGFBR2 ECD sequences

TGFbR2 ECD monomer
METDTLLLWVLLLWVPGSTGIPPHVPKSDVEMEAQKDASIHLSCNRTIHPLKHF
NSDVMASDNGGAVKLPQLCKFCDVRLSTCDNQKSCMSNCSITAICEKPHEVCV
AVWRKNDKNITLETVCHDPKLTYHGFTLEDAASPKCVMKEKKRAGETFFMCA
CNMEECNDYIIFSEEYTTSSPDL (SEQ ID NO: 37)
TGFbR2 ECD dimer
METDTLLLWVLLLWVPGSTGIPPHVPKSDVEMEAQKDASIHLSCNRTIHPLKHF
NSDVMASDNGGAVKLPQLCKFCDVRLSTCDNQKSCMSNCSITAICEKPHEVCV
AVWRKNDKNITLETVCHDPKLTYHGFTLEDAASPKCVMKEKKRAGETFFMCA
CNMEECNDYIIFSEEYTTSSPDLGGGGSGGGGSGGGGSGGGGSGGGGSGGGGS
GGGGSGGGGSIPPHVPKSDVEMEAQKDASIHLSCNRTIHPLKHFNSDVMASDN
GGAVKLPQLCKFCDVRLSTCDNQKSCMSNCSITAICEKPHEVCVAVWRKNDKN
ITLETVCHDPKLTYHGFTLEDAASPKCVMKEKKRAGETFFMCACNMEECNDYII
FSEEYTTSSPDL (SEQ ID NO: 39)
mTGFbR2ECD monomer 2
IPPHVPKSDVEMEAQKDASIHLSCNRTIHPLKHFNSDVMASDNGGAVKLPQLCK
FCDVRLSTCDNQKSCMSNCSITAICEKPHEVCVAVWRKNDKNITLETVCHDPKL
TYHGFTLEDAASPKCVMKEKKRAGETFFMCACNMEECNDYIIFSEEYTTSSPDL
(SEQ ID NO: 32)
huTGFbR1 + 2 ECD dimer1
LQCFCHLCTKDNFTCVTDGLCFVSVTETTDKVIHNSMCIAEIDLIPRDRPFVCAP
SSKTGSVTTTYCCNQDHCNKIELPTTVKSSPGLGPVEGGGGSTIPPHVQKSVNN
DMIVTDNNGAVKFPQLCKFCDVRFSTCDNQKSCMSNCSITSICEKPQEVCVAV
WRKNDENITLETVCHDPKLPYHDFILEDAASPKCIMKEKKKPGETFFMCSCSSD
ECNDNIIFSEEYNTSNPD (SEQ ID NO: 33)
huTGFbR1+ 2 ECD dimer2
QCFCHLCTKDNFTCVTDGLCFVSVTETTDKVIHNSMCIAEIDLIPRDRPFVCAPS
SKTGSVTTTYCCNQDHCNGGGGSAVKFPQLCKFCDVRFSTCDNQKSCMSNCSI
TSICEKPQEVCVAVWRKNDENITLETVCHDPKLPYHDFILEDAASPKCIMKEKK
KPGETFFMCSCSSDECNDNIIFSE (SEQ ID NO: 34)
mTGFbR1 + 2 ECD dimer1
LQCFCHLCTKDNFTCETDGLCFVSVTETTDKVIHNSMCIAEIDLIPRDRPFVCAPS
SKTGAVTTTYCCNQDHCNKIELPTTGPFSEKQSAGLGPVELGGGGSIPPHVPKSV
NSDVMASDNGGAVKLPQLCKFCDVRLSTCDNQKSCMSNCSITAICEKPHEVCV
AVWRKNDKNITLETVCHDPKLTYHGFTLEDAASPKCVMKEKKRAGETFFMCA
CNMEECNDYIIFSEEYTTSSPD (SEQ ID NO: 35)
huTGFbR2 ECD monomer 1
LQCFCHLCTKDNFTCVTDGLCFVSVTETTDKVIHNSMCIAEIDLIPRDRPFVCAP
SSKTGSVTTTYCCNQDHCNKIELPTTVKSSPGLGPVE (SEQ ID NO: 40)
huTGFbR2 ECD monomer2
QCFCHLCTKDNFTCETDGLCFVSVTETTDKVIHNSMCIAEIDLIPRDRPFVCAPSS
KTGAVTTTYCCNQDHCNGGGGSAVKLPQLCKFCDVRLSTCDNQKSCMSNCSIT
AICEKPHEVCVAVWRKNDKNITLETVCHDPKLTYHGFTLEDAASPKCVMKE
(SEQ ID NO: 36)

### Example 8. Anti-Tumor Efficacy of Armored CAR-T Cells Secreting a TGF-β Signaling Modulator

This example illustrates the relative in vivo anti-tumor efficacy of anti-TGFβ3 mAb or TGFβ3 R2-ECD secreting CAR-T cells.

Mouse CAR-T secreting TGFβ3R2 ECD1+2 dimer show improved anti-tumor function in vivo as compared to unarmored CAR-T cells. 6-16 week old female Balb/c mice (Jackson Labs) were inoculated with 0.2×10⁶ viable EMT6-hCD19-Fluc tumor cells into the mammary fat pad (orthotopic). 6 days after implantation the tumor size reached about 50 mm3 and mice were randomized into treatment groups with similar average tumor size (average˜50 mm3; n=8 per group) and treated with cyclophosphamide (CPA; 200 mg/kg i.p.). The following day 2 million mouse CAR-T cells from congenic CD45.1 Balb/c mice were injected into the tail vein. Group 1 received untransduced control T cells, Group 2 received unarmored CAR-T cells, Group 3 received TGFbR1+2 ECD dimer secreting CAR-T cells and group 4 received systemic anti-TGF-β antibody (clone 1D11.16.8; 10 mg/kg; 3 injections/week; i.v.). Body weights were measured twice weekly to monitor toxicity. Tumor size was measured twice weekly and tumor volume was calculated using the formula: tumor volume (mm³)=length×width×height×0.5236. Any mice with tumors over 2000 mm³ or ulcerating tumors were sacrificed following the institute's animal health protocol. Anti-tumor efficacy was evaluated as decrease of tumor size as compared to control mice that were injected with untransduced T cells. Complete responders were defined as mice without any detectable tumors.

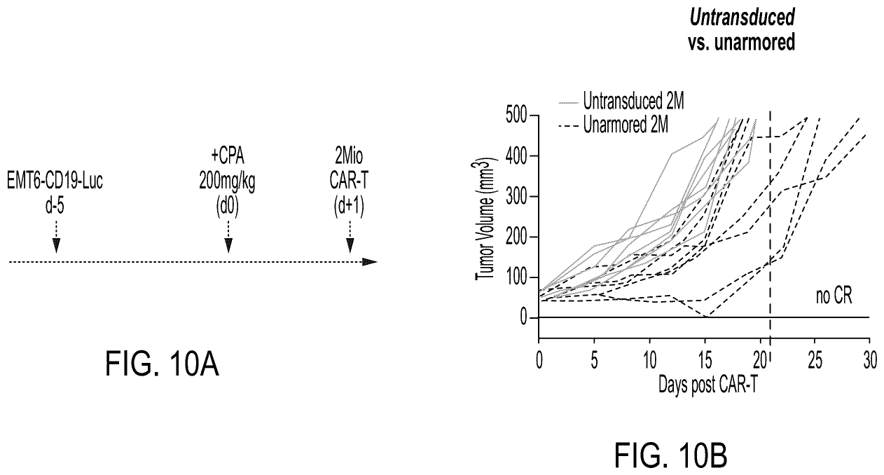

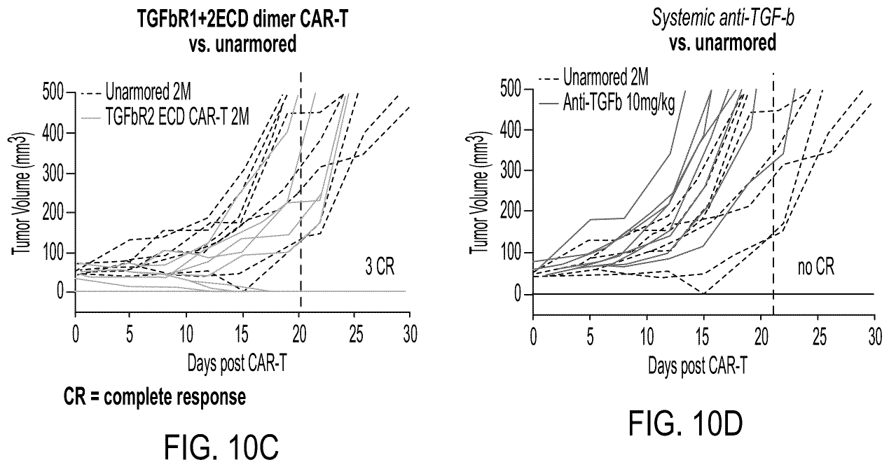

Mouse CAR-T against human CD19 secreting a TGF-β ligand trap (mTGFbR2 ECD1+2 dimer1) inhibit growth of syngeneic EMT6-hCD19 tumor better than unarmored CAR-T cells, inducing 3 complete responses as compared to no complete responses in control mice that received unarmored CAR-T or untransduced T cells or were treated with systemic anti-TGF-β antibody (1D11, 10 mg/kg, 3× per week i.v.). (FIG. 10)

### Example 9. Anti-Tumor Efficacy of Armored CAR-T Cells Secreting a TGF-β Signaling Modulator in Syngeneic Tumor Model (MC38-hCD19)

This example illustrates the relative in vivo anti-tumor efficacy of anti-TGFβ mAb or TGFβ R2-ECD secreting CAR-T cells. Improved function of mouse CAR-T cells armored with anti-TGF-b mAb (TGF-b scFv VH-VL1) a different syngeneic tumor model (MC38-hCD19).

An MC38 colorectal cancer cell line overexpressing human CD19 as CAR-T target antigen and Firefly luciferase was generated and used for imaging. In brief, MC38 cells were transduced with virus carrying a plasmid encoding for human CD19 under control of an EF1a promoter and puromycin resistance (CD19_FL_WT_pLVX-EF1a-IRES-Puro). MC38-hCD19 cells were positively selected using puromycin. MC38-hCD19 cells were transduced with virus carrying a plasmid encoding for firefly luciferase under control of an EF1a promoter and neomycin resistance (Amsbio, CAT #LVP435-PBS, at 5×10\hat{ }7 IFU/mL; MOI=10) in the presence of polybrene. MC38-hCD19-Fluc cells were positively selected using G418 (geneticin).

6-16 week old female C57BL/6 mice (Jackson Labs) were inoculated with 0.2×10⁶ viable MC38-hCD19-Fluc tumor cells s.c. Seven days after implantation the tumor size reached about 50 mm3 and mice were randomized into treatment groups with similar average tumor size (average˜50 mm3; n=8 per group) and treated with cyclophosphamide (CPA; 200 mg/kg i.p.). The following day 100,000 mouse CAR-T cells (or untransduced T cells as negative control) from congenic CD45.1 C57BL/6 mice were injected into the tail vein. Group 1 received untransduced T cells. Group 2 received unarmored CAR-T cells. Group 3 received anti-TGF-β secreting CAR-T cells (TGF-b scFv VH-VL1). Body weights were measured twice weekly to monitor toxicity. Tumor size was measured twice weekly and tumor volume was calculated using the formula: tumor volume (mm³)=length×width×height×0.5236. Any mice with tumors over 2000 mm³ or ulcerating tumors were sacrificed following the institute's animal health protocol. Anti-tumor efficacy was evaluated as decrease of tumor size as compared to control mice that were injected with untransduced T cells. Complete responders were defined as mice without any detectable tumors.

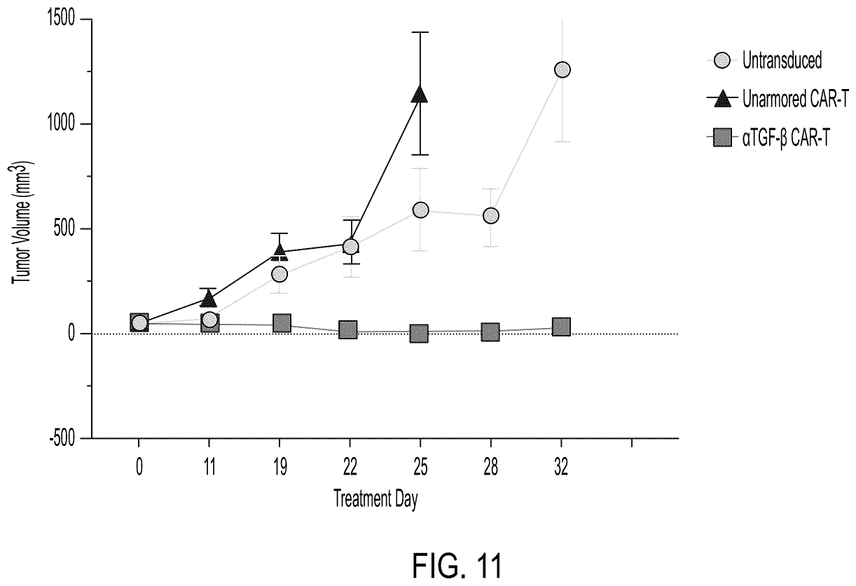

CAR-T cells secreting an inhibitory binder against TGF-β (TGF-b scFv VH-VL1) showed superior efficacy to unarmored CAR-T, inducing 7 complete responses among 8 treated mice as compared to no complete responses in the control groups that received an equal amount of either unarmored CAR-T or untransduced T cells. (FIG. 11)

### Example 10. CAR-T Cells Secreting a TGF-β Signaling Modulator Enhance Activation of the Host Immune Response

This example demonstrates RNA Seq showed enhanced activation of the host immune response by CAR-T cells secreting a binder against TGF-β.

6-16 week old female Balb/c mice (Jackson Labs) were inoculated with 0.2×10⁶ viable EMT6-hCD19-Fluc tumor cells into the mammary fat pad (orthotopic). 6 days after implantation the tumor size reached about 50 mm3 and mice were randomized into treatment groups with similar average tumor size (average˜50 mm3; n=8 per group) and treated with cyclophosphamide (CPA; 200 mg/kg i.p.). The following day 2 million mouse CAR-T cells from congenic CD45.1 Balb/c mice were injected into the tail vein. Group 1 received untransduced control T cells, Group 2 received unarmored CAR-T cells, Group 3 received anti-TGF-βscFv VH-VL1 secreting CAR-T cells. Group 4 was treated with systemic anti-TGF-β antibody (clone 1D11.16.8; BioXcell; 10 mg/kg; 3× per week 1.v.) and group 5 received isotype control antibody (clone MOPC21; BioXcell; 10 mg/kg; 3× per week 1.v.). Body weights were measured twice weekly to monitor toxicity. Tumor size was measured twice weekly and tumor volume was calculated using the formula: tumor volume (mm³)=length×width×height×0.5236.

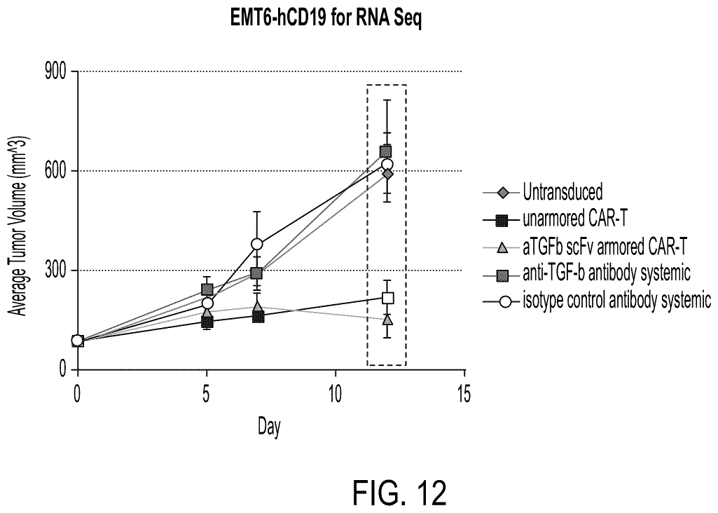

Mice were euthanized on day+12 and tumors were harvested, snap frozen and kept at −80′C. RNA was extracted and RNA-Seq followed by computational analysis was performed as shown in FIG. 12.

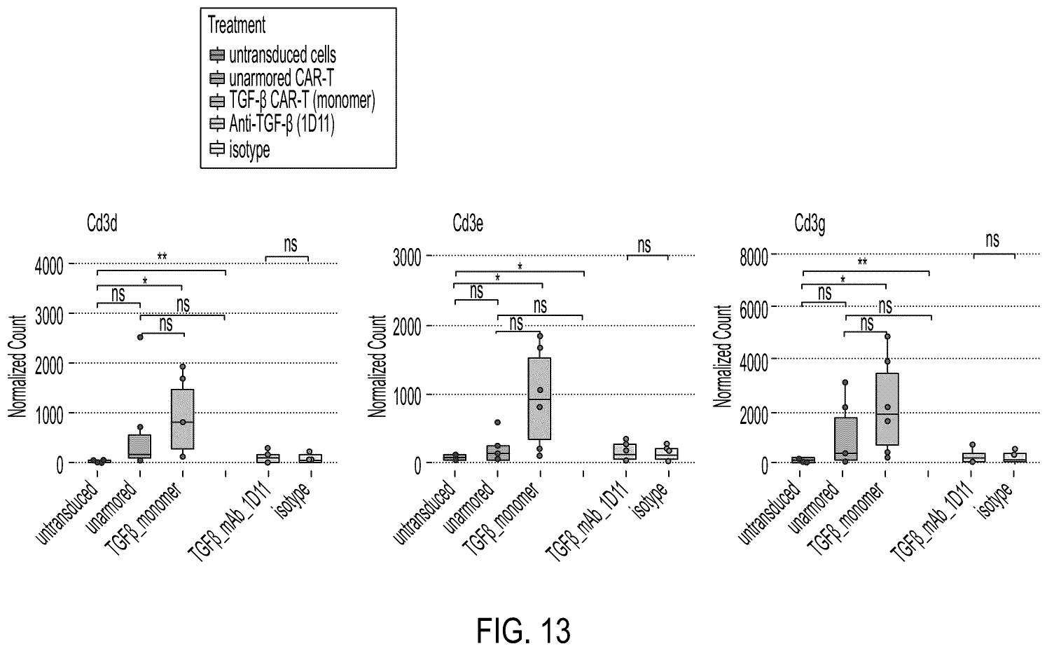

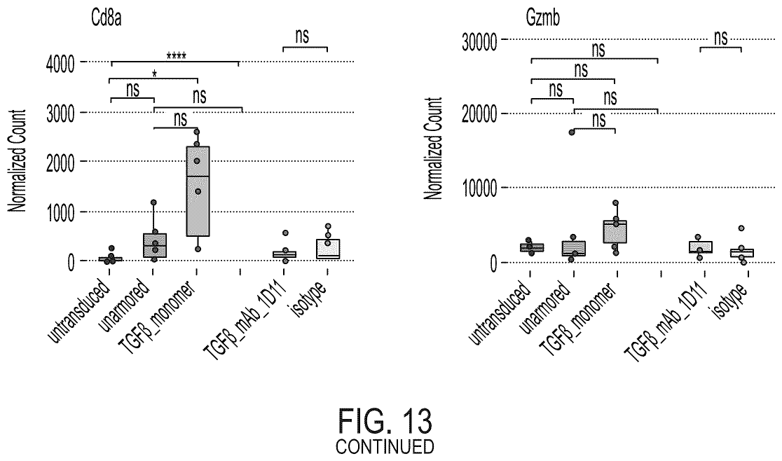

Tumors from mice that received TGF-βscFv VH-VL1 secreting CAR-T had significantly increased scores for tumor infiltrating T cells (CD3d+, CD3e+, CD3g+) and in particular CD8+ T cells (CD8a+) and cytotoxic T cells (GzmB+) as compared to mice from the other groups (FIG. 13)

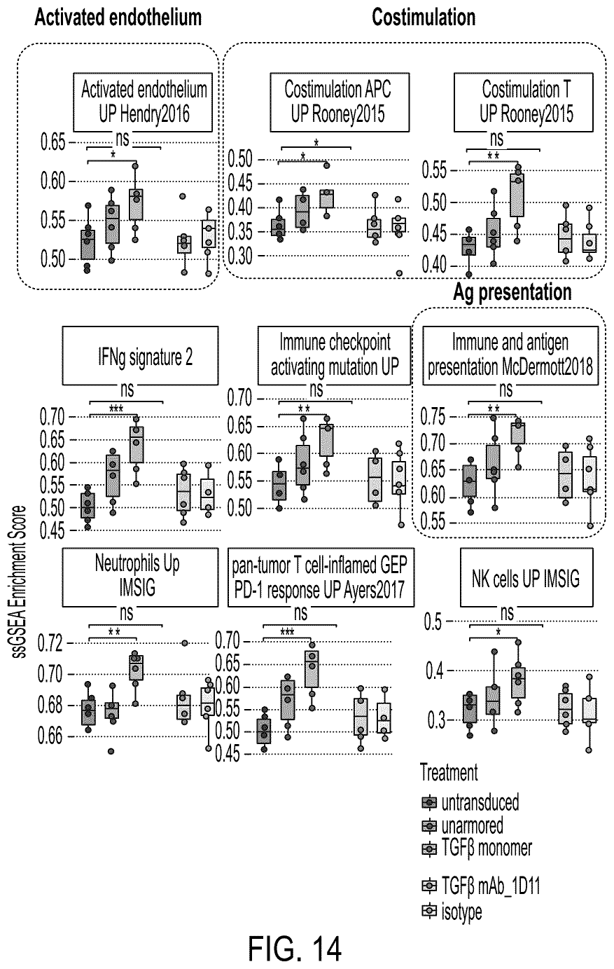

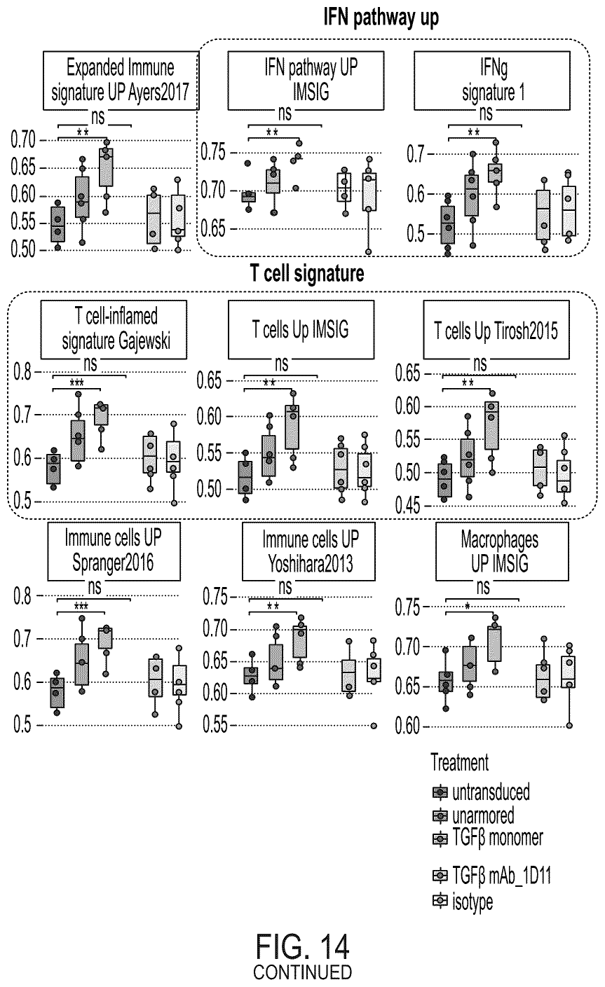

ssGSEA enrichment scores showed increase of T cell signatures and IFNg signatures in tumors from mice that received TGF-βcFv VH-VL1 secreting CAR-T indicating increased infiltration of CAR-T cells and/or activation of the endogenous immune system. The increased signatures for activated endothelium, costimulation and antigen presentation in tumors from mice that received TGF-βscFv VH-VL1 secreting CAR-T clearly shows activation of the endogenous immune system. Hence, armoring of CAR-T cells with blocking antibodies (or other binders) inhibiting the TGF-β pathway anti-tumor efficacy, at least partially by improving the endogenous immune response. (FIG. 14)

### Example 11. FACS of Tumor Samples from EMT6-hCD19 Mice Treated with Unarmored CAR-T Cells

6-16 week old female Balb/c mice (Jackson Labs) were inoculated with 0.2×10⁶ viable EMT6-hCD19-Fluc tumor cells into the mammary fat pad (orthotopic). 6 days after implantation the tumor size reached about 50 mm3 and mice were randomized into treatment groups with similar average tumor size (average˜50 mm3; n=8 per group) and treated with cyclophosphamide (CPA; 200 mg/kg i.p.). The following day 2 million mouse CAR-T cells from congenic CD45.1 Balb/c mice were injected into the tail vein. Group 1 received untransduced control T cells, Group 2 received unarmored CAR-T cells, Group 3 received anti-TGF-b scFv VH-VL1 monomer secreting CAR-T cells. Body weights were measured twice weekly to monitor toxicity. Tumor size was measured twice weekly and tumor volume was calculated using the formula: tumor volume (mm³)=length×width×height×0.5236.

Mice were euthanized on day+7 and tumors were harvested, weighed and processed for FACS analysis. In brief, tumors were cut into small pieces and digested using Mouse Tumor Dissociation Kits (Miltenyi) according to manufacturer's instructions. The samples were resuspended in PBS 2% FCS, filtered and plated into a 96 well plat for FACS staining. Fc receptors were blocked (TruStain FcX (anti-mouse CD16/32) Antibody; Biolegend) and the CAR was labelled for 1 hr at 4′C using rhuCD19 (RnD Systems) and thereafter labelled for hCD19-Fc using anti-human IgG Fc antibody, surface markers including TCRa/b, CD8a, CD4, CD25, CD62L, CD11b, Gr1, CD11c, CD45.1 and CD45, live cells were stained using fixable viability dye (ebioscience) and intracellular antigens including GzmB, Ki67 and FoxP3 were stained using eBioscience Foxp3/Transcription Factor Staining Buffer Set (Thermofisher). Samples were filtered and acquired on a flow cytometer BD Fortessa.

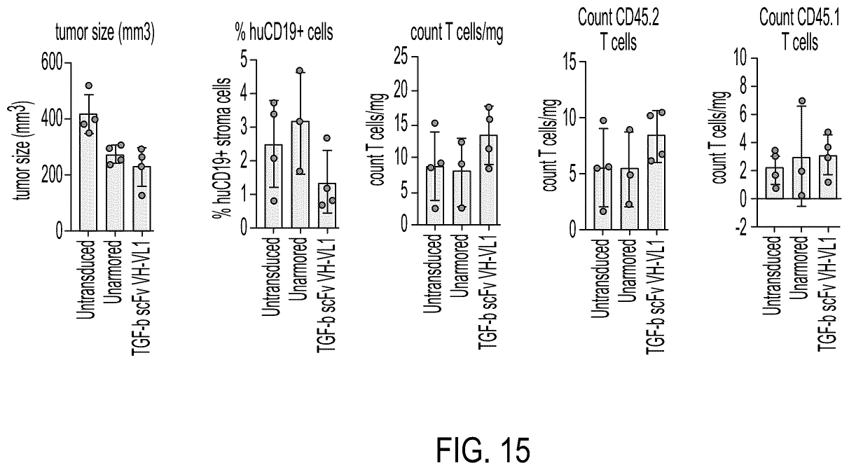

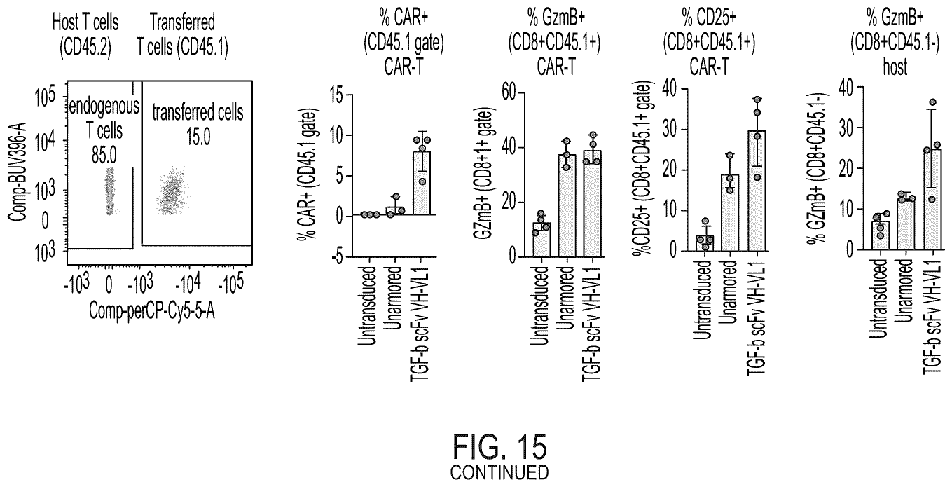

As shown in FIG. 15, FACS staining showed a decrease of hCD19+ tumor cells in samples from mice that received CAR-T secreting TGF-β scFv VH-VL as compared to controls that received untransduced cells or unarmored CAR-T and an increase in T cell infiltration (per mg tumor tissue). Gating on CD45.1+ and CD45.1− T cells shows in particular an increase in endogenous T cell infiltration (CD45.1−). transferred CAR-T cells (CD45.1+) from these samples had higher CAR expression level and CD8+ T cells showed higher CD25 expression indicating increase activation. CD8+ T cells from host T cells (CD45.1−) had higher expression of GzmB indicating higher cytotoxicity. In summary, these FACS data indicates that armoring of CAR-T cells with binders against TGF-βenhances function of CAR-T cells and the endogenous immune response.

### Example 12. Xenograft Model Shows Improved Function of Human GCC-CAR-T Cells Armored with Anti-TGF-3 or Anti-TGFβR2 Blocking Antibodies

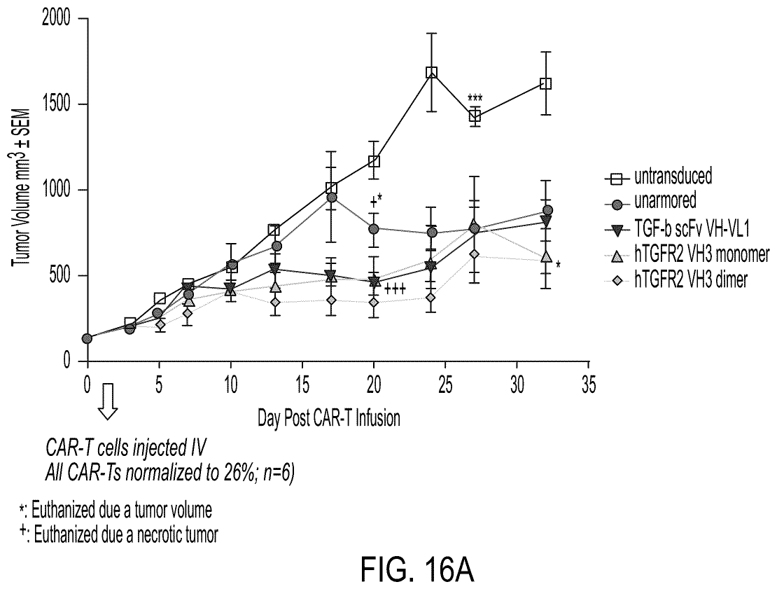

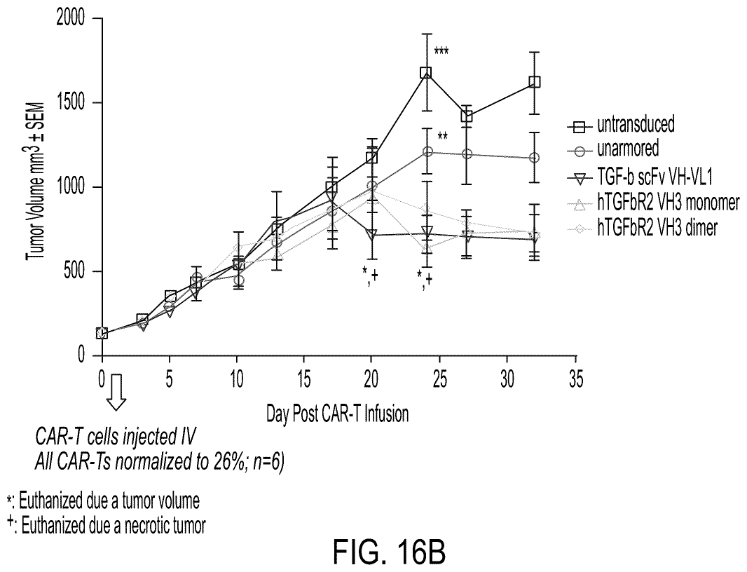

6-16 week old female NSG mice (Jackson Labs) were inoculated subcutaneously with 2×10⁶ viable GSU tumor cells. 7 days after implantation the tumor size reached about 50 mm3 and mice were randomized into treatment groups with similar average tumor size (average˜50 mm3; n=6 per group). The following day 500,000 or 100,000 human GCC CAR-T cells were injected into the tail vein. Group 1 received untransduced control T cells, Group 2 received unarmored CAR-T cells, Group 3 received anti-TGF-b scFv VH-VL1 monomer secreting CAR-T cells, Group 4 received anti-TGFβR2 VH3 monomer and Group 5 anti-TGFβR2 VHH dimer secreting CAR T cells. Body weights were measured twice weekly to monitor toxicity. Tumor size was measured twice weekly and tumor volume was calculated using the formula: tumor volume (mm³)=length×width×height×0.5236. GCC-CAR-T cells armored with anti-TGF-β or anti-TGFβR2 blocking antibodies showed faster response than unarmored control CAR-T at 500,000 transferred cells and improved anti-tumor efficacy at 100,000 transferred CAR-T cells. (FIG. 16).

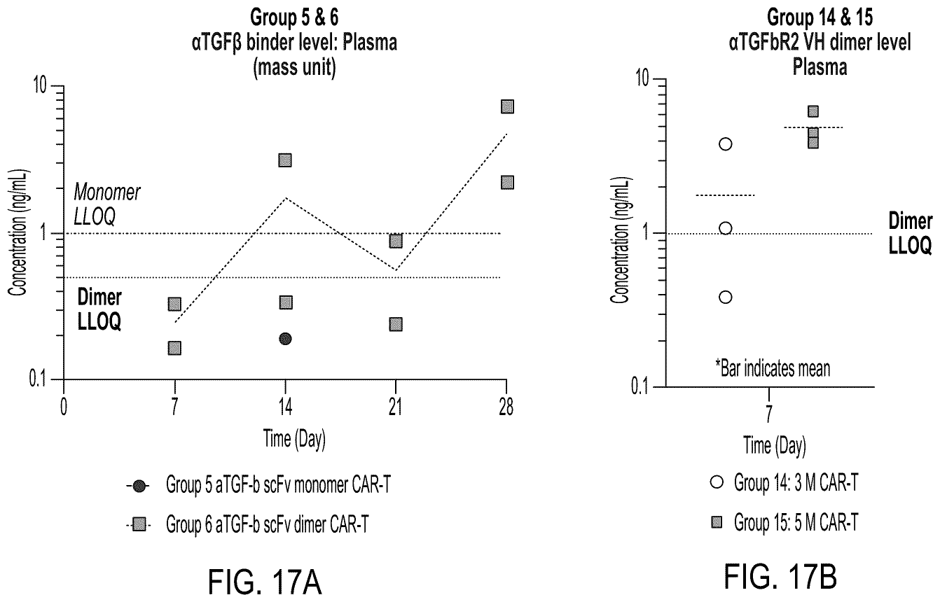

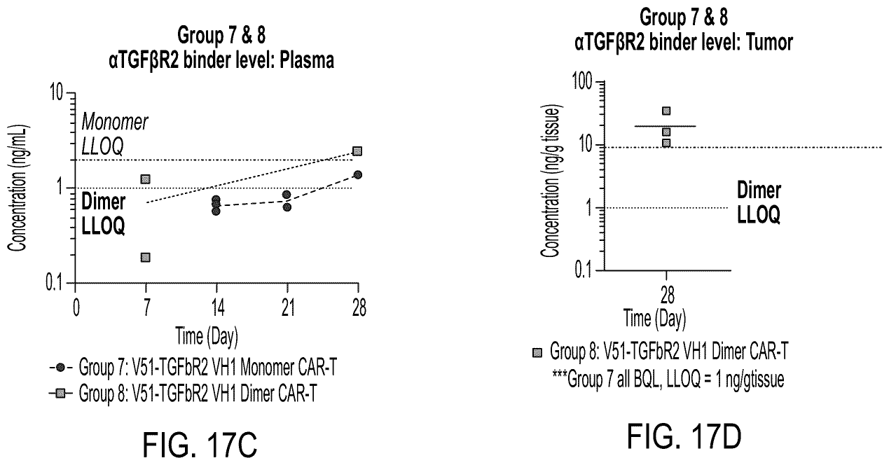

Tumor and plasma concentrations of TGFβ modulators were determined using an anti-Flag immune capture LC/MS assay. As shown in FIG. 17A-17D, Low quantities of the secreted TGF-β antibody or anti-TGFbR2 antibody in the circulation of mice treated with armored CAR-T cells. Plasma was collected from mice treated with the indicated amount of armored or unarmored anti-GCC CAR-T cells using EDTA tubes.

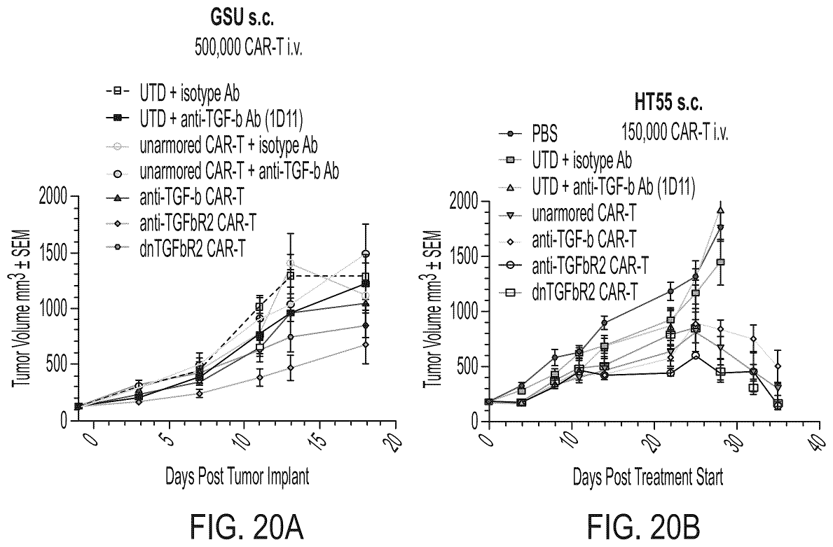

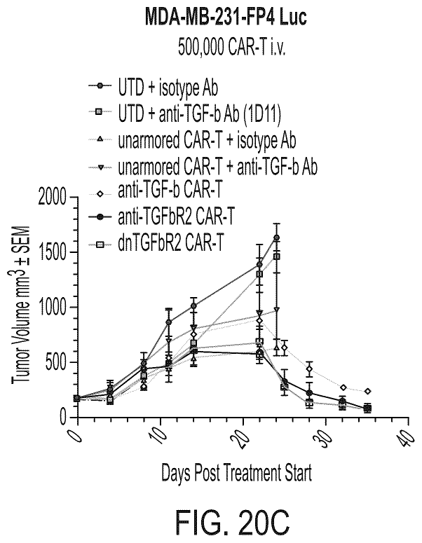

As shown in FIG. 20A-20C, armored CAR-T cells also demonstrated anti-tumor activity in GCC positive GSU, HT55 and MDA-MB-231-FP4 Luc xenograft models.

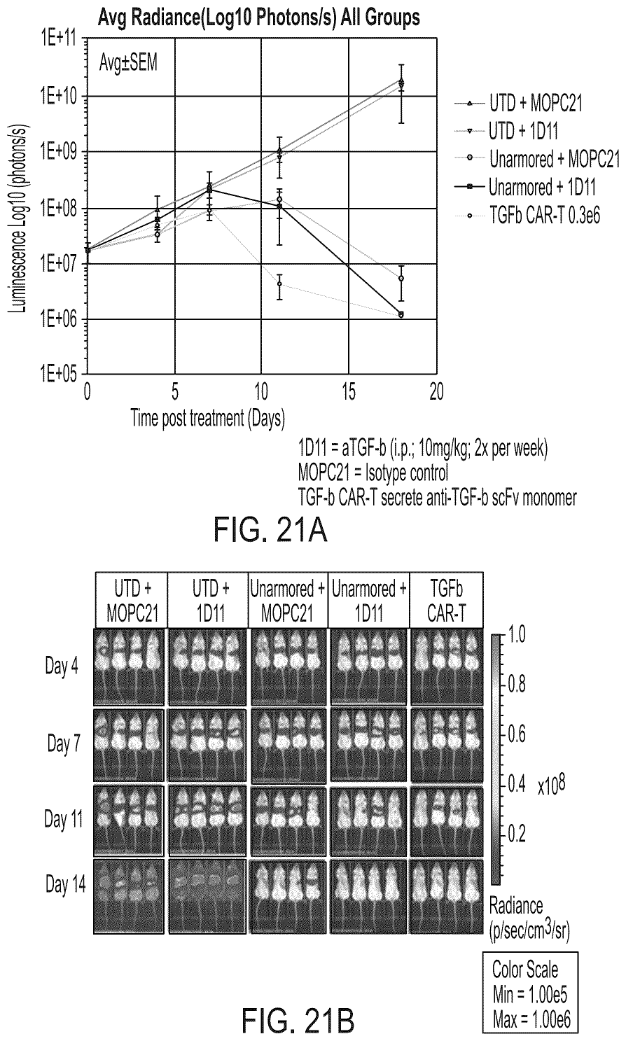

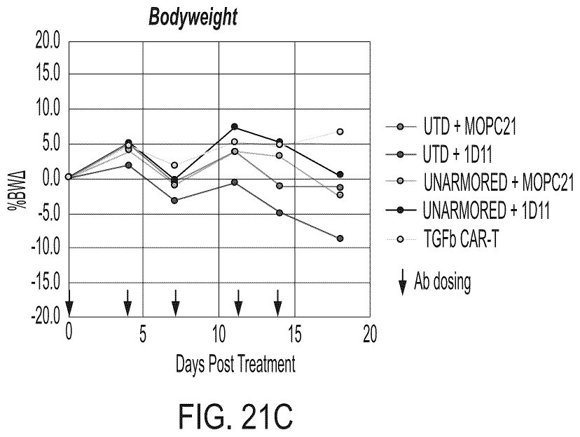

A liver metastasis was evaluated using intrasplenic injection of HT55 tumor cells followed by intravenous injection of CAR-T cells. Armored CAR-T cells slowed metastasis to the liver relative to isotype control (FIG. 21A-21C)

### Example 13. Repeat Antigen Stimulation in GCC Positive Tumors

100,000 anti-GCC CAR-T cells unarmored or armored (co-expressing an anti-GCC CAR and a TGFβ modulator (e.g., TGFβR2-VHH)) were co-cultured in duplicates with 200,000 HT29-GCC or HT29 parental (GCC negative) tumor cells in the presence or absence of TGF-β (1 ng/ml or 10 ng/ml). Every 3-4 days half of the CAR-T cells per well were transferred to a new plate of tumor cells under the same conditions (with or without TGF-β 1 ng/ml or 10 ng/ml). Supernatants were harvested and frozen for later evaluation. Cells were evaluated for cell counting, and FACS staining analysis

Tumor cells were assessed using CellTiterGlo (Promega) according to manufacturer's protocol. The plates were analyzed using a Pherastar plate reader. Percent killing was assessed using the following formula:

% killing=(1−(signal from tested well/signal from control wells))*100

The control wells contained tumor cells co-cultured with untransduced T cells from the same donor as used for the CAR-T cells. FACS staining was performed once a week using fluorochrome conjugated antibodies against human CD4, CD8, CD25 and the exhaustion markers PD-1, TIM-3, Lag-3 and TIGIT antibodies (Biolegend). Dead cells were excluded using fixable viability dye efluor 506 (Thermofisher; according to manufacturer's protocol). CAR expressing cells were incubated with GCC-hFc for 1 hour at 4′C, washed with PBS 2% FCS and detected with a secondary mouse anti-human IgG antibody (30 minutes, 4′C).

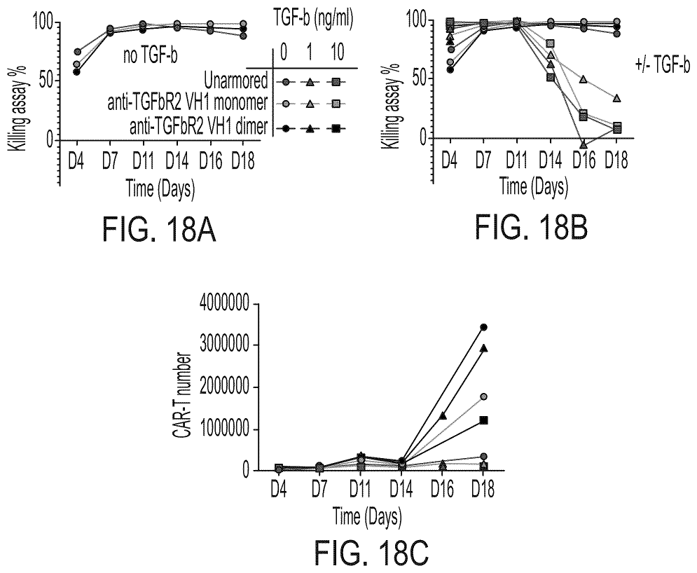

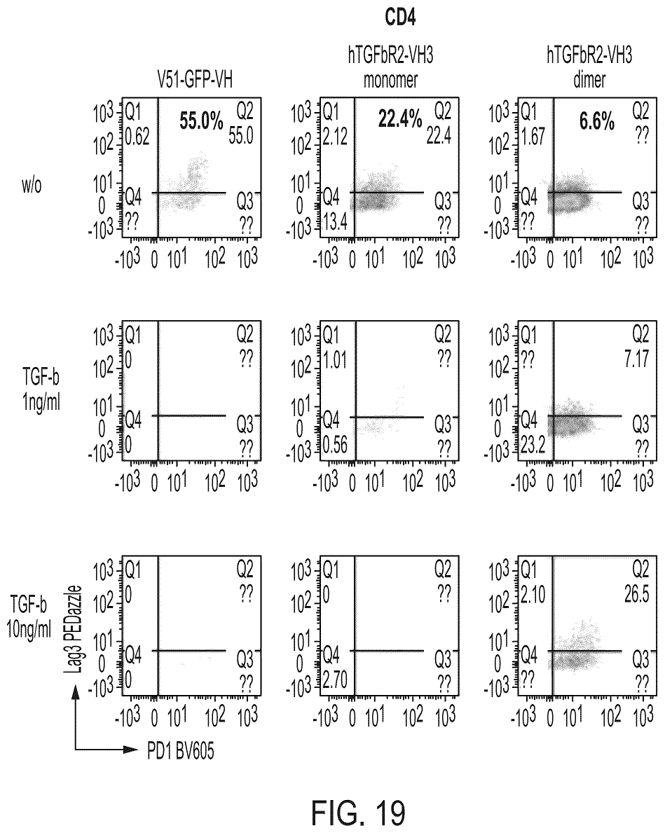

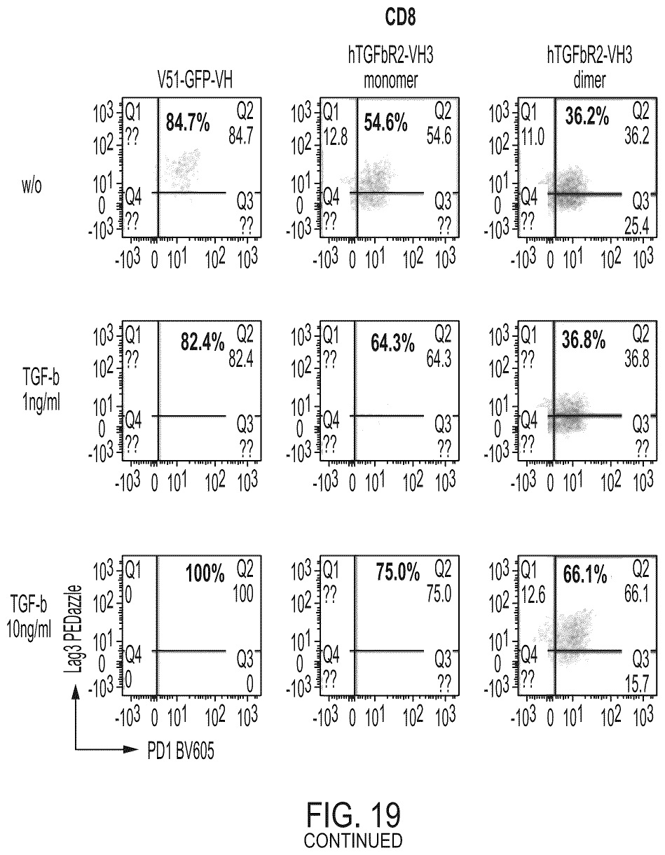

After several rounds of restimulation with target cells, simulating chronic antigen activation, TGF-β induces inhibition of CAR-T cell function. Only CAR-T cells secreting the TGFβmodulator (e.g., TGFβR2 VHH dimer) are protected from the inhibitory effects of TGF-β (1 ng/ml or 10 ng/ml) stimulation (FIG. 18A-18C). The inhibitory effect on CAR-T killing correlates with inhibition of proliferation and induction of the exhaustion marker Lag3.

### Example 14. Repeat Antigen Stimulation in Mesothelin (Msln) Positive Tumors

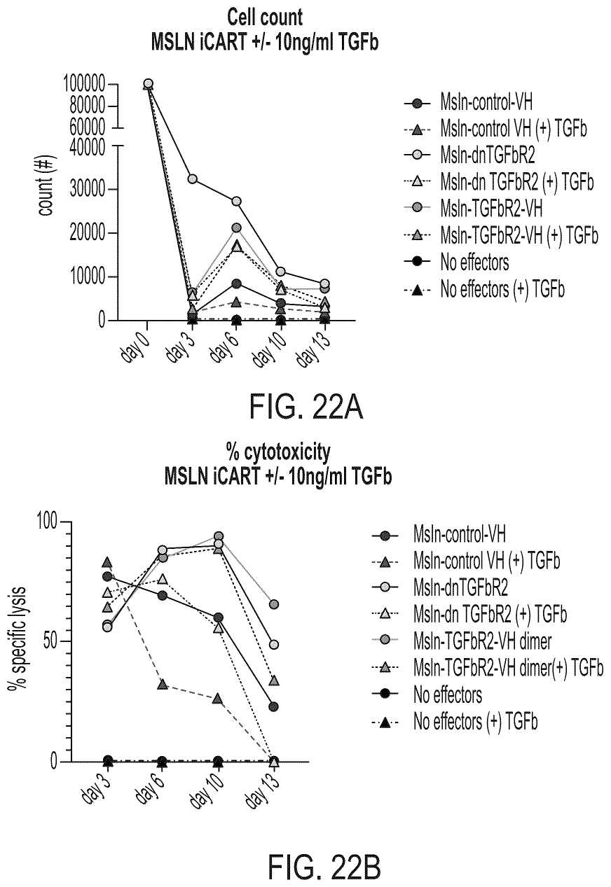

Approximately 100,000 iPSC derived anti-Msln CAR-T cells, co-expressing a CAR against Msln together with a TGFβ modulator (e.g., TGFβR2-VH or dnTGFbR2) or a control VH against GFP (Msln-control VH) were co-cultured in duplicates with 40,000 MiaPaca-2 tumor cells overexpressing human Msln, in the presence or absence of TGF-β (R&D Systems, 10 ng/ml). The TGFβR2-VH was secreted from the CAR-T cell while the dnTGFbR2 was bounded to the membrane of the CAR-T cell. Every 3-4 days, half of the CAR-T cells per well were transferred to a new plate of tumor cells under the same conditions (with or without TGF-β 10 ng/ml). Supernatants were harvested and frozen for later evaluation. CAR-T cells were counted by flow cytometry and FACS phenotyping was performed at selected time-points (FIG. 22A).

Viability of tumor cells was assessed using CellTiterGlo (Promega) according to manufacturer's protocol. The plates were analyzed using a Pherastar plate reader. Percent killing was assessed using the following formula:

$$\% killing = 1 - ⌈ \frac{( signal from tested well )}{( signal from control wells )} ⌉ \times 100$$

The control wells contained tumor cells alone without effector (i.e. CAR-T) cells. The percent cytotoxicity is shown in FIG. 22B.

Cell counts were performed by excluding dead cells using Sytox Red dye (Thermofisher, according to manufacturer's protocol) and equal volumes of cell suspension were acquired on a Fortessa flow cytometer (BD Biosciences) using an HTS unit. Live CAR-T cells were counted by gating on live cells, single cells and size. Results were extrapolated to obtain cell numbers per well.

It was observed that after several rounds of restimulation with target cells, simulating chronic antigen activation, TGF-β induced inhibition of CAR-T cell function (i.e. killing) and inhibited proliferation of CAR-T cells. Only CAR-T cells expressing the TGFβ modulator (e.g., secretion of TGFβR2 VH dimer or expression of membrane bound dnTGFbR2) but not a control VH were protected from the inhibitory effects of TGF-β (10 ng/ml).

### Table of Sequences

Table 5 below provides descriptions and sequences disclosed herein.

TABLE 5
Table of Sequences

| Description/ | |
|---|---|
| SEQ ID NO | Sequence |
| SEQ ID NO: 1 | QVQLVQSGAEVKKPGSSVKVSCKASGYTFSSNVISWVRQAPGQG |
| TGFb scFv VH- | LEWMGGVIPIVDIANYAQRFKGRVTITADESTSTTYMELSSLRSE |
| VL1 | DTAVYYCASTLGLVLDAMDYWGQGTLVTVSSGGGGSGGGGSG |
| | GGGSALETVLTQSPGTLSLSPGERATLSCRASQSLGSSYLAWYQQ |
| | KPGQAPRLLIYGASSRAPGIPDRFSGSGSGTDFTLTISRLEPEDFAV |
| | YYCQQYADSPITFGQGTRLEIKR |
| SEQ ID NO: 2 | QVQLVQSGAEVKKPGSSVKVSCKASGYTFSSNVISWVRQAPGQG |
| TGFb scFv VH- | LEWMGGVIPIVDIANYAQRFKGRVTITADESTSTTYMELSSLRSE |
| VL2 | DTAVYYCASTLGLVLDAMDYWGQGTLVTVSSGGGGSGGGGSG |
| | GGGSETVLTQSPGTLSLSPGERATLSCRASQSLGSSYLAWYQQKP |
| | GQAPRLLIYGASSRAPGIPDRFSGSGSGTDFTLTISRLEPEDFAVYY |
| | CQQYADSPITFGQGTRLEIKR |
| SEQ ID NO: 3 | ETVLTQSPGTLSLSPGERATLSCRASQSLGSSYLAWYQQKPGQAP |
| TGFb scFv VL- | RLLIYGASSRAPGIPDRFSGSGSGTDFTLTISRLEPEDFAVYYCQQ |
| VH | YADSPITFGQGTRLEIKRGGGGSGGGGSGGGGSQVQLVQSGAEV |
| | KKPGSSVKVSCKASGYTFSSNVISWVRQAPGQGLEWMGGVIPIV |
| | DIANYAQRFKGRVTITADESTSTTYMELSSLRSEDTAVYYCASTL |
| | GLVLDAMDYWGQGTLVTVSS |
| SEQ ID NO: 4 | QLQVQESGPGLVKPSETLSLTCTVSGGSISNSYFSWGWIRQPPGK |
| TGFbR2 scFv | GLEWIGSFYYGEKTYYNPSLKSRATISIDTSKSQFSLKLSSVTAAD |
| VH-VL | TAVYYCPRGPTMIRGVIDSWGQGTLVTVSSGGGGSGGGGSGGG |
| | GSEIVLTQSPATLSLSPGERATLSCRASQSVRSYLAWYQQKPGQA |
| | PRLLIYDASNRATGIPARFSGSGSGTDFTLTISSLEPEDFAVYYCQQ |
| | RSNWPPTFGQGTKVEIK |
| SEQ ID NO: 5 | EIVLTQSPATLSLSPGERATLSCRASQSVRSYLAWYQQKPGQAPR |
| TGFbR2 scFv | LLIYDASNRATGIPARFSGSGSGTDFTLTISSLEPEDFAVYYCQQRS |
| VL-VH | NWPPTFGQGTKVEIKGGGGSGGGGSGGGGSQLQVQESGPGLVKP |
| | SETLSLTCTVSGGSISNSYFSWGWIRQPPGKGLEWIGSFYYGEKT |
| | YYNPSLKSRATISIDTSKSQFSLKLSSVTAADTAVYYCPRGPTMIR |
| | GVIDSWGQGTLVTVSS |
| SEQ ID NO: 6 | EVQLLESGGGLVQPGGSLRLSCAASGFTFTTYGMGWVRQAPGK |
| mTGFbR2 VH1 | GLEWVSWIEKTGNKTYYADSVKGRFTISRDNSKNTLYLQMNSLR |
| | AEDTAVYYCAKARHIKVRSRDFDYWGQGTLVTVSS |
| SEQ ID NO: 7 | EVQLLESGGGLVQPGGSLRLSCAASGFTFTTYGMGWVRQAPGK |
| | GLEWVSWIEKTGNKTYYADSVKGRFTISRDNSKNTLYLQMNSLR |
| | AEDTAVYYCAKAGRHIKVRSRDFDYWGQGTLVTVSS |
| SEQ ID NO: 8 | EVQLLESGGGLVQPGGSLRLSCAASGFTFGTEQMWWVRQAPGK |
| hTGFbR2 VH1 | GLEFVSRIDSPGGRTYYADSVKGRFTISRD |
| | NSKNTLYLQMNSLRAEDTAVYYCAKRRPTGVSGTFYDYWGQGT |
| | LVTVSS |
| SEQ ID NO: 9 | EVQLLESGGGLVQPGGSLRLSCAASGFTFGTEQMWWVRQAPGK |
| TGFbR2 VHH | GLEFVSRIDSPGGRTYYADSVKGRFTISRDNSKNTLYLQMNSLRA |
| dimer | EDTAVYYCAKRRPTGVSGTFYDYWGQGTLVTVSSggggsEVQLLE |
| | SGGGLVQPGGSLRLSCAASGFTFGTEQMWWVRQAPGKGLEFVS |
| | RIDSPGGRTYYADSVKGRFTISRDNSKNTLYLQMNSLRAEDTAV |
| | YYCAKRRPTGVSGTFYDYWGQGTLVTVSS |
| SEQ ID NO: 10 | EVQLLESGGGLVQPGGSLRLSCAASGFTFGTEQMWWVRQAPGK |
| | GLEFVSRIDSPGGRTYYADSVKGRFTISRDNSKNTLYLQMNSLRA |
| | EDTAVYYCAKRRPTGVSGTFYDYWGQGTLVTVSSggggsggggsEV |
| | QLLESGGGLVQPGGSLRLSCAASGFTFGTEQMWWVRQAPGKGL |
| | EFVSRIDSPGGRTYYADSVKGRFTISRDNSKNTLYLQMNSLRAED |
| | TAVYYCAKRRPTGVSGTFYDYWGQGTLVTVSS |
| SEQ ID NO: 11 | EVQLLESGGGLVQPGGSLRLSCAASGFTFGTEQMWWVRQAPGK |
| | GLEFVSRIDSPGGRTYYADSVKGRFTISRDNSKNTLYLQMNSLRA |
| | EDTAVYYCAKRRPTGVSGTFYDYWGQGTLVTVSSGGSEPKSsDK |
| | THTCPPCgggssgggsgGQPREPQVYTLPPSRDELTKNQVSLTCLVKG |
| | FYPSDIAVEWESNGQPENNYKTTPPVLDSDGSFFLYSKLTVDKSR |
| | WQQGNVFSCSVMHEALHNHYTQKSLSLSPG |
| SEQ ID NO: 12 | EVQLLESGGGLVQPGGSLRLSCAASGFTFGTEQMWWVRQAPGK |
| | GLEFVSRIDSPGGRTYYADSVKGRFTISRDNSKNTLYLQMNSLRA |
| | EDTAVYYCAKRRPTGVSGTFYDYWGQGTLVTVSSGGSEPKSsDK |
| | THTCPPCgggssgggsgGQPREPQVYTLPPSRDELTKNQVSLTCLVKG |
| | FYPSDIAVEWESNGQPENNYKTTPPVLDSDGSFFLYSKLTVDKSR |
| | WQQGNVFSCSVMHEALHNHYTQKSLSLSPGggggsggsEVQLLESG |
| | GGLVQPGGSLRLSCAASGFTFGTEQMWWVRQAPGKGLEFVSRID |
| | SPGGRTYYADSVKGRFTISRDNSKNTLYLQMNSLRAEDTAVYYC |
| | AKRRPTGVSGTFYDYWGQGTLVTVSS |
| SEQ ID NO: 13 | EVQLLESGGGLVQPGGSLRLSCAASGFTFGTEQMWWVRQAPGK |
| | GLEFVSRIDSPGGRTYYADSVKGRFTISRDNSKNTLYLQMNSLRA |
| | EDTAVYYCAKRRPTGVSGTFYDYWGQGTLVTVSSgggssgggsgGQ |
| | PREPQVYTLPPSRDELTKNQVSLTCLVKGFYPSDIAVEWESNGQP |
| | ENNYKTTPPVLDSDGSFFLYSKLTVDKSRWQQGNVFSCSVMHEA |
| | LHNHYTQKSLSLSPG |
| SEQ ID NO: 14 | EVQLLESGGGLVQPGGSLRLSCAASGFTFGTEQMWWVRQAPGK |
| | GLEFVSRIDSPGGRTYYADSVKGRFTISRDNSKNTLYLQMNSLRA |
| | EDTAVYYCAKRRPTGVSGTFYDYWGQGTLVTVSSgggssgggsgGQ |
| | PREPQVYTLPPSRDELTKNQVSLTCLVKGFYPSDIAVEWESNGQP |
| | ENNYKTTPPVLDSDGSFFLYSKLTVDKSRWQQGNVFSCSVMHEA |
| | LHNHYTQKSLSLSPGggggsggsEVQLLESGGGLVQPGGSLRLSCAA |
| | SGFTFGTEQMWWVRQAPGKGLEFVSRIDSPGGRTYYADSVKGRF |
| | TISRDNSKNTLYLQMNSLRAEDTAVYYCAKRRPTGVSGTFYDY |
| | WGQGTLVTVSS |
| SEQ ID NO: 15 | EVQLLESGGGLVQPGGSLRLSCAASGFTFGTEQMWWVRQAPGK |
| | GLEFVSRIDSPGGRTYYADSVKGRFTISRDNSKNTLYLQMNSLRA |
| | EDTAVYYCAKRRPTGVSGTFYDYWGQGTLVTVSSggggsEVQLLE |
| | SGGGLVQPGGSLRLSCAASGFTFGTEQMWWVRQAPGKGLEFVS |
| | RIDSPGGRTYYADSVKGRFTISRDNSKNTLYLQMNSLRAEDTAV |
| | YYCAKRRPTGVSGTFYDYWGQGTLVTVSSggggsEVQLLESGGGL |
| | VQPGGSLRLSCAASGFTFGTEQMWWVRQAPGKGLEFVSRIDSPG |
| | GRTYYADSVKGRFTISRDNSKNTLYLQMNSLRAEDTAVYYCAK |
| | RRPTGVSGTFYDYWGQGTLVTVSS |
| SEQ ID NO: 16 | EVQLLESGGGLVQPGGSLRLSCAASGFTFGTEQMWWVRQAPGK |
| | GLEFVSRIDSPGGRTYYADSVKGRFTISRDNSKNTLYLQMNSLRA |
| | EDTAVYYCAKRRPTGVSGTFYDYWGQGTLVTVSSggggsEVQLLE |
| | SGGGLVQPGGSLRLSCAASGFTFGTEQMWWVRQAPGKGLEFVS |
| | RIDSPGGRTYYADSVKGRFTISRDNSKNTLYLQMNSLRAEDTAV |
| | YYCAKRRPTGVSGTFYDYWGQGTLVTVSSggggsEVQLLESGGGL |
| | VQPGGSLRLSCAASGFTFGTEQMWWVRQAPGKGLEFVSRIDSPG |
| | GRTYYADSVKGRFTISRDNSKNTLYLQMNSLRAEDTAVYYCAK |
| | RRPTGVSGTFYDYWGQGTLVTVSSEVQLLESGGGLVQPGGSLRL |
| | SCAASGFTFGTEQMWWVRQAPGKGLEFVSRIDSPGGRTYYADSV |
| | KGRFTISRDNSKNTLYLQMNSLRAEDTAVYYCAKRRPTGVSGTF |
| | YDYWGQGTLVTVSSggggggggsEVQLLESGGGLVQPGGSLRLSC |
| | AASGFTFGTEQMWWVRQAPGKGLEFVSRIDSPGGRTYYADSVK |
| | GRFTISRDNSKNTLYLQMNSLRAEDTAVYYCAKRRPTGVSGTFY |
| | DYWGQGTLVTVSSggggsggggsEVQLLESGGGLVQPGGSLRLSCA |
| | ASGFTFGTEQMWWVRQAPGKGLEFVSRIDSPGGRTYYADSVKG |
| | RFTISRDNSKNTLYLQMNSLRAEDTAVYYCAKRRPTGVSGTFYD |
| | YWGQGTLVTVSS |
| SEQ ID NO: 17 | QVQLVQSGAEVKKPGSSVKVSCKASGYTFSSNVISWVRQAPGQG |
| TGFb-scFv VH- | LEWMGGVIPIVDIANYAQRFKGRVTITADESTSTTYMELSSLRSE |
| VL1 G4S dimer | DTAVYYCASTLGLVLDAMDYWGQGTLVTVSSGGGGSGGGGSG |
| | GGGSALETVLTQSPGTLSLSPGERATLSCRASQSLGSSYLAWYQQ |
| | KPGQAPRLLIYGASSRAPGIPDRFSGSGSGTDFTLTISRLEPEDFAV |
| | YYCQQYADSPITFGQGTRLEIKggggsQVQLVQSGAEVKKPGSSVK |
| | VSCKASGYTFSSNVISWVRQAPGQGLEWMGGVIPIVDIANYAQR |
| | FKGRVTITADESTSTTYMELSSLRSEDTAVYYCASTLGLVLDAMD |
| | YWGQGTLVTVSSGGGGSGGGGSGGGGSALETVLTQSPGTLSLSP |
| | GERATLSCRASQSLGSSYLAWYQQKPGQAPRLLIYGASSRAPGIP |
| | DRFSGSGSGTDFTLTISRLEPEDFAVYYCQQYADSPITFGQGTRLE |
| | IK |
| SEQ ID NO: 18 | QVQLVQSGAEVKKPGSSVKVSCKASGYTFSSNVISWVRQAPGQG |
| TGFb-scFv VH- | LEWMGGVIPIVDIANYAQRFKGRVTITADESTSTTYMELSSLRSE |
| VL1 2xG4S | DTAVYYCASTLGLVLDAMDYWGQGTLVTVSSGGGGSGGGGSG |
| dimer | GGGSALETVLTQSPGTLSLSPGERATLSCRASQSLGSSYLAWYQQ |
| | KPGQAPRLLIYGASSRAPGIPDRFSGSGSGTDFTLTISRLEPEDFAV |
| | YYCQQYADSPITFGQGTRLEIKggggsggggsQVQLVQSGAEVKKPG |
| | SSVKVSCKASGYTFSSNVISWVRQAPGQGLEWMGGVIPIVDIANY |
| | AQRFKGRVTITADESTSTTYMELSSLRSEDTAVYYCASTLGLVLD |
| | AMDYWGQGTLVTVSSGGGGSGGGGSGGGGSALETVLTQSPGTL |
| | SLSPGERATLSCRASQSLGSSYLAWYQQKPGQAPRLLIYGASSRA |
| | PGIPDRFSGSGSGTDFTLTISRLEPEDFAVYYCQQYADSPITFGQGT |
| | RLEIK |
| SEQ ID NO: 19 | QVQLVQSGAEVKKPGSSVKVSCKASGYTFSSNVISWVRQAPGQG |
| TGFb-scFv VH- | LEWMGGVIPIVDIANYAQRFKGRVTITADESTSTTYMELSSLRSE |
| VL1 minibody | DTAVYYCASTLGLVLDAMDYWGQGTLVTVSSGGGGSGGGGSG |
| +hinge | GGGSALETVLTQSPGTLSLSPGERATLSCRASQSLGSSYLAWYQQ |
| | KPGQAPRLLIYGASSRAPGIPDRFSGSGSGTDFTLTISRLEPEDFAV |
| | YYCQQYADSPITFGQGTRLEIKGGSEPKSSDKTHTCPPCgggssgggsg |
| | GQPREPQVYTLPPSRDELTKNQVSLTCLVKGFYPSDIAVEWESNG |
| | QPENNYKTTPPVLDSDGSFFLYSKLTVDKSRWQQGNVFSCSVMH |
| | EALHNHYTQKSLSLSPG |
| SEQ ID NO: 20 | QVQLVQSGAEVKKPGSSVKVSCKASGYTFSSNVISWVRQAPGQG |
| TGFb-scFv VH- | LEWMGGVIPIVDIANYAQRFKGRVTITADESTSTTYMELSSLRSE |
| VL1 minibody | DTAVYYCASTLGLVLDAMDYWGQGTLVTVSSGGGGSGGGGSG |
| +hinge dimer | GGGSALETVLTQSPGTLSLSPGERATLSCRASQSLGSSYLAWYQQ |
| | KPGQAPRLLIYGASSRAPGIPDRFSGSGSGTDFTLTISRLEPEDFAV |
| | YYCQQYADSPITFGQGTRLEIKGGSEPKSsDKTHTCPPCgggssgggsg |
| | GQPREPQVYTLPPSRDELTKNQVSLTCLVKGFYPSDIAVEWESNG |
| | QPENNYKTTPPVLDSDGSFFLYSKLTVDKSRWQQGNVFSCSVMH |
| | EALHNHYTQKSLSLSPGggggsggsQVQLVQSGAEVKKPGSSVKVS |
| | CKASGYTFSSNVISWVRQAPGQGLEWMGGVIPIVDIANYAQRFK |
| | GRVTITADESTSTTYMELSSLRSEDTAVYYCASTLGLVLDAMDY |
| | WGQGTLVTVSSGGGGSGGGGSGGGGSALETVLTQSPGTLSLSPG |
| | ERATLSCRASQSLGSSYLAWYQQKPGQAPRLLIYGASSRAPGIPD |
| | RFSGSGSGTDFTLTISRLEPEDFAVYYCQQYADSPITFGQGTRLEI |
| | K |
| SEQ ID NO: 21 | QVQLVQSGAEVKKPGSSVKVSCKASGYTFSSNVISWVRQAPGQG |
| TGFb-scFv VH- | LEWMGGVIPIVDIANYAQRFKGRVTITADESTSTTYMELSSLRSE |
| VL1 minibody | DTAVYYCASTLGLVLDAMDYWGQGTLVTVSSGGGGSGGGGSG |
| | GGGSALETVLTQSPGTLSLSPGERATLSCRASQSLGSSYLAWYQQ |
| | KPGQAPRLLIYGASSRAPGIPDRFSGSGSGTDFTLTISRLEPEDFAV |
| | YYCQQYADSPITFGQGTRLEIKgggssgggsgGQPREPQVYTLPPSRD |
| | ELTKNQVSLTCLVKGFYPSDIAVEWESNGQPENNYKTTPPVLDSD |
| | GSFFLYSKLTVDKSRWQQGNVFSCSVMHEALHNHYTQKSLSLSP |
| | G |
| SEQ ID NO: 22 | QVQLVQSGAEVKKPGSSVKVSCKASGYTFSSNVISWVRQAPGQG |
| | LEWMGGVIPIVDIANYAQRFKGRVTITADESTSTTYMELSSLRSE |
| | DTAVYYCASTLGLVLDAMDYWGQGTLVTVSSGGGGSGGGGSG |
| | GGGSALETVLTQSPGTLSLSPGERATLSCRASQSLGSSYLAWYQQ |
| | KPGQAPRLLIYGASSRAPGIPDRFSGSGSGTDFTLTISRLEPEDFAV |
| | YYCQQYADSPITFGQGTRLEIKgggssgggsgGQPREPQVYTLPPSRD |
| | ELTKNQVSLTCLVKGFYPSDIAVEWESNGQPENNYKTTPPVLDSD |
| | GSFFLYSKLTVDKSRWQQGNVFSCSVMHEALHNHYTQKSLSLSP |
| | GggggsggsQVQLVQSGAEVKKPGSSVKVSCKASGYTFSSNVISWV |
| | RQAPGQGLEWMGGVIPIVDIANYAQRFKGRVTITADESTSTTYM |
| | ELSSLRSEDTAVYYCASTLGLVLDAMDYWGQGTLVTVSSGGGG |
| | SGGGGSGGGGSALETVLTQSPGTLSLSPGERATLSCRASQSLGSS |
| | YLAWYQQKPGQAPRLLIYGASSRAPGIPDRFSGSGSGTDFTLTISR |
| | LEPEDFAVYYCQQYADSPITFGQGTRLEIK |
| SEQ ID NO: 23 | QLQVQESGPGLVKPSETLSLTCTVSGGSISNSYFSWGWIRQPPGK |
| | GLEWIGSFYYGEKTYYNPSLKSRATISIDTSKSQFSLKLSSVTAAD |
| | TAVYYCPRGPTMIRGVIDSWGQGTLVTVSSGGGGSGGGGSGGG |
| | GSEIVLTQSPATLSLSPGERATLSCRASQSVRSYLAWYQQKPGQA |
| | PRLLIYDASNRATGIPARFSGSGSGTDFTLTISSLEPEDFAVYYCQQ |
| | RSNWPPTFGQGTKVEIKggggsQLQVQESGPGLVKPSETLSLTCTVS |
| | GGSISNSYFSWGWIRQPPGKGLEWIGSFYYGEKTYYNPSLKSRAT |
| | ISIDTSKSQFSLKLSSVTAADTAVYYCPRGPTMIRGVIDSWGQGTL |
| | VTVSSGGGGSGGGGSGGGGSEIVLTQSPATLSLSPGERATLSCRA |
| | SQSVRSYLAWYQQKPGQAPRLLIYDASNRATGIPARFSGSGSGTD |
| | FTLTISSLEPEDFAVYYCQQRSNWPPTFGQGTKVEIK |
| SEQ ID NO: 24 | QLQVQESGPGLVKPSETLSLTCTVSGGSISNSYFSWGWIRQPPGK |
| | GLEWIGSFYYGEKTYYNPSLKSRATISIDTSKSQFSLKLSSVTAAD |
| | TAVYYCPRGPTMIRGVIDSWGQGTLVTVSSGGGGSGGGGSGGG |
| | GSEIVLTQSPATLSLSPGERATLSCRASQSVRSYLAWYQQKPGQA |
| | PRLLIYDASNRATGIPARFSGSGSGTDFTLTISSLEPEDFAVYYCQQ |
| | RSNWPPTFGQGTKVEIKggggsggggsQLQVQESGPGLVKPSETLSLT |
| | CTVSGGSISNSYFSWGWIRQPPGKGLEWIGSFYYGEKTYYNPSLK |
| | SRATISIDTSKSQFSLKLSSVTAADTAVYYCPRGPTMIRGVIDSWG |
| | QGTLVTVSSGGGGSGGGGSGGGGSEIVLTQSPATLSLSPGERATL |
| | SCRASQSVRSYLAWYQQKPGQAPRLLIYDASNRATGIPARFSGSG |
| | SGTDFTLTISSLEPEDFAVYYCQQRSNWPPTFGQGTKVEIK |
| SEQ ID NO: 25 | QLQVQESGPGLVKPSETLSLTCTVSGGSISNSYFSWGWIRQPPGK |
| | GLEWIGSFYYGEKTYYNPSLKSRATISIDTSKSQFSLKLSSVTAAD |
| | TAVYYCPRGPTMIRGVIDSWGQGTLVTVSSGGGGSGGGGSGGG |
| | GSEIVLTQSPATLSLSPGERATLSCRASQSVRSYLAWYQQKPGQA |
| | PRLLIYDASNRATGIPARFSGSGSGTDFTLTISSLEPEDFAVYYCQQ |
| | RSNWPPTFGQGTKVEIKGGSEPKSSDKTHTCPPCgggssgggsgGQPR |
| | EPQVYTLPPSRDELTKNQVSLTCLVKGFYPSDIAVEWESNGQPEN |
| | NYKTTPPVLDSDGSFFLYSKLTVDKSRWQQGNVFSCSVMHEALH |
| | NHYTQKSLSLSPG |
| SEQ ID NO: 26 | QLQVQESGPGLVKPSETLSLTCTVSGGSISNSYFSWGWIRQPPGK |
| | GLEWIGSFYYGEKTYYNPSLKSRATISIDTSKSQFSLKLSSVTAAD |
| | TAVYYCPRGPTMIRGVIDSWGQGTLVTVSSGGGGSGGGGSGGG |
| | GSEIVLTQSPATLSLSPGERATLSCRASQSVRSYLAWYQQKPGQA |
| | PRLLIYDASNRATGIPARFSGSGSGTDFTLTISSLEPEDFAVYYCQQ |
| | RSNWPPTFGQGTKVEIKGGSEPKSSDKTHTCPPCgggssgggsgGQPR |
| | EPQVYTLPPSRDELTKNQVSLTCLVKGFYPSDIAVEWESNGQPEN |
| | NYKTTPPVLDSDGSFFLYSKLTVDKSRWQQGNVFSCSVMHEALH |
| | NHYTQKSLSLSPGggggsggsQLQVQESGPGLVKPSETLSLTCTVSG |
| | GSISNSYFSWGWIRQPPGKGLEWIGSFYYGEKTYYNPSLKSRATIS |
| | IDTSKSQFSLKLSSVTAADTAVYYCPRGPTMIRGVIDSWGQGTLV |
| | TVSSGGGGSGGGGSGGGGSEIVLTQSPATLSLSPGERATLSCRAS |
| | QSVRSYLAWYQQKPGQAPRLLIYDASNRATGIPARFSGSGSGTDF |
| | TLTISSLEPEDFAVYYCQQRSNWPPTFGQGTKVEIK |
| SEQ ID NO: 27 | QLQVQESGPGLVKPSETLSLTCTVSGGSISNSYFSWGWIRQPPGK |
| | GLEWIGSFYYGEKTYYNPSLKSRATISIDTSKSQFSLKLSSVTAAD |
| | TAVYYCPRGPTMIRGVIDSWGQGTLVTVSSGGGGSGGGGSGGG |
| | GSEIVLTQSPATLSLSPGERATLSCRASQSVRSYLAWYQQKPGQA |
| | PRLLIYDASNRATGIPARFSGSGSGTDFTLTISSLEPEDFAVYYCQQ |
| | RSNWPPTFGQGTKVEIKgggssgggsgGQPREPQVYTLPPSRDELTKN |
| | QVSLTCLVKGFYPSDIAVEWESNGQPENNYKTTPPVLDSDGSFFL |
| | YSKLTVDKSRWQQGNVFSCSVMHEALHNHYTQKSLSLSPG |
| SEQ ID NO: 28 | QLQVQESGPGLVKPSETLSLTCTVSGGSISNSYFSWGWIRQPPGK |
| | GLEWIGSFYYGEKTYYNPSLKSRATISIDTSKSQFSLKLSSVTAAD |
| | TAVYYCPRGPTMIRGVIDSWGQGTLVTVSSGGGGSGGGGSGGG |
| | GSEIVLTQSPATLSLSPGERATLSCRASQSVRSYLAWYQQKPGQA |
| | PRLLIYDASNRATGIPARFSGSGSGTDFTLTISSLEPEDFAVYYCQQ |
| | RSNWPPTFGQGTKVEIKgggssgggsgGQPREPQVYTLPPSRDELTKN |
| | QVSLTCLVKGFYPSDIAVEWESNGQPENNYKTTPPVLDSDGSFFL |
| | YSKLTVDKSRWQQGNVFSCSVMHEALHNHYTQKSLSLSPGggggs |
| | ggsQLQVQESGPGLVKPSETLSLTCTVSGGSISNSYFSWGWIRQPP |
| | GKGLEWIGSFYYGEKTYYNPSLKSRATISIDTSKSQFSLKLSSVTA |
| | ADTAVYYCPRGPTMIRGVIDSWGQGTLVTVSSGGGGSGGGGSG |
| | GGGSEIVLTQSPATLSLSPGERATLSCRASQSVRSYLAWYQQKPG |
| | QAPRLLIYDASNRATGIPARFSGSGSGTDFTLTISSLEPEDFAVYYC |
| | QQRSNWPPTFGQGTKVEIK |
| SEQ ID NO: 29 | EVQLLESGGGLVQPGGSLRLSCAASGFTFGQESMYWVRQAPGK |
| TGFbR2 VH2 | GLEWVSAISGSGGSTYYADSVKGRFTISRDNSKNTLYLQMNSLR |
| | AEDTAVYYCAKSGTRIKQGFDYWGQGTLVTVSS |
| SEQ ID NO: 30 | EVQLLESGGGLVQPGGSLRLSCAASGSTFTEYRMWWVRQAPGK |
| TGFbR2 hVH2 | GLEWVSAIEPIGHRTYYANSVRGRFTISRDNSKNTLYLQMNSLRA |
| | EDTAVYYCAKQAPGEKWARRWDLDYWGQGTLVTVSS |
| SEQ ID NO: 31 | EVQLLESGGGLVQPGGSLRLSCAASGFTFGTDQMWWVRQAPGK |
| TGFbR2 VH3 | GLEFVSRIDSPGGRTYYANSVKGRFTISRDNSKNTLYLQMNSLRA |
| | EDTAVYYCAKRQPAGVSGKYVDYWGQGTLVTVSS |
| SEQ ID NO: 32 | IPPHVPKSDVEMEAQKDASIHLSCNRTIHPLKHFNSDVMASDNGG |
| mTGFbR2ECD | AVKLPQLCKFCDVRLSTCDNQKSCMSNCSITAICEKPHEVCVAV |
| (I24-K31, F57- | WRKNDKNITLETVCHDPKLTYHGFTLEDAASPKCVMKEKKRAG |
| L185) | ETFFMCACNMEECNDYIIFSEEYTTSSPDL |
| SEQ ID NO: 33 | LQCFCHLCTKDNFTCVTDGLCFVSVTETTDKVIHNSMCIAEIDLIP |
| huTGFbR1 + 2 | RDRPFVCAPSSKTGSVTTTYCCNQDHCNKIELPTTVKSSPGLGPV |
| ECD v1 | EGGGGSTIPPHVQKSVNNDMIVTDNNGAVKFPQLCKFCDVRFST |
| | CDNQKSCMSNCSITSICEKPQEVCVAVWRKNDENITLETVCHDPK |
| | LPYHDFILEDAASPKCIMKEKKKPGETFFMCSCSSDECNDNIIFSE |
| | EYNTSNPD |
| | [hTGFbR1-ECD(L34-E125)-hTGFbR2-ECD(A44-D151)] |
| SEQ ID NO: 34 | QCFCHLCTKDNFTCVTDGLCFVSVTETTDKVIHNSMCIAEIDLIPR |
| huTGFbR1 + 2 | DRPFVCAPSSKTGSVTTTYCCNQDHCNGGGGSAVKFPQLCKFCD |
| ECD v2 | VRFSTCDNQKSCMSNCSITSICEKPQEVCVAVWRKNDENITLETV |
| | CHDPKLPYHDFILEDAASPKCIMKEKKKPGETFFMCSCSSDECND |
| | NIIFSE |
| | [hTGFbR1-ECD(Q35-N107)-4GS-4GS-hTGFbR2-ECD(L34-E125)] |
| SEQ ID NO: 35 | LQCFCHLCTKDNFTCETDGLCFVSVTETTDKVIHNSMCIAEIDLIP |
| mTGFbR1 + 2 | RDRPFVCAPSSKTGAVTTTYCCNQDHCNKIELPTTGPFSEKQSAG |
| ECD v1 | LGPVELGGGGSIPPHVPKSVNSDVMASDNGGAVKLPQLCKFCDV |
| | RLSTCDNQKSCMSNCSITAICEKPHEVCVAVWRKNDKNITLETVC |
| | HDPKLTYHGFTLEDAASPKCVMKEKKRAGETFFMCACNMEECN |
| | DYIIFSEEYTTSSPD |
| | [mTGFbR1-ECD(L30-L126)-4GS-mTGFbR2-ECD(I24-K31, F57- |
| | D184)] |
| SEQ ID NO: 36 | QCFCHLCTKDNFTCETDGLCFVSVTETTDKVIHNSMCIAEIDLIPR |
| huTGFbR1 + 2 | DRPFVCAPSSKTGAVTTTYCCNQDHCNGGGGSAVKLPQLCKFCD |
| ECD v2 | VRLSTCDNQKSCMSNCSITAICEKPHEVCVAVWRKNDKNITLET |
| | VCHDPKLTYHGFTLEDAASPKCVMKE |
| | [mTGFbR1-ECD(Q31-N107)-4GS-mTGFbR2-ECD(I24-K31, F57- |
| | E150)] |
| SEQ ID NO: 37 | METDTLLLWVLLLWVPGSTGIPPHVPKSDVEMEAQKDASIHLSC |
| Mouse TGFbR2 | NRTIHPLKHFNSDVMASDNGGAVKLPQLCKFCDVRLSTCDNQKS |
| ECD with IgKss | CMSNCSITAICEKPHEVCVAVWRKNDKNITLETVCHDPKLTYHG |
| | FTLEDAASPKCVMKEKKRAGETFFMCACNMEECNDYIIFSEEYTT |
| | SSPDL |
| SEQ ID NO: 38 | METDTLLLWVLLLWVPGSTGIPPHVPKSDVEMEAQKDASIHLSC |
| Mouse TGFbR2 | NRTIHPLKHFNSDVMASDNGGAVKLPQLCKFCDVRLSTCDNQKS |
| ECD dimer with | CMSNCSITAICEKPHEVCVAVWRKNDKNITLETVCHDPKLTYHG |
| 8(G4S) linker and | FTLEDAASPKCVMKEKKRAGETFFMCACNMEECNDYIIFSEEYTT |
| IgKss with flag | SSPDLGGGGSGGGGSGGGGSGGGGSGGGGSGGGGSGGGGSGGG |
| tag | GSIPPHVPKSDVEMEAQKDASIHLSCNRTIHPLKHFNSDVMASDN |
| | GGAVKLPQLCKFCDVRLSTCDNQKSCMSNCSITAICEKPHEVCV |
| | AVWRKNDKNITLETVCHDPKLTYHGFTLEDAASPKCVMKEKKR |
| | AGETFFMCACNMEECNDYIIFSEEYTTSSPDLGGGGS |
| | DYKDDDDK |
| SEQ ID NO: 39 | METDTLLLWVLLLWVPGSTGIPPHVPKSDVEMEAQKDASIHLSC |
| TGFbR2 ECD | NRTIHPLKHFNSDVMASDNGGAVKLPQLCKFCDVRLSTCDNQKS |
| dimer | CMSNCSITAICEKPHEVCVAVWRKNDKNITLETVCHDPKLTYHG |
| | FTLEDAASPKCVMKEKKRAGETFFMCACNMEECNDYIIFSEEYTT |
| | SSPDLGGGGSGGGGSGGGGSGGGGSGGGGSGGGGSGGGGSGGG |
| | GSIPPHVPKSDVEMEAQKDASIHLSCNRTIHPLKHFNSDVMASDN |
| | GGAVKLPQLCKFCDVRLSTCDNQKSCMSNCSITAICEKPHEVCV |
| | AVWRKNDKNITLETVCHDPKLTYHGFTLEDAASPKCVMKEKKR |
| | AGETFFMCACNMEECNDYIIFSEEYTTSSPDL |
| SEQ ID NO: 40 | LQCFCHLCTKDNFTCVTDGLCFVSVTETTDKVIHNSMCIAEIDLIP |
| huTGFbR2 ECD | RDRPFVCAPSSKTGSVTTTYCCNQDHCNKIELPTTVKSSPGLGPV |
| monomer1 | E |
| SEQ ID NO: 41 | IEVMYPPPYLDNEKSNGTIIHVKGKHLCPSPLFPGPSKP |
| CD28 hinge | |
| domain | |
| SEQ ID NO: 42 | FWVLVVVGGVLACYSLLVTVAFIIFWV |
| CD28 | |
| Transmembrane | |
| domain | |
| SEQ ID NO: 43 | IEVMYPPPYLDNEKSNGTIIHVKGKHLCPSPLFPGPSKPFWVLVVV |
| CD28 | GGVLACYSLLVTVAFIIFWV |
| hinge/ | |
| transmembrane | |
| domain | |
| SEQ ID NO: 44 | RSKRSRLLHSDYMNMTPRRPGPTRKHYQPYAPPRDFAAYRS |
| CD28 signal | |
| domain | |
| SEQ ID NO: 45 | RVKFSRSADAPAYQQGQNQLYNELNLGRREEYDVLDKRRGRDP |
| CD3z-1XX | EMGGKPRRKNPQEGLFNELQKDKMAEAFSEIGMKGERRRGKGH |
| mutant signaling | DGLFQGLSTATKDTFDALHMQALPPR |
| domain | |
| SEQ ID NO: 46 | IEVMYPPPYLDNEKSNGTIIHVKGKHLCPSPLFPGPSKPFWVLVVV |
| COMBINED | GGVLACYSLLVTVAFIIFWVRSKRSRLLHSDYMNMTPRRPGPTR |
| CAR | KHYQPYAPPRDFAAYRSRVKFSRSADAPAYQQGQNQLYNELNL |
| transmembrane | GRREEYDVLDKRRGRDPEMGGKPRRKNPQEGLFNELQKDKMAE |
| and intracellular | AFSEIGMKGERRRGKGHDGLFQGLSTATKDTFDALHMQALPPR |
| domains | |
| SEQ ID NO: 47 | MGWSCIILFLVATATGVHSEVQLQESGPGLVKPSETLSLTCTVSG |
| V1-01 anti-GCC | ASISHYYWSWFRQPAGKGLEWIGRIYPSGSTSYNPSLKSRVAMSV |
| CAR | DTPKNQFSLKLSSVTAADTAVYYCARDRSTGWSEWNSDLWGRG |
| | TLVTVSSIEVMYPPPYLDNEKSNGTIIHVKGKHLCPSPLFPGPSKPF |
| | WVLVVVGGVLACYSLLVTVAFIIFWVRSKRSRLLHSDYMNMTPR |
| | RPGPTRKHYQPYAPPRDFAAYRSRVKFSRSADAPAYQQGQNQLY |
| | NELNLGRREEYDVLDKRRGRDPEMGGKPRRKNPQEGLFNELQK |
| | DKMAEAFSEIGMKGERRRGKGHDGLFQGLSTATKDTFDALHMQ |
| | ALPPR |
| SEQ ID NO: 48 | MELGLSWVFLVAILEGVQCEVQLVESGGGLVQPGGSLRLSCAAS |
| V51 anti-GCC | GFTFSRYWMTWVRQAPGKGLEWVAKIRHDGGEKYYADSVKGR |
| CAR | FTISRDNAKNSLYLQMNSLRAEDTAVYYCTRDYNKDYWGQGTL |
| | VTVSSIEVMYPPPYLDNEKSNGTIIHVKGKHLCPSPLFPGPSKPFW |
| | VLVVVGGVLACYSLLVTVAFIIFWV |
| | RSKRSRLLHSDYMNMTPRRPGPTRKHYQPYAPPRDFAAYRSRVK |
| | FSRSADAPAYQQGQNQLYNELNLGRREEYDVLDKRRGRDPEMG |
| | GKPRRKNPQEGLFNELQKDKMAEAFSEIGMKGERRRGKGHDGL |
| | FQGLSTATKDTFDALHMQALPPR |
| SEQ ID NO: 49 | MGTSLLCWMALCLLGADHADAQVQLQQSGPELVKPGASVKISC |
| Anti-CD19 scFv | KASGYAFSSSWMNWVKQRPG |
| | KGLEWIGRIYPGDEDTNYSGKFKDKATLTADKSSTTAYMQLSSL |
| | TSEDSAVYFCARSLLYGDY |
| | LDYWGQGTTLTVSSGGGGSGGGGSGGGGSQIVLTQSPAIMSASP |
| | GEKVTMTCSASSSVSYMHW |
| | YQQKSGTSPKRWIYDTSKLASGVPDRFSGSGSGTSYFLTINNMEA |
| | EDAATYYCQQWNINPLTF |
| | GAGTKLELKRSDPTTTPAPRPPTPAPTIASQPLSLRPEACRPAAGG |
| | AVHTRGLDFACDIFWVLWVGGVLACYSLLVTVAFI |
| | IFWVRSKRSRLLHSDYMNMTPRRPGPTRKHYQPYAPPRDFAAYR |
| | SRDQRLPPDAHKPPGGGSFRTPIQEEQADAHSTLAKIRVKFSRSA |
| | DAPAYQQGQNQLYNELNL |
| | GRREEYDVLDKRRGRDPEMGGKPRRKNPQEGLYNELQKDKMA |
| | EAYSEIGMKGERRRGKGHDGL |
| | YQGLSTATKDTYDALHMQALPPR |
| V5 anti-GCC | MELGLSWVFLVAILEGVQCQVQLVESGGGLVQPGGSLRLSCTAS |
| CAR SEQ ID | GFTFSRYWMSWVRQAPGKGLEWVAKIRHDGGEKYYVDSVKGR |
| NO: 50 | FTISRDNAKNSLYLQMNSLRAEDTAVYYCATDYTRDVWGQGTA |
| | VTVSSRAAAIEVMYPPPYLDNEKSNGTIIHVKGKHLCPSPLFPGPS |
| | KPFWVLVVVGGVLACYSLLVTVAFIIFWV |
| | RSKRSRLLHSDYMNMTPRRPGPTRKHYQPYAPPRDFAAYRS |
| | RVKFSRSADAPAYQQGQNQLYNELNLGRREEYDVLDKRRGRDP |
| | EMGGKPRRKNPQEGLFNELQKDKMAEAFSEIGMKGERRRGKGH |
| | DGLFQGLSTATKDTFDALHMQALPPR (SEQ ID NO:) |
| V36 anti-GCC | MELGLSWVFLVAILEGVQCEVQLVESGGGLAQPGGSLRLSCAAS |
| CAR SEQ ID | GFTFSRYWMTWVRQAPGGRLEWVAKIKYDGSEKYYADSVKGR |
| NO: 51 | FTISRDNAKNSLYLQMDSLRAEDTAVYYCTRDYNKDYWGQGTL |
| | VTVSSRAAAIEVMYPPPYLDNEKSNGTIIHVKGKHLCPSPLFPGPS |
| | KPFWVLVVVGGVLACYSLLVTVAFIIFWV |
| | RSKRSRLLHSDYMNMTPRRPGPTRKHYQPYAPPRDFAAYRS |
| | RVKFSRSADAPAYQQGQNQLYNELNLGRREEYDVLDKRRGRDP |
| | EMGGKPRRKNPQEGLFNELQKDKMAEAFSEIGMKGERRRGKGH |
| | DGLFQGLSTATKDTFDALHMQALPPR (SEQ ID NO:) |
| V1 anti-GCC | MGWSCIILFLVATATGVHSQVQLQESGPGLVKPSETLSLTCTVSG |
| CAR | ASISHYYWSWFRQPAGKGLEWIGRIYPSGSTSYNPSLKSRVAMSV |
| SEQ ID NO: 52 | DTPKNQFSLNLSSVTAADTAVYYCARDRSTGWSEWNSDLWGRG |
| | TLVTVSSRAAAIEVMYPPPYLDNEKSNGTIIHVKGKHLCPSPLFPG |
| | PSKPFWVLVVVGGVLACYSLLVTVAFIIFWVRSKRSRLLHSDYM |
| | NMTPRRPGPTRKHYQPYAPPRDFAAYRSRVKFSRSADAPAYQQG |
| | QNQLYNELNLGRREEYDVLDKRRGRDPEMGGKPRRKNPQEGLF |
| | NELQKDKMAEAFSEIGMKGERRRGKGHDGLFQGLSTATKDTFD |
| | ALHMQALPPR (SEQ ID NO:) |
| V48 anti-GCC | MELGLSWVFLVAILEGVQCEVQLVESGGGLVQPGGSLRLTCAAS |
| CAR | GFTFSRYWMTWVRQAPGKGLEWVAKIRHDGGEKYYPDSVKGR |
| SEQ ID NO: 53 | FTVSRDNAKNSLYLQMDNLRAEDTAMYYCTRDYNKDLWGQGT |
| | LVTVSSRAAAIEVMYPPPYLDNEKSNGTIIHVKGKHLCPSPLFPGP |
| | SKPFWVLVVVGGVLACYSLLVTVAFIIFWV |
| | RSKRSRLLHSDYMNMTPRRPGPTRKHYQPYAPPRDFAAYRS |
| | RVKFSRSADAPAYQQGQNQLYNELNLGRREEYDVLDKRRGRDP |
| | EMGGKPRRKNPQEGLFNELQKDKMAEAFSEIGMKGERRRGKGH |
| | DGLFQGLSTATKDTFDALHMQALPPR (SEQ ID NO:) |
| V8 anti-GCC | QVQLVESGGGLVQPGGSLRLSCAASGFTFSRYWMSWVRQAPGK |
| CAR | GLEWVAKIKYDGSEKYYVDSVKGRFTISRDNAKNSVYLQMNSL |
| SEQ ID NO: 54 | RAEDTGVYYCATDFTRDVWGQGTTVTVSS |
| | RAAAIEVMYPPPYLDNEKSNGTIIHVKGKHLCPSPLFPGPSKPFW |
| | VLVVVGGVLACYSLLVTVAFIIFWV |
| | RSKRSRLLHSDYMNMTPRRPGPTRKHYQPYAPPRDFAAYRS |
| | RVKFSRSADAPAYQQGQNQLYNELNLGRREEYDVLDKRRGRDP |
| | EMGGKPRRKNPQEGLFNELQKDKMAEAFSEIGMKGERRRGKGH |
| | DGLFQGLSTATKDTFDALHMQALPPR ( |
| V9 anti-GCC | EVQLVESGGGLVQPGGSLRLSCAASGFTFSRYWMTWVRQAPGR |
| CAR | GLEWVAKIRYDGGEKYYVDSVKGRFTISRDNAKNSLYLQMNSL |
| SEQ ID NO: 55 | RAEDTAVYYCATDFTRDVWGQGTTVTVSS |
| | RAAAIEVMYPPPYLDNEKSNGTIIHVKGKHLCPSPLFPGPSKPFW |
| | VLVVVGGVLACYSLLVTVAFIIFWV |
| | RSKRSRLLHSDYMNMTPRRPGPTRKHYQPYAPPRDFAAYRS |
| | RVKFSRSADAPAYQQGQNQLYNELNLGRREEYDVLDKRRGRDP |
| | EMGGKPRRKNPQEGLFNELQKDKMAEAFSEIGMKGERRRGKGH |
| | DGLFQGLSTATKDTFDALHMQALPPR ( |
| V30 anti-GCC | QVQLVESGGGLVQPGGSLRLSCAASGFNFGRYWMSWVRQAPGK |
| CAR | GREWVAKIKYDGSEKYYVDSVKGRFTISRDNAKNSLYLQMNSL |
| SEQ ID NO: 56 | RAEDTAVYYCATDFTRDVWGQGTTVTVSS |
| | RAAAIEVMYPPPYLDNEKSNGTIIHVKGKHLCPSPLFPGPSKPFW |
| | VLVVVGGVLACYSLLVTVAFIIFWV |
| | RSKRSRLLHSDYMNMTPRRPGPTRKHYQPYAPPRDFAAYRS |
| | RVKFSRSADAPAYQQGQNQLYNELNLGRREEYDVLDKRRGRDP |
| | EMGGKPRRKNPQEGLFNELQKDKMAEAFSEIGMKGERRRGKGH |
| | DGLFQGLSTATKDTFDALHMQALPPR ( |
| V31 anti-GCC | QVQLVESGGGVVRPGGSLRLSCAASGFTFSRYWMSWVRQAPGK |
| CAR | GREWVAKIKYDGSEKYYADSVKGRFTISRDNAKNSLYLQMNSL |
| SEQ ID NO: 57 | RADDTAVYYCATDFTRDVWGQGTTVTVSS |
| | RAAAIEVMYPPPYLDNEKSNGTIIHVKGKHLCPSPLFPGPSKPFW |
| | VLVVVGGVLACYSLLVTVAFIIFWV |
| | RSKRSRLLHSDYMNMTPRRPGPTRKHYQPYAPPRDFAAYRS |
| | RVKFSRSADAPAYQQGQNQLYNELNLGRREEYDVLDKRRGRDP |
| | EMGGKPRRKNPQEGLFNELQKDKMAEAFSEIGMKGERRRGKGH |
| | DGLFQGLSTATKDTFDALHMQALPPR ( |
| SEQ ID NO: 58 | GSGATNFSLLKQAGDVEENPGP |
| P2A | |
| SEQ ID NO: 59 | GGGGS |
| G4S Linker | |
| SEQ ID NO: 60 | GGGGSGGGGS |
| 2x G4S Linker | |
| SEQ ID NO: 61 | GGGGSGGGGSGGGGS |
| Mouse CD19 | METDTLLLWVLLLWVPGSTGEVKLQQSGAELVRPGSSVKISCKA |
| CAR | SGYAFSSYWMNWVKQRPGQGLEWIGQIYPGDGDTNYNGKFKG |
| SEQ ID NO: 62 | QATLTADKSSSTAYMQLSGLTSEDSAVYFCARKTISSVVDFYFDY |
| | WGQGTTVTVSSGGGGSGGGGSGGGGSDIELTQSPKFMSTSVGDR |
| | VSVTCKASQNVGTNVAWYQQKPGQSPKPLIYSATYRNSGVPDRF |
| | TGSGSGTDFTLTITNVQSKDLADYFCQQYNRYPYTSGGGTKLEIK |
| | IEFMYPPPYLDNERSNGTIIHIKEKHLCHTQSSPKLFWALVVVAGV |
| | LFCYGLLVTVALCVIWTNSRRNRLLQSDYMNMTPRRPGLTRKPY |
| | QPYAPA |
| | RDFAAYRPRAKFSRSAETAANLQDPNQLYNELNLGRREEYDVLE |
| | KKRARDPEMGGKQQRRRNPQEGVYNALQKDKMAEAYSEIGTK |
| | GERRRGKGHDGLYQGLSTATKDTYDALHMQTLAPR |
| Human CD19 | MALPVTALLLPLALLLHAEVKLQQSGAELVRPGSSVKISCKASGY |
| CAR (with CD3- | AFSSYWMNWVKQRPGQGLEWIGQIYPGDGDTNYNGKFKGQAT |
| 1xx) | LTADKSSSTAYMQLSGLTSEDSAVYFCARKTISSVVDFYFDYWG |
| SEQ ID NO: 63 | QGTTVTVSSGGGGSGGGGSGGGGSDIELTQSPKFMSTSVGDRVS |
| | VTCKASQNVGTNVAWYQQKPGQSPKPLIYSATYRNSGVPDRFTG |
| | SGSGTDFTLTITNVQSKDLADYFCQQYNRYPYTSGGGTKLEIKRA |
| | AAIEVMYPPPYLDNEKSNGTIIHVKGKHLCPSPLFPGPSKPFWVLV |
| | VVGGVLACYSLLVTVAFIIFWVRSKRSRLLHSDYMNMTPRRPGP |
| | TRKHYQPYAPPRDFAAYRSRVKFSRSADAPAYQQGQNQLYNEL |
| | NLGRREEYDVLDKRRGRDPEMGGKPRRKNPQEGLFNELQKDKM |
| | AEAFSEIGMKGERRRGKGHDGLFQGLSTATKDTFDALHMQALPP |
| | R |

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above Description, but rather is as set forth in the following claims.

Having thus described several aspects of at least one embodiment of this invention, it is to be appreciated that various alterations, modifications, and improvements will readily be apparent to those skilled in the art. Such alterations, modifications, and improvements are intended to be part of this disclosure, and are intended to be within the spirit and scope of the invention. Accordingly, the foregoing description and drawings are by way of example only and the invention is described in detail by the claims that follow.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or the entire group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, (e.g., in Markush group or similar format) it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. The publications, websites and other reference materials referenced herein to describe the background of the invention and to provide additional detail regarding its practice are hereby incorporated by reference.

We claim:

1. A population of genetically engineered T cells, comprising a chimeric antigen receptor (CAR) that recognizes a cancer associated antigen and a TGFβ signaling pathway modulator.

2. The population of cells according to claim 1, wherein the CAR recognizes an antigen selected from the group consisting of ADGRE2, CLEC12, CAIX, CEA, CD5, CD7, CD10, CD19, CD20, CD22, CD30, CD33, CD34, CD38, CD41, CD44, CD49f, CD56, CD74, CD133, CD138, a cytomegalovirus (CMV) infected cell antigen, CEACAM 5, Claudin 18.2, EGP-2, EGP-40, EpCAM, erb-B2,3,4, FBP, Fetal acetylcholine receptor, folate receptor-a, GCC (also known as GUCY2C), GD2, GD3, HER-2, hTERT, IL-13R-a2, x-light chain, KDR, LeY, LI cell adhesion molecule, MAGE-AI, MUC1, MUC13, Mesothelin, NKG2D ligands, NY-ES0-1, oncofetal antigen (h5T4), PSCA, PSMA, PTK7, ROR1, TAG-72, TROP2, VEGF-R2, and WT-1.

3. The population of cells according to claim 1 or 2, wherein the TGFβ signaling pathway modulator binds TGFβ or a TGFβ receptor.

4. The population of cells according to any of the preceding claims, wherein the TGFβ signaling pathway modulator comprises an amino acid sequence selected from Table 1.

5. The population of cells according to any of preceding claims, wherein the CAR is a CD19 CAR or a GCC CAR.

6. The population of cells according to claim 1, wherein the cells are autologous.

7. The population of cells according to claim 1, wherein the cells are allogeneic.

8. The population of cells according to any one of the preceding claims, wherein the cells are genetically modified using a vector comprising a first nucleic acid encoding a CAR polypeptide and a second nucleic acid encoding a TGFβ signaling pathway modulator.

9. The population of cells according to any one of the preceding claims, wherein the cells are genetically modified using two vectors, first vector comprising a nucleic acid encoding a CAR polypeptide and a second vector comprising a nucleic acid encoding a TGFβ signaling pathway modulator.

10. The population of cells according to any one of the preceding claims, wherein the CAR comprises an intracellular signaling domain selected from the group consisting of CD3ζ-chain, CD97, 2B4 GDI la-CD18, CD2, ICOS, CD27, CD154, CDS, OX40, 4-1BB, DAP10, DAP12, CD28 signaling domain, or combinations and variations thereof.

11. The population of cells according to any one of the preceding claims, wherein the CAR comprises a transmembrane domain derived from a transmembrane domain selected from the group consisting of CD3, CD8, CD28, OX40, CD27, 4-1BB, DAP10, DAP12 or combinations thereof.

12. A vector comprising a first nucleic acid encoding a CAR polypeptide and a second nucleic acid encoding a TGFβ signaling pathway modulator.

13. The vector of claim 12, further comprising an internal ribosomal entry site.

14. A vector of claim 12, further comprising a 2A self-cleaving site.

15. An immune cell modified with a vector of any one of claims 12-14.

16. The immune cell of claim 15, wherein the cell is a T-cell.

17. A pharmaceutical composition comprising a population of immune cells according to claim 1.

18. A method of modulating an immune response in a host, the method comprising administering to the host a population of cells according to claim 1, wherein the modulation of immune response comprises one or more of the following by host immune cells: increase in IFNγ production; increase in IL-2 production; increase in antigen presentation; and increase in proliferation.

19. A method of treating or preventing cancer in a subject in need thereof, the method comprising administering to the subject an effective amount of a population of cells according to claim 1.

20. The method of claim 19, wherein the cancer is selected from the group consisting of leukemia, acute leukemia, acute lymphocytic leukemia, acute myelocytic leukemia, acute myeloblastic leukemia, acute promyelocyte leukemia, acute myelomonocytic leukemia, acute monocytic leukemia, acute erythroleukemia, chronic leukemia, chronic myelocytic leukemia, multiple myeloma, chronic lymphocytic leukemia, polycythemia vera, lymphoma, Hodgkin's lymphoma, non-Hodgkin's lymphoma, Waldenstrom's macroglobulinemia, heavy chain disease, solid tumors, sarcoma, carcinoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, hepatocellular carcinoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, colorectal carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, retinoblastoma, and metastasis thereof.


SEQUENCE LISTING (65 sequences)

SEQ ID NO: 1 - protein, 246 aa, Artificial Sequence
  source: /note=“Description of Artificial Sequence Synthetic polypeptide”
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSSGGGGSGGGGSGGGGSALETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKR

SEQ ID NO: 2 - protein, 244 aa, Artificial Sequence
  source: /note=“Description of Artificial Sequence Synthetic polypeptide”
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSSGGGGSGGGGSGGGGSETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKR

SEQ ID NO: 3 - protein, 244 aa, Artificial Sequence
  source: /note=“Description of Artificial Sequence Synthetic polypeptide”
ETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKRGGGGSGGGGSGGGGSQVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSS

SEQ ID NO: 4 - protein, 243 aa, Artificial Sequence
  source: /note=“Description of Artificial Sequence Synthetic polypeptide”
QLQVQESGPGLVKPSETLSLTCTVSGGSISNSYFSWGWIRQPPGKGLEWIGSFYYGEKTYYNPSLKSRATISIDTSKSQFSLKLSSVTAADTAVYYCPRGPTMIRGVIDSWGQGTLVTVSSGGGGSGGGGSGGGGSEIVLTQSPATLSLSPGERATLSCRASQSVRSYLAWYQQKPGQAPRLLIYDASNRATGIPARFSGSGSGTDFTLTISSLEPEDFAVYYCQQRSNWPPTFGQGTKVEIK

SEQ ID NO: 5 - protein, 243 aa, Artificial Sequence
  source: /note=“Description of Artificial Sequence Synthetic polypeptide”
EIVLTQSPATLSLSPGERATLSCRASQSVRSYLAWYQQKPGQAPRLLIYDASNRATGIPARFSGSGSGTDFTLTISSLEPEDFAVYYCQQRSNWPPTFGQGTKVEIKGGGGSGGGGSGGGGSQLQVQESGPGLVKPSETLSLTCTVSGGSISNSYFSWGWIRQPPGKGLEWIGSFYYGEKTYYNPSLKSRATISIDTSKSQFSLKLSSVTAADTAVYYCPRGPTMIRGVIDSWGQGTLVTVSS

SEQ ID NO: 6 - protein, 122 aa, Artificial Sequence
  source: /note=“Description of Artificial Sequence Synthetic polypeptide”
EVQLLESGGGLVQPGGSLRLSCAASGFTFTTYGMGWVRQAPGKGLEWVSWIEKTGNKTYYADSVKGRFTISRDNSKNTLYLQMNSLRAEDTAVYYCAKARHIKVRSRDFDYWGQGTLVTVSS

SEQ ID NO: 7 - protein, 123 aa, Artificial Sequence
  source: /note=“Description of Artificial Sequence Synthetic polypeptide”
EVQLLESGGGLVQPGGSLRLSCAASGFTFTTYGMGWVRQAPGKGLEWVSWIEKTGNKTYYADSVKGRFTISRDNSKNTLYLQMNSLRAEDTAVYYCAKAGRHIKVRSRDFDYWGQGTLVTVSS

SEQ ID NO: 8 - protein, 122 aa, Artificial Sequence
  source: /note=“Description of Artificial Sequence Synthetic polypeptide”
EVQLLESGGGLVQPGGSLRLSCAASGFTFGTEQMWWVRQAPGKGLEFVSRIDSPGGRTYYADSVKGRFTISRDNSKNTLYLQMNSLRAEDTAVYYCAKRRPTGVSGTFYDYWGQGTLVTVSS

SEQ ID NO: 9 - protein, 249 aa, Artificial Sequence
  source: /note=“Description of Artificial Sequence Synthetic polypeptide”
EVQLLESGGGLVQPGGSLRLSCAASGFTFGTEQMWWVRQAPGKGLEFVSRIDSPGGRTYYADSVKGRFTISRDNSKNTLYLQMNSLRAEDTAVYYCAKRRPTGVSGTFYDYWGQGTLVTVSSGGGGSEVQLLESGGGLVQPGGSLRLSCAASGFTFGTEQMWWVRQAPGKGLEFVSRIDSPGGRTYYADSVKGRFTISRDNSKNTLYLQMNSLRAEDTAVYYCAKRRPTGVSGTFYDYWGQGTLVTVSS

SEQ ID NO: 10 - protein, 254 aa, Artificial Sequence
  source: /note=“Description of Artificial Sequence Synthetic polypeptide”
EVQLLESGGGLVQPGGSLRLSCAASGFTFGTEQMWWVRQAPGKGLEFVSRIDSPGGRTYYADSVKGRFTISRDNSKNTLYLQMNSLRAEDTAVYYCAKRRPTGVSGTFYDYWGQGTLVTVSSGGGGSGGGGSEVQLLESGGGLVQPGGSLRLSCAASGFTFGTEQMWWVRQAPGKGLEFVSRIDSPGGRTYYADSVKGRFTISRDNSKNTLYLQMNSLRAEDTAVYYCAKRRPTGVSGTFYDYWGQGTLVTVSS

SEQ ID NO: 11 - protein, 255 aa, Artificial Sequence
  source: /note=“Description of Artificial Sequence Synthetic polypeptide”
EVQLLESGGGLVQPGGSLRLSCAASGFTFGTEQMWWVRQAPGKGLEFVSRIDSPGGRTYYADSVKGRFTISRDNSKNTLYLQMNSLRAEDTAVYYCAKRRPTGVSGTFYDYWGQGTLVTVSSGGSEPKSSDKTHTCPPCGGGSSGGGSGGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 385 aa, Artificial Sequence
  source: /note=“Description of Artificial Sequence Synthetic polypeptide”
EVQLLESGGGLVQPGGSLRLSCAASGFTFGTEQMWWVRQAPGKGLEFVSRIDSPGGRTYYADSVKGRFTISRDNSKNTLYLQMNSLRAEDTAVYYCAKRRPTGVSGTFYDYWGQGTLVTVSSGGSEPKSSDKTHTCPPCGGGSSGGGSGGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGSEVQLLESGGGLVQPGGSLRLSCAASGFTFGTEQMWWVRQAPGKGLEFVSRIDSPGGRTYYADSVKGRFTISRDNSKNTLYLQMNSLRAEDTAVYYCAKRRPTGVSGTFYDYWGQGTLVTVSS

SEQ ID NO: 13 - protein, 238 aa, Artificial Sequence
  source: /note=“Description of Artificial Sequence Synthetic polypeptide”
EVQLLESGGGLVQPGGSLRLSCAASGFTFGTEQMWWVRQAPGKGLEFVSRIDSPGGRTYYADSVKGRFTISRDNSKNTLYLQMNSLRAEDTAVYYCAKRRPTGVSGTFYDYWGQGTLVTVSSGGGSSGGGSGGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 368 aa, Artificial Sequence
  source: /note=“Description of Artificial Sequence Synthetic polypeptide”
EVQLLESGGGLVQPGGSLRLSCAASGFTFGTEQMWWVRQAPGKGLEFVSRIDSPGGRTYYADSVKGRFTISRDNSKNTLYLQMNSLRAEDTAVYYCAKRRPTGVSGTFYDYWGQGTLVTVSSGGGSSGGGSGGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGSEVQLLESGGGLVQPGGSLRLSCAASGFTFGTEQMWWVRQAPGKGLEFVSRIDSPGGRTYYADSVKGRFTISRDNSKNTLYLQMNSLRAEDTAVYYCAKRRPTGVSGTFYDYWGQGTLVTVSS

SEQ ID NO: 15 - protein, 376 aa, Artificial Sequence
  source: /note=“Description of Artificial Sequence Synthetic polypeptide”
EVQLLESGGGLVQPGGSLRLSCAASGFTFGTEQMWWVRQAPGKGLEFVSRIDSPGGRTYYADSVKGRFTISRDNSKNTLYLQMNSLRAEDTAVYYCAKRRPTGVSGTFYDYWGQGTLVTVSSGGGGSEVQLLESGGGLVQPGGSLRLSCAASGFTFGTEQMWWVRQAPGKGLEFVSRIDSPGGRTYYADSVKGRFTISRDNSKNTLYLQMNSLRAEDTAVYYCAKRRPTGVSGTFYDYWGQGTLVTVSSGGGGSEVQLLESGGGLVQPGGSLRLSCAASGFTFGTEQMWWVRQAPGKGLEFVSRIDSPGGRTYYADSVKGRFTISRDNSKNTLYLQMNSLRAEDTAVYYCAKRRPTGVSGTFYDYWGQGTLVTVSS

SEQ ID NO: 16 - protein, 762 aa, Artificial Sequence
  source: /note=“Description of Artificial Sequence Synthetic polypeptide”
EVQLLESGGGLVQPGGSLRLSCAASGFTFGTEQMWWVRQAPGKGLEFVSRIDSPGGRTYYADSVKGRFTISRDNSKNTLYLQMNSLRAEDTAVYYCAKRRPTGVSGTFYDYWGQGTLVTVSSGGGGSEVQLLESGGGLVQPGGSLRLSCAASGFTFGTEQMWWVRQAPGKGLEFVSRIDSPGGRTYYADSVKGRFTISRDNSKNTLYLQMNSLRAEDTAVYYCAKRRPTGVSGTFYDYWGQGTLVTVSSGGGGSEVQLLESGGGLVQPGGSLRLSCAASGFTFGTEQMWWVRQAPGKGLEFVSRIDSPGGRTYYADSVKGRFTISRDNSKNTLYLQMNSLRAEDTAVYYCAKRRPTGVSGTFYDYWGQGTLVTVSSEVQLLESGGGLVQPGGSLRLSCAASGFTFGTEQMWWVRQAPGKGLEFVSRIDSPGGRTYYADSVKGRFTISRDNSKNTLYLQMNSLRAEDTAVYYCAKRRPTGVSGTFYDYWGQGTLVTVSSGGGGSGGGGSEVQLLESGGGLVQPGGSLRLSCAASGFTFGTEQMWWVRQAPGKGLEFVSRIDSPGGRTYYADSVKGRFTISRDNSKNTLYLQMNSLRAEDTAVYYCAKRRPTGVSGTFYDYWGQGTLVTVSSGGGGSGGGGSEVQLLESGGGLVQPGGSLRLSCAASGFTFGTEQMWWVRQAPGKGLEFVSRIDSPGGRTYYADSVKGRFTISRDNSKNTLYLQMNSLRAEDTAVYYCAKRRPTGVSGTFYDYWGQGTLVTVSS

SEQ ID NO: 17 - protein, 495 aa, Artificial Sequence
  source: /note=“Description of Artificial Sequence Synthetic polypeptide”
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSSGGGGSGGGGSGGGGSALETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKGGGGSQVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSSGGGGSGGGGSGGGGSALETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIK

SEQ ID NO: 18 - protein, 500 aa, Artificial Sequence
  source: /note=“Description of Artificial Sequence Synthetic polypeptide”
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSSGGGGSGGGGSGGGGSALETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKGGGGSGGGGSQVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSSGGGGSGGGGSGGGGSALETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIK

SEQ ID NO: 19 - protein, 378 aa, Artificial Sequence
  source: /note=“Description of Artificial Sequence Synthetic polypeptide”
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSSGGGGSGGGGSGGGGSALETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKGGSEPKSSDKTHTCPPCGGGSSGGGSGGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 20 - protein, 631 aa, Artificial Sequence
  source: /note=“Description of Artificial Sequence Synthetic polypeptide”
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSSGGGGSGGGGSGGGGSALETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKGGSEPKSSDKTHTCPPCGGGSSGGGSGGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGSQVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSSGGGGSGGGGSGGGGSALETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIK

SEQ ID NO: 21 - protein, 361 aa, Artificial Sequence
  source: /note=“Description of Artificial Sequence Synthetic polypeptide”
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSSGGGGSGGGGSGGGGSALETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKGGGSSGGGSGGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 614 aa, Artificial Sequence
  source: /note=“Description of Artificial Sequence Synthetic polypeptide”
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSSGGGGSGGGGSGGGGSALETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKGGGSSGGGSGGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGSQVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSSGGGGSGGGGSGGGGSALETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIK

SEQ ID NO: 23 - protein, 491 aa, Artificial Sequence
  source: /note=“Description of Artificial Sequence Synthetic polypeptide”
QLQVQESGPGLVKPSETLSLTCTVSGGSISNSYFSWGWIRQPPGKGLEWIGSFYYGEKTYYNPSLKSRATISIDTSKSQFSLKLSSVTAADTAVYYCPRGPTMIRGVIDSWGQGTLVTVSSGGGGSGGGGSGGGGSEIVLTQSPATLSLSPGERATLSCRASQSVRSYLAWYQQKPGQAPRLLIYDASNRATGIPARFSGSGSGTDFTLTISSLEPEDFAVYYCQQRSNWPPTFGQGTKVEIKGGGGSQLQVQESGPGLVKPSETLSLTCTVSGGSISNSYFSWGWIRQPPGKGLEWIGSFYYGEKTYYNPSLKSRATISIDTSKSQFSLKLSSVTAADTAVYYCPRGPTMIRGVIDSWGQGTLVTVSSGGGGSGGGGSGGGGSEIVLTQSPATLSLSPGERATLSCRASQSVRSYLAWYQQKPGQAPRLLIYDASNRATGIPARFSGSGSGTDFTLTISSLEPEDFAVYYCQQRSNWPPTFGQGTKVEIK

SEQ ID NO: 24 - protein, 496 aa, Artificial Sequence
  source: /note=“Description of Artificial Sequence Synthetic polypeptide”
QLQVQESGPGLVKPSETLSLTCTVSGGSISNSYFSWGWIRQPPGKGLEWIGSFYYGEKTYYNPSLKSRATISIDTSKSQFSLKLSSVTAADTAVYYCPRGPTMIRGVIDSWGQGTLVTVSSGGGGSGGGGSGGGGSEIVLTQSPATLSLSPGERATLSCRASQSVRSYLAWYQQKPGQAPRLLIYDASNRATGIPARFSGSGSGTDFTLTISSLEPEDFAVYYCQQRSNWPPTFGQGTKVEIKGGGGSGGGGSQLQVQESGPGLVKPSETLSLTCTVSGGSISNSYFSWGWIRQPPGKGLEWIGSFYYGEKTYYNPSLKSRATISIDTSKSQFSLKLSSVTAADTAVYYCPRGPTMIRGVIDSWGQGTLVTVSSGGGGSGGGGSGGGGSEIVLTQSPATLSLSPGERATLSCRASQSVRSYLAWYQQKPGQAPRLLIYDASNRATGIPARFSGSGSGTDFTLTISSLEPEDFAVYYCQQRSNWPPTFGQGTKVEIK

SEQ ID NO: 25 - protein, 376 aa, Artificial Sequence
  source: /note=“Description of Artificial Sequence Synthetic polypeptide”
QLQVQESGPGLVKPSETLSLTCTVSGGSISNSYFSWGWIRQPPGKGLEWIGSFYYGEKTYYNPSLKSRATISIDTSKSQFSLKLSSVTAADTAVYYCPRGPTMIRGVIDSWGQGTLVTVSSGGGGSGGGGSGGGGSEIVLTQSPATLSLSPGERATLSCRASQSVRSYLAWYQQKPGQAPRLLIYDASNRATGIPARFSGSGSGTDFTLTISSLEPEDFAVYYCQQRSNWPPTFGQGTKVEIKGGSEPKSSDKTHTCPPCGGGSSGGGSGGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 26 - protein, 627 aa, Artificial Sequence
  source: /note=“Description of Artificial Sequence Synthetic polypeptide”
QLQVQESGPGLVKPSETLSLTCTVSGGSISNSYFSWGWIRQPPGKGLEWIGSFYYGEKTYYNPSLKSRATISIDTSKSQFSLKLSSVTAADTAVYYCPRGPTMIRGVIDSWGQGTLVTVSSGGGGSGGGGSGGGGSEIVLTQSPATLSLSPGERATLSCRASQSVRSYLAWYQQKPGQAPRLLIYDASNRATGIPARFSGSGSGTDFTLTISSLEPEDFAVYYCQQRSNWPPTFGQGTKVEIKGGSEPKSSDKTHTCPPCGGGSSGGGSGGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGSQLQVQESGPGLVKPSETLSLTCTVSGGSISNSYFSWGWIRQPPGKGLEWIGSFYYGEKTYYNPSLKSRATISIDTSKSQFSLKLSSVTAADTAVYYCPRGPTMIRGVIDSWGQGTLVTVSSGGGGSGGGGSGGGGSEIVLTQSPATLSLSPGERATLSCRASQSVRSYLAWYQQKPGQAPRLLIYDASNRATGIPARFSGSGSGTDFTLTISSLEPEDFAVYYCQQRSNWPPTFGQGTKVEIK

SEQ ID NO: 27 - protein, 359 aa, Artificial Sequence
  source: /note=“Description of Artificial Sequence Synthetic polypeptide”
QLQVQESGPGLVKPSETLSLTCTVSGGSISNSYFSWGWIRQPPGKGLEWIGSFYYGEKTYYNPSLKSRATISIDTSKSQFSLKLSSVTAADTAVYYCPRGPTMIRGVIDSWGQGTLVTVSSGGGGSGGGGSGGGGSEIVLTQSPATLSLSPGERATLSCRASQSVRSYLAWYQQKPGQAPRLLIYDASNRATGIPARFSGSGSGTDFTLTISSLEPEDFAVYYCQQRSNWPPTFGQGTKVEIKGGGSSGGGSGGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 28 - protein, 610 aa, Artificial Sequence
  source: /note=“Description of Artificial Sequence Synthetic polypeptide”
QLQVQESGPGLVKPSETLSLTCTVSGGSISNSYFSWGWIRQPPGKGLEWIGSFYYGEKTYYNPSLKSRATISIDTSKSQFSLKLSSVTAADTAVYYCPRGPTMIRGVIDSWGQGTLVTVSSGGGGSGGGGSGGGGSEIVLTQSPATLSLSPGERATLSCRASQSVRSYLAWYQQKPGQAPRLLIYDASNRATGIPARFSGSGSGTDFTLTISSLEPEDFAVYYCQQRSNWPPTFGQGTKVEIKGGGSSGGGSGGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGSQLQVQESGPGLVKPSETLSLTCTVSGGSISNSYFSWGWIRQPPGKGLEWIGSFYYGEKTYYNPSLKSRATISIDTSKSQFSLKLSSVTAADTAVYYCPRGPTMIRGVIDSWGQGTLVTVSSGGGGSGGGGSGGGGSEIVLTQSPATLSLSPGERATLSCRASQSVRSYLAWYQQKPGQAPRLLIYDASNRATGIPARFSGSGSGTDFTLTISSLEPEDFAVYYCQQRSNWPPTFGQGTKVEIK

SEQ ID NO: 29 - protein, 120 aa, Artificial Sequence
  source: /note=“Description of Artificial Sequence Synthetic polypeptide”
EVQLLESGGGLVQPGGSLRLSCAASGFTFGQESMYWVRQAPGKGLEWVSAISGSGGSTYYADSVKGRFTISRDNSKNTLYLQMNSLRAEDTAVYYCAKSGTRIKQGFDYWGQGTLVTVSS

SEQ ID NO: 30 - protein, 124 aa, Artificial Sequence
  source: /note=“Description of Artificial Sequence Synthetic polypeptide”
EVQLLESGGGLVQPGGSLRLSCAASGSTFTEYRMWWVRQAPGKGLEWVSAIEPIGHRTYYANSVRGRFTISRDNSKNTLYLQMNSLRAEDTAVYYCAKQAPGEKWARRWDLDYWGQGTLVTVSS

SEQ ID NO: 31 - protein, 122 aa, Artificial Sequence
  source: /note=“Description of Artificial Sequence Synthetic polypeptide”
EVQLLESGGGLVQPGGSLRLSCAASGFTFGTDQMWWVRQAPGKGLEFVSRIDSPGGRTYYANSVKGRFTISRDNSKNTLYLQMNSLRAEDTAVYYCAKRQPAGVSGKYVDYWGQGTLVTVSS

SEQ ID NO: 32 - protein, 162 aa, Mus sp.
IPPHVPKSDVEMEAQKDASIHLSCNRTIHPLKHFNSDVMASDNGGAVKLPQLCKFCDVRLSTCDNQKSCMSNCSITAICEKPHEVCVAVWRKNDKNITLETVCHDPKLTYHGFTLEDAASPKCVMKEKKRAGETFFMCACNMEECNDYIIFSEEYTTSSPDL

SEQ ID NO: 33 - protein, 234 aa, Artificial Sequence
  source: /note=“Description of Artificial Sequence Synthetic polypeptide”
LQCFCHLCTKDNFTCVTDGLCFVSVTETTDKVIHNSMCIAEIDLIPRDRPFVCAPSSKTGSVTTTYCCNQDHCNKIELPTTVKSSPGLGPVEGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 34 - protein, 186 aa, Artificial Sequence
  source: /note=“Description of Artificial Sequence Synthetic polypeptide”
QCFCHLCTKDNFTCVTDGLCFVSVTETTDKVIHNSMCIAEIDLIPRDRPFVCAPSSKTGSVTTTYCCNQDHCNGGGGSAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSE

SEQ ID NO: 35 - protein, 238 aa, Artificial Sequence
  source: /note=“Description of Artificial Sequence Synthetic polypeptide”
LQCFCHLCTKDNFTCETDGLCFVSVTETTDKVIHNSMCIAEIDLIPRDRPFVCAPSSKTGAVTTTYCCNQDHCNKIELPTTGPFSEKQSAGLGPVELGGGGSIPPHVPKSVNSDVMASDNGGAVKLPQLCKFCDVRLSTCDNQKSCMSNCSITAICEKPHEVCVAVWRKNDKNITLETVCHDPKLTYHGFTLEDAASPKCVMKEKKRAGETFFMCACNMEECNDYIIFSEEYTTSSPD

SEQ ID NO: 36 - protein, 160 aa, Artificial Sequence
  source: /note=“Description of Artificial Sequence Synthetic polypeptide”
QCFCHLCTKDNFTCETDGLCFVSVTETTDKVIHNSMCIAEIDLIPRDRPFVCAPSSKTGAVTTTYCCNQDHCNGGGGSAVKLPQLCKFCDVRLSTCDNQKSCMSNCSITAICEKPHEVCVAVWRKNDKNITLETVCHDPKLTYHGFTLEDAASPKCVMKE

SEQ ID NO: 37 - protein, 182 aa, Artificial Sequence
  source: /note=“Description of Artificial Sequence Synthetic polypeptide”
METDTLLLWVLLLWVPGSTGIPPHVPKSDVEMEAQKDASIHLSCNRTIHPLKHFNSDVMASDNGGAVKLPQLCKFCDVRLSTCDNQKSCMSNCSITAICEKPHEVCVAVWRKNDKNITLETVCHDPKLTYHGFTLEDAASPKCVMKEKKRAGETFFMCACNMEECNDYIIFSEEYTTSSPDL

SEQ ID NO: 38 - protein, 397 aa, Artificial Sequence
  source: /note=“Description of Artificial Sequence Synthetic polypeptide”
METDTLLLWVLLLWVPGSTGIPPHVPKSDVEMEAQKDASIHLSCNRTIHPLKHFNSDVMASDNGGAVKLPQLCKFCDVRLSTCDNQKSCMSNCSITAICEKPHEVCVAVWRKNDKNITLETVCHDPKLTYHGFTLEDAASPKCVMKEKKRAGETFFMCACNMEECNDYIIFSEEYTTSSPDLGGGGSGGGGSGGGGSGGGGSGGGGSGGGGSGGGGSGGGGSIPPHVPKSDVEMEAQKDASIHLSCNRTIHPLKHFNSDVMASDNGGAVKLPQLCKFCDVRLSTCDNQKSCMSNCSITAICEKPHEVCVAVWRKNDKNITLETVCHDPKLTYHGFTLEDAASPKCVMKEKKRAGETFFMCACNMEECNDYIIFSEEYTTSSPDLGGGGSDYKDDDDK

SEQ ID NO: 39 - protein, 384 aa, Artificial Sequence
  source: /note=“Description of Artificial Sequence Synthetic polypeptide”
METDTLLLWVLLLWVPGSTGIPPHVPKSDVEMEAQKDASIHLSCNRTIHPLKHFNSDVMASDNGGAVKLPQLCKFCDVRLSTCDNQKSCMSNCSITAICEKPHEVCVAVWRKNDKNITLETVCHDPKLTYHGFTLEDAASPKCVMKEKKRAGETFFMCACNMEECNDYIIFSEEYTTSSPDLGGGGSGGGGSGGGGSGGGGSGGGGSGGGGSGGGGSGGGGSIPPHVPKSDVEMEAQKDASIHLSCNRTIHPLKHFNSDVMASDNGGAVKLPQLCKFCDVRLSTCDNQKSCMSNCSITAICEKPHEVCVAVWRKNDKNITLETVCHDPKLTYHGFTLEDAASPKCVMKEKKRAGETFFMCACNMEECNDYIIFSEEYTTSSPDL

SEQ ID NO: 40 - protein, 92 aa, Homo sapiens
LQCFCHLCTKDNFTCVTDGLCFVSVTETTDKVIHNSMCIAEIDLIPRDRPFVCAPSSKTGSVTTTYCCNQDHCNKIELPTTVKSSPGLGPVE

SEQ ID NO: 41 - protein, 39 aa, Unknown
  source: /note=“Description of Unknown CD28 hinge domain sequence”
IEVMYPPPYLDNEKSNGTIIHVKGKHLCPSPLFPGPSKP

SEQ ID NO: 42 - protein, 27 aa, Unknown
  source: /note=“Description of Unknown CD28 transmembrane domain sequence”
FWVLVVVGGVLACYSLLVTVAFIIFWV

SEQ ID NO: 43 - protein, 66 aa, Artificial Sequence
  source: /note=“Description of Artificial Sequence Synthetic polypeptide”
IEVMYPPPYLDNEKSNGTIIHVKGKHLCPSPLFPGPSKPFWVLVVVGGVLACYSLLVTVAFIIFWV

SEQ ID NO: 44 - protein, 41 aa, Unknown
  source: /note=“Description of Unknown CD28 signal domain sequence”
RSKRSRLLHSDYMNMTPRRPGPTRKHYQPYAPPRDFAAYRS

SEQ ID NO: 45 - protein, 112 aa, Artificial Sequence
  source: /note=“Description of Artificial Sequence Synthetic polypeptide”
RVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 46 - protein, 219 aa, Artificial Sequence
  source: /note=“Description of Artificial Sequence Synthetic polypeptide”
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 47 - protein, 359 aa, Artificial Sequence
  source: /note=“Description of Artificial Sequence Synthetic polypeptide”
MGWSCIILFLVATATGVHSEVQLQESGPGLVKPSETLSLTCTVSGASISHYYWSWFRQPAGKGLEWIGRIYPSGSTSYNPSLKSRVAMSVDTPKNQFSLKLSSVTAADTAVYYCARDRSTGWSEWNSDLWGRGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 48 - protein, 353 aa, Artificial Sequence
  source: /note=“Description of Artificial Sequence Synthetic polypeptide”
MELGLSWVFLVAILEGVQCEVQLVESGGGLVQPGGSLRLSCAASGFTFSRYWMTWVRQAPGKGLEWVAKIRHDGGEKYYADSVKGRFTISRDNAKNSLYLQMNSLRAEDTAVYYCTRDYNKDYWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 49 - protein, 526 aa, Artificial Sequence
  source: /note=“Description of Artificial Sequence Synthetic polypeptide”
MGTSLLCWMALCLLGADHADAQVQLQQSGPELVKPGASVKISCKASGYAFSSSWMNWVKQRPGKGLEWIGRIYPGDEDTNYSGKFKDKATLTADKSSTTAYMQLSSLTSEDSAVYFCARSLLYGDYLDYWGQGTTLTVSSGGGGSGGGGSGGGGSQIVLTQSPAIMSASPGEKVTMTCSASSSVSYMHWYQQKSGTSPKRWIYDTSKLASGVPDRFSGSGSGTSYFLTINNMEAEDAATYYCQQWNINPLTFGAGTKLELKRSDPTTTPAPRPPTPAPTIASQPLSLRPEACRPAAGGAVHTRGLDFACDIFWVLWVGGVLACYSLLVTVAFIIFWVRSKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 357 aa, Artificial Sequence
  source: /note=“Description of Artificial Sequence Synthetic polypeptide”
MELGLSWVFLVAILEGVQCQVQLVESGGGLVQPGGSLRLSCTASGFTFSRYWMSWVRQAPGKGLEWVAKIRHDGGEKYYVDSVKGRFTISRDNAKNSLYLQMNSLRAEDTAVYYCATDYTRDVWGQGTAVTVSSRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 51 - protein, 357 aa, Artificial Sequence
  source: /note=“Description of Artificial Sequence Synthetic polypeptide”
MELGLSWVFLVAILEGVQCEVQLVESGGGLAQPGGSLRLSCAASGFTFSRYWMTWVRQAPGGRLEWVAKIKYDGSEKYYADSVKGRFTISRDNAKNSLYLQMDSLRAEDTAVYYCTRDYNKDYWGQGTLVTVSSRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 52 - protein, 363 aa, Artificial Sequence
  source: /note=“Description of Artificial Sequence Synthetic polypeptide”
MGWSCIILFLVATATGVHSQVQLQESGPGLVKPSETLSLTCTVSGASISHYYWSWFRQPAGKGLEWIGRIYPSGSTSYNPSLKSRVAMSVDTPKNQFSLNLSSVTAADTAVYYCARDRSTGWSEWNSDLWGRGTLVTVSSRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 53 - protein, 357 aa, Artificial Sequence
  source: /note=“Description of Artificial Sequence Synthetic polypeptide”
MELGLSWVFLVAILEGVQCEVQLVESGGGLVQPGGSLRLTCAASGFTFSRYWMTWVRQAPGKGLEWVAKIRHDGGEKYYPDSVKGRFTVSRDNAKNSLYLQMDNLRAEDTAMYYCTRDYNKDLWGQGTLVTVSSRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 54 - protein, 338 aa, Artificial Sequence
  source: /note=“Description of Artificial Sequence Synthetic polypeptide”
QVQLVESGGGLVQPGGSLRLSCAASGFTFSRYWMSWVRQAPGKGLEWVAKIKYDGSEKYYVDSVKGRFTISRDNAKNSVYLQMNSLRAEDTGVYYCATDFTRDVWGQGTTVTVSSRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 55 - protein, 338 aa, Artificial Sequence
  source: /note=“Description of Artificial Sequence Synthetic polypeptide”
EVQLVESGGGLVQPGGSLRLSCAASGFTFSRYWMTWVRQAPGRGLEWVAKIRYDGGEKYYVDSVKGRFTISRDNAKNSLYLQMNSLRAEDTAVYYCATDFTRDVWGQGTTVTVSSRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 56 - protein, 338 aa, Artificial Sequence
  source: /note=“Description of Artificial Sequence Synthetic polypeptide”
QVQLVESGGGLVQPGGSLRLSCAASGFNFGRYWMSWVRQAPGKGREWVAKIKYDGSEKYYVDSVKGRFTISRDNAKNSLYLQMNSLRAEDTAVYYCATDFTRDVWGQGTTVTVSSRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 57 - protein, 338 aa, Artificial Sequence
  source: /note=“Description of Artificial Sequence Synthetic polypeptide”
QVQLVESGGGVVRPGGSLRLSCAASGFTFSRYWMSWVRQAPGKGREWVAKIKYDGSEKYYADSVKGRFTISRDNAKNSLYLQMNSLRADDTAVYYCATDFTRDVWGQGTTVTVSSRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 58 - protein, 22 aa, Artificial Sequence
  source: /note=“Description of Artificial Sequence Synthetic peptide”
GSGATNFSLLKQAGDVEENPGP

SEQ ID NO: 59 - protein, 5 aa, Artificial Sequence
  source: /note=“Description of Artificial Sequence Synthetic peptide”
GGGGS

SEQ ID NO: 60 - protein, 10 aa, Artificial Sequence
  source: /note=“Description of Artificial Sequence Synthetic peptide”
GGGGSGGGGS

SEQ ID NO: 61 - protein, 15 aa, Artificial Sequence
  source: /note=“Description of Artificial Sequence Synthetic peptide”
GGGGSGGGGSGGGGS

SEQ ID NO: 62 - protein, 481 aa, Artificial Sequence
  source: /note=“Description of Artificial Sequence Synthetic polypeptide”
METDTLLLWVLLLWVPGSTGEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 63 - protein, 485 aa, Artificial Sequence
  source: /note=“Description of Artificial Sequence Synthetic polypeptide”
MALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 64 - protein, 50 aa, Artificial Sequence
  source: /note=“Description of Artificial Sequence Synthetic polypeptide”
  SITE (1)..(50): /note=“This sequence may encompass 1-10 ′Gly Gly Gly Gly Ser′ repeating units”
GGGGSGGGGSGGGGSGGGGSGGGGSGGGGSGGGGSGGGGSGGGGSGGGGS

SEQ ID NO: 65 - protein, 40 aa, Artificial Sequence
  source: /note=“Description of Artificial Sequence Synthetic polypeptide”
GGGGSGGGGSGGGGSGGGGSGGGGSGGGGSGGGGSGGGGS